(19) United States
(12) Patent Application Publication
(10) Pub. No.: US 2024/0307361 A1
(43) Pub. Date: Sep. 19, 2024

(54) METHOD FOR DIAGNOSING AND TREATING IRRITABLE BOWEL SYNDROME

(71) Applicant: Centre for Chinese Herbal Medicine Drug Development Limited, Hong Kong (CN)

(72) Inventors: Zhaoxiang BIAN, Hong Kong (CN); Hoi Leong Xavier WONG, Hong Kong (CN); Lixiang ZHAI, Hong Kong (CN); Johnson Yiu-Nam LAU, Hong Kong (CN)

(21) Appl. No.: 18/169,926

(22) Filed: Feb. 16, 2023

(51) Int. Cl. A61K 31/40; A61P 1/00; G01N 33/569; G01N 33/94

(52) U.S. Cl. CPC A61K 31/40; A61P 1/00; G01N 33/56911; G01N 33/94

(57) ABSTRACT

Provided herein is a method of diagnosing irritable bowel syndrome in a subject involving determining the amount of at least one of Ruminococcus gnavus, phenethylamine, tryptamine and tyramine in a fecal sample obtained from the subject. Also provided is a method of treating irritable bowel syndrome in a subject in need thereof involving the administration of a therapeutically effective amount of a trace amine-associated receptor 1 inhibitor.

### REFERENCE TO SEQUENCE LISTING

The Sequence Listing identified as Sequence_Listing_P24906US00.xml; Size: 5,077 bytes; and Date of Creation: Jan. 17, 2023, filed herewith, is hereby incorporated by reference in its entirety.

### TECHNICAL FIELD

The present disclosure relates to methods for diagnosing and/or treating irritable bowel syndrome.

### BACKGROUND

Irritable bowel syndrome (IBS) is the most prevalent functional bowel disorder and is characterized by an array of GI symptoms, including abdominal pain, bloating, abdominal distention, and bowel habit abnormalities. Despite the high prevalence of IBS, a lack of understanding of the pathogenetic mechanisms involved impedes research progress. Accordingly, treatment options for IBS are limited, and most therapeutic approaches focus on relieving symptoms only. Emerging evidence has revealed that the gut microbiota plays an important role in the pathophysiology of IBS, particularly in the diarrhea-predominant subtype (IBS-D). Transplantation of fecal microbiota from patients with IBS-D to recipient germ-free mice results in IBS-like symptoms, including accelerated GI transit and intestinal barrier dysfunction. Despite the strong association between gut dysbiosis and IBS pathogenesis, the exact pathogenic mechanisms involved in gut microbiota-mediated IBS development remain unclear.

Serotonin (5-HT), an important neurotransmitter derived from tryptophan, is released from enterochromaffin (EC) cells in the GI tract and has been shown to modulate gut motility and hypersensitivity functions. Several investigators have shown that increased production of peripheral 5-HT from intestinal EC cells contributes to GI symptoms in IBS-D, and that there is a strong association between peripheral 5-HT levels and IBS-D severity in humans. Therapeutic interventions targeting various 5-HT receptors have demonstrated moderate clinical efficacy in the management of IBS, but their therapeutic potential is hampered by adverse drug reactions, including ischemic colitis and severe constipation. Recent evidence suggests that the gut microbiota is involved in regulating peripheral 5-HT levels, whereas the identity of the bacterial species responsible for 5-HT dysregulation in IBS-D remains unclear, and the molecular mechanisms by which the gut microbiota modulates 5-HT production are unknown. Understanding the molecular axis involved may facilitate the identification of novel bacterial and molecular targets for the development of better therapeutic options for IBS-D.

Despite advances in understanding the pathogenesis of IBS, there still exists a need for improved methods of treating and/or diagnosing IBS.

### SUMMARY

The present disclosure relates to a pathogenesis of irritable bowel syndrome, which involves changes in GI motility and intestinal secretion, both of which are modulated by increased serotonin synthesis in the gut. As disclosed herein, the bacterium Ruminococcus gnavus is identified to play a key pathogenic role in IBS-D. Monocolonization of germ-free mice with R. gnavus induced IBS-D-like symptoms, including increased GI transit and colonic secretion, by stimulating the production of peripheral serotonin. From a metabolic perspective, the phenethylamine and tryptamine derived from R. gnavus-mediated catabolism of dietary phenylalanine and tryptophan have been identified as directly stimulating serotonin biosynthesis in intestinal enterochromaffin cells, which was mediated through the activation of a G-protein coupled receptor (TAAR1), thereby contributing to elevated GI transit and colonic secretion in IBS-D. Molecular and pathogenetic insights into how gut microbial metabolites derived from dietary essential amino acids affect serotonin-dependent control of gut motility are provided. It was found that the gut bacterium Ruminococcus gnavus was highly enriched in the fecal samples of the IBS-D patient cohort and showed that monoassociation of IBS-D-associated strain of R. gnavus induced IBS-D-like symptoms in germ-free mice by stimulating the production of peripheral 5-HT. Furthermore, it was discovered that R. gnavus facilitated the shunting of phenylalanine and tryptophan to phenethylamine and tryptamine, which, in turn, stimulated the biosynthesis of 5-HT in intestinal EC cells through the activation of trace amine-associated receptor 1 (TAAR1). Preclinical studies have shown that pharmacological antagonism of TAAR1 alleviates IBS-D-like symptoms in antibiotic-treated mice colonized with R. gnavus or transplanted with fecal microbiota from patients with IBS-D, revealing the therapeutic potential of targeting the phenethylamine/tryptamine-TAAR1 signaling axis for the management of IBS-D.

In a first aspect, provided herein is a method of treating irritable bowel syndrome (IBS) in a subject in need thereof, the method comprising: administering a therapeutically effective amount of a trace amine-associated receptor 1 (TAAR1) inhibitor to the subject.

In certain embodiments, the IBS is a diarrhea-predominant subtype (IBS-D).

In certain embodiments, the TAAR1 inhibitor selectively binds TAAR1.

In certain embodiments, the TAAR1 inhibitor is a small molecule.

In certain embodiments, the TAAR1 inhibitor is a compound of Formula 1:

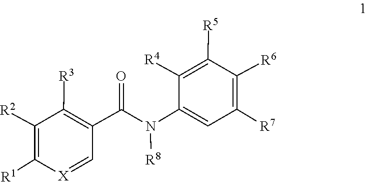

or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆alkyl, C₁-C₆alkoxy, C₁-C₆alkyl substituted by halogen, C₁-C₆ alkoxy substituted by halogen, cyano, NO₂, —(CH₂)ₚS(O)₂R, —OS(O)₂NR′R″, C₁-C₆alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen,
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆alkyl,

### X is —C(R⁹)═ or —N═;

R⁹ is hydrogen, C₁-C₆ alkoxy, NO₂ or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆alkyl, (CH₂)ₙ-phenyl optionally substituted by halogen or (CH₂)ₙ—O—C₁-C₆ alkyl.

In certain embodiments, the TAAR1 inhibitor is N-(3-ethoxy-phenyl)-4-pyrrolidin-1-yl-3-trifluoromethyl-benzamide.

In certain embodiments, the subject is a human, a non-human primate, a rodent, a canine, a feline, a bovine, or an equine.

In certain embodiments, the subject is a human.

In certain embodiments, a fecal sample obtained from the subject comprises a higher amount of one or more markers selected from the group consisting of Ruminococcus gnavus, phenethylamine, tryptamine and tyramine than an average amount of the one or more markers in fecal samples obtained from healthy controls.

In certain embodiments, the method further comprises the step of: providing a fecal sample obtained from the subject; determining the amount of one or more markers selected from the group consisting of phenethylamine, tryptamine and tyramine in the fecal sample; and determining based on the amount of the one or more markers in the fecal sample that the subject has IBS prior to the step of administering the TAAR1 inhibitor to the subject.

In certain embodiments, the one or more markers is phenethylamine and tyramine.

In certain embodiments, the method further comprises the step of: providing a fecal sample obtained from the subject; determining the amount of Ruminococcus gnavus in the fecal sample; and determining based on the amount of Ruminococcus gnavus in the fecal sample that the subject has IBS prior to the step of administering the TAAR1 inhibitor to the subject.

In a second aspect, provided herein is a method of treating diarrhea-predominant irritable bowel syndrome (IBS-D) in a subject in need thereof, the method comprising: providing a fecal sample obtained from the subject; determining the amount of one or more markers selected from the group consisting of Ruminococcus gnavus, phenethylamine, tryptamine and tyramine in the fecal sample; determining based on the amount of the one or more markers in the fecal sample that the subject has IBS; and administering a therapeutically effective amount of a trace amine-associated receptor 1 (TAAR1) inhibitor to the subject.

In certain embodiments, the TAAR1 inhibitor is a compound of Formula 1:

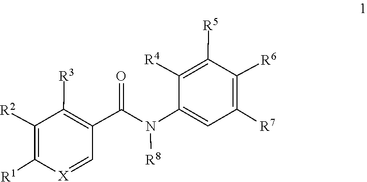

or a pharmaceutically acceptable salt thereof, wherein
n is 0, 1, 2 or 3;
p is 0 or 1,
R¹ is hydrogen, halogen. C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen,
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl:
R⁶ is hydrogen, (C₁-C₆ alkoxy, cyano, nitro, C₁-C₆ alkyl, phenyl, C₁-C₆ alkyl substituted by halogen, C₁-C₆ alkoxy substituted by halogen. C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;

### X is —C(R⁹)═ or —N═;

R⁹ is hydrogen, C₁-C₆ alkoxy, NO₂ or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

In certain embodiments, the TAAR1 inhibitor is N-(3-ethoxy-phenyl)-4-pyrrolidin-1-yl-3-trifluoromethyl-benzamide.

A method for diagnosing irritable bowel syndrome (IBS) in a subject, the method comprising: providing a fecal sample obtained from the subject; determining the amount of one or more markers selected from the group consisting of Ruminococcus gnavus, phenethylamine, tryptamine and tyramine in the fecal sample; and determining based on the amount of the one or more markers in the fecal sample if the subject has IBS.

In certain embodiments, the IBS is a diarrhea-predominant subtype (IBS-D).

In certain embodiments, the one or more markers is phenethylamine and tyramine.

In certain embodiments, the method further comprises the step of administering a therapeutically effective amount of a trace amine-associated receptor 1 (TAAR1) inhibitor to the subject.

In certain embodiments, the TAAR1 inhibitor is N-(3-ethoxy-phenyl)-4-pyrrolidin-1-yl-3-trifluoromethyl-benzamide.

### BRIEF DESCRIPTION OF THE DRAWINGS

The above and other objects and features of the present invention will become apparent from the following description of the invention, when taken in conjunction with the accompanying drawings, in which:

FIG. 1 shows the enrichment of Ruminococcus gnavus in IBS-D patients and its positive correlation with serum 5-HT level and diarrhea symptoms. (A-B) Serum 5-HT and urine 5-HIAA levels were higher in IBS-D patients (n=290) compared with healthy control (HC, n=89) subjects. (C) Spearman rand p-values plot against gut bacteria species abundances and serum 5-HT level in IBS-D patients. (D) Relative abundances of selected gut bacteria species in IBS-D patients and HC subjects. (E-F) Spearman's correlation between relative abundances of R. gnavus with serum 5-HT level and Bristol Stool Scale in IBS-D patients. Differences of relative abundances of R. gnavus in IBS-D patients and HC subjects were analyzed by one-tailed Mann-Whitney tests. Data are presented as mean±S.E.M.

FIG. 2 shows monoassociation of Ruminococcus gnavus induces IBS-D-like symptoms in germ-free mice. (A-B) Serum and luminal 5-HT level in germ-free mice after monoassocation with/without R. gnavus (ATCC 29149) (n=6/group). (C-E) GI transit time, fecal water content and defecation frequency indexes in germ-free mice after monoassocation with/without R. gnavus (ATCC 29149)(n=6/group). (F) Fecal phenethylamine, tryptamine and tyramine level in germ-free mice after monoassocation with/without R. gnavus (ATCC 29149) (n=6/group). Differences of serum and intestine 5-HT level, GI transit time, fecal water content and defecation frequency indexes were analyzed by two-tailed student t-tests. Differences of fecal phenethylamine, tryptamine and tyramine levels in mice were analyzed by one-tailed student t-tests. Data are presented as mean±S.D.

FIG. 3 shows fecal phenethylamine and tryptamine positively correlate with serum 5-HT level and R. gnavus abundances in IBS-D patients. (A-C) phenethylamine, tryptamine and tyramine levels in fecal samples of IBS-D patients (n=290) and HC subjects (n=89). (D-E) Spearman's correlation between fecal phenethylamine level with Bristol Stool Scale and serum 5-HT level in IBS-D subjects. (F-G) Spearman's correlation between fecal tryptamine level with Bristol Stool Scale and serum 5-HT level in IBS-D subjects. (H-I) Distribution of fecal phenethylamine and tryptamine levels in IBS-D patients (n=290) and HC subjects (n=89). Based on the 75th percentile of fecal phenethylamine and tryptamine levels in HC, 44% of IBS-D patients (n=128) were found with excessive fecal phenethylamine and 51% of IBS-D patients (n=148) with found with excessive fecal tryptamine. (J-K) Spearman's correlation between fecal phenethylamine and tryptamine level with R. gnavus abundances in IBS-D subjects. Differences of fecal phenethylamine, tryptamine and tyramine levels in IBS and HC as well as batch culture samples were analyzed by one-tailed Mann-Whitney tests. Data are presented as mean±S.E.M.

FIG. 4 shows Phenethylamine and tryptamine activate 5-HT production, accelerate GI transit and increase colonic secretion in vitro and in vivo. (A-B) 5-HT level in mice ex vivo intestinal tissues after treatment of phenethylamine and tryptamine at the indicated concentrations (25 μM and 50 μM) or control for 2 hours (n=3/group). (C-F) 5-HT level in mice serum and intestinal tissues after treatment of phenethylamine or tryptamine at the indicated doses (2 mg/kg, 5 mg/kg and 10 mg/kg) or control (water) (n=6/group). (G-H) Fecal water content and GI transit time in mice after treatment of phenethylamine at the indicated doses (2 mg/kg, 5 mg/kg and 10 mg/kg) or control (water)(n=6/group). (I-J) 5-HT level in mice serum and intestinal tissues after treatment of phenethylamine (10 mg/kg), TPH1 inhibitor (LX-1031, 50 mg/kg) or control (water)(n=6/group). (K-L) Fecal water content and GI transit time in mice after treatment with phenethylamine (10 mg/kg) and/or TPH1 inhibitor (LX-1031, 50 mg/kg) or control (water) (n=6/group). Differences of 5-HT level, GI transit time and fecal water content were analyzed by t-test (two-tailed Mann-Whitney test) or ordinary one-way ANOVA. Data are presented as mean±S.D.

FIG. 5 shows Phenethylamine stimulates 5-HT production via a TAAR1-dependent mechanism. (A) 5-HT biosynthesis and metabolism profiles in mice after treatment of phenethylamine at the indicated doses (2 mg/kg, 5 mg/kg and 10 mg/kg) or control (water) (n=6/group). (B-C) Western blot (and quantification) in proximal colonic tissues of mice after treatment of phenethylamine at the indicated doses (2 mg/kg, 5 mg/kg and 10 mg/kg) or control (water) (n=3/group). (D-E) Western blot (and quantification) in proximal colonic tissues of mice after treatment of phenethylamine (10 mg/kg) and TAAR1 antagonist EPPTB (10 mg/kg) or control (1% DMSO in saline) (n=3/group). (F-I) GI transit time, fecal water content and serum 5-HT level in mice after treatment of phenethylamine (10 mg/kg) and TAAR1 antagonist EPPTB (10 mg/kg) or/and control (1% DMSO in saline) (n=6/group). 5-HT level in mice colonic tissues after treatment of phenethylamine (501M) or/and TAAR1 antagonist EPPTB (50 μM) or control (1% DMSO) (n=3/group). Differences of 5-HT level, GI transit time and fecal water content were analyzed using ordinary one-way ANOVA. Data are presented as mean±S.D.

FIG. 6 shows In vivo phenethylamine and tryptamine production by IBS-D-associated bacteria enhance 5-HT synthesis and induce diarrhea-like symptoms. (A) Fecal phenethylamine and tryptamine level in antibiotic-treated mice after colonization with E. coli vector control or E. coli TDC⁺ (n=6/group). (B-D) Serum 5-HT level, GI transit time and fecal water content in antibiotic-treated mice after colonization with E. coli vector control or E. coli TDC⁺ (n=6/group). (E-I) Fecal phenethylamine and tryptamine level, serum 5-HT level, GI transit time and fecal water content in antibiotic-treated mice after colonization with R. gnavus (ATCC 29149) and TAAR1 antagonist EPPTB (10 mg/kg) or control (1% DMSO in saline) (n=6/group). (J-L) Colonic 5-HT level, GI transit time and defecation frequency in antibiotic-treated mice after colonization with gut microbiota from IBS-D patients (n=8) and HC subjects (n=8) and TAAR1 antagonist EPPTB (10 mg/kg) or/and control (1% DMSO in saline) (n=6/group). Differences of phenethylamine and tryptamine levels in feces, 5-HT level in serum and intestine, and GI transit time defecation frequency were analyzed using Student's t-test (two-tailed Mann-Whitney test) or ordinary one-way ANOVA. Data are presented as mean±S.D.

FIG. 7 shows Ruminococcus gnavus is positively correlated with serum 5-HT levels and diarrhea symptoms in IBS-D patients, related to FIG. 1. (A-B) Spearman's correlation between relative abundances of R. gnavus with serum 5-HT level and diarrhea frequency in IBS-D patients (n=87).

FIG. 8 shows monoassociation of Ruminococcus gnavus induces IBS-D-like symptoms in germ-free mice, Related to FIG. 2. (A-B) GI transit time and fecal water content indexes in normal mice with intact microbiome after administration with/without R. gnavus (ATCC 29149) (n=6/group). (C) Score plot of the fecal metabolome in germ-free mice after monoassocation with/without R. gnavus (ATCC 29149) (n=6/group). (D) Volcano plot of the fecal metabolome in germ-free mice after monoassocation with/without R. gnavus (ATCC 29149) (n=6/group).

FIG. 9 shows fecal phenethylamine and tryptamine are increased and positively correlated with serum 5-HT levels in IBS-D patients, Related to FIG. 3. (A-C) Phenethylamine, tryptamine and tyramine levels in batch culture samples using feces from IBS-D and HC groups (n=30/group). (D-F) Spearman's correlation between fecal phenethylamine level with R. gnavus abundances, diarrhea frequency and serum 5-HT in IBS-D subjects (n=87). (G-I) Spearman's correlation between fecal tryptamine level with R. gnavus abundances, diarrhea frequency and serum 5-HT in IBS-D subjects (n=87). Differences of fecal tryptamine, Trp, tyramine and tyrosine level in IBS and HC as well as batch culture samples were analyzed by the one-tailed Mann-Whitney test. Data are presented as mean±S.E.M.

FIG. 10 shows phenethylamine and tryptamine activated 5-HT production, Related to FIG. 4. (A) 5-HT level in human colonic tissues after treatment of phenethylamine (50 μM) or tryptamine (50 μM) or control (1% DMSO) (n=6/group). (B-C) 5-HT level in QGP-1 cells after treatment of phenethylamine and tryptamine as indicated concentration (2 μM, 4 μM, 8 μM, 16 μM and 32 μM) or control for 24 hours and 48 hours (n=3/group). (D) 5-HT level in QGP-1 cells after treatment of metabolite and precursor of phenethylamine, phenylacetic acid (PAA), or phenylalanine (Phe) as indicated concentration (25 μM, 50 μM, 100 μM and 200 μM) or control for 24 hours (n=3/group). (E) 5-HT level in QGP-1 cells after treatment of metabolite and precursor of tryptamine, indole acetic acid (IAA), or tryptophan (Trp) as indicated concentration (25 μM, 50 μM, 100 μM and 200 μM) or control for 24 hours (n=3/group). (F-G) Fecal water content and GI transit time in mice after treatment of tryptamine at the indicated doses (2 mg/kg, 5 mg/kg and 10 mg/kg) or control (water) (n=6/group). Differences of 5-HT, fecal water content and GI transit time were analyzed by ordinary one-way ANOVA. Data are presented as mean±S.D.

FIG. 11 shows phenethylamine stimulated 5-HT production via a TAAR1-dependent mechanism, Related to FIG. 5. (A-C) Serum Trp, 5-HTP and 5-HIAA in mice intestinal tissues and serum after treatment of phenethylamine as indicated dosages (2 mg/kg, 5 mg/kg and 10 mg/kg) or control (water) (n=6/group). (D-F) Serum Trp, 5-HTP, 5-HIAA in mice after treatment of phenethylamine (10 mg/kg) and TAAR1 antagonist EPPTB (10 mg/kg) or control (1% DMSO in saline) (n=6/group). (G) 5-HT biosynthesis and metabolism profiles (expressed by relative fold changes) in mice after treatment of phenethylamine (10 mg/kg) and TAAR1 antagonist EPPTB (10 mg/kg) or control (1% DMSO in saline) (n=6/group). (H) Genotyping and qPCR results of Taar1 expression in enterochromaffin (EC) cells and non-EC intestinal cells from Taar1^{-/-} mice and wild-type littermates. (I-L) Fecal water content, defecation frequency, GI transit time and serum 5-HT level in Taar1^{-/-} mice and wild-type littermates after treatment of phenethylamine (10 mg/kg) or/and control (1% DMSO in saline) (n=6/group). (M) 5-HT level in QGP-1 cells after treatment of phenethylamine, tryptamine and 5-HTR4 antagonist (Piboserod) as indicated concentration or control for 24 hours (n=3/group). Differences of 5-HT biosynthesis and metabolism profiles in mice serum and QGP-1 cells, fecal water content, defecation frequency and GI transit time were analyzed by ordinary one-way ANOVA. Data are presented as mean±S.D.

FIG. 12 shows In vivo phenethylamine and tryptamine production by IBS-D-associated bacteria enhanced 5-HT synthesis and induced diarrhea-like symptoms, Related to FIG. 6. (A) PCR electrophoresis of the positive clone of E. coli with TDC gene. (B-C) LC-MS chromatogram of phenethylamine and tryptamine level in LB culture medium of E. coli TDC+ and E. coli vector control. (D) Relative abundances of total bacteria (16s rDNA) in antibiotic-treated mice after colonization with E. coli vector control or E. coli TDC⁺. (E) Relative abundances of R. gnavus and total bacteria (16s DNA) in antibiotic-treated mice after colonization with R. gnavus (ATCC 29149). (F-H) Serum Trp, 5-HTP and 5-HIAA levels in antibiotic-treated mice after colonization with R. gnavus (ATCC 29149) and TAAR1 antagonist EPPTB (10 mg/kg) or control (1% DMSO in saline) (n=6/group). Differences of 5-HT biosynthesis and metabolism profiles in mice serum were analyzed by ordinary one-way ANOVA. Data are presented as mean±S.D.

FIG. 13 shows correlation between serum 5-HT with gut bacteria species in IBS-D patients.

FIG. 14 shows correlation between fecal phenethylamine (PEA) with gut bacteria species in IBS-D patients.

FIG. 15 shows protein blast alignments used to identify potential tryptamine/phenethylamine producers.

### DETAILED DESCRIPTION

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

As used herein, the term “selectively binds TAAR1” refers to a TAAR1 inhibitor to that preferably has an IC50 value for TAAR1 that is at least 2-fold lower than its IC50 value for other TAAR members (e.g., human TAAR2-6 or mouse TAAR2-16). In certain embodiments, the TAAR1 inhibitor has an IC50 value for TAAR1 that is at least 3-fold lower, at least 4-fold lower, at least 5-fold lower, at least 10-fold lower, at least 15-fold lower, at least 20-fold lower, at least 30-fold lower, at least 40-fold lower, at least 50-fold lower, at least 100-fold lower, at least 500-fold lower, or at least 1,000-fold lower than its IC50 value for other TAAR members.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein.

The term “therapeutically effective amount” as used herein, means that amount of the compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

Provided herein is a method of treating irritable bowel syndrome (IBS) in a subject in need thereof, the method comprising: administering a therapeutically effective amount of a trace amine-associated receptor 1 (TAAR1) inhibitor to the subject.

IBS can be categorized four basic subtypes: diarrhea-predominant subtype (IBS-D), constipation-predominant subtype (IBS-C), mixed bowel subtype (IBS-M), and unclassified subtype (IBS-U). The IBS can be any subtype. In certain embodiments, the IBS is a diarrhea-predominant subtype (IBS-D).

The TAAR1 inhibitor can be a small molecule, antibody, protein, or the like. In certain embodiments, the TAAR1 inhibitor is a small molecule.

US2009/0036420A1, which is hereby incorporated by reference in its entirety, discloses the use of a TAAR1 inhibitor for treating a CNS disorder, wherein the TAAR1 inhibitor is a compound of Formula 1:

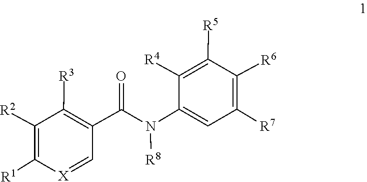

or a pharmaceutically acceptable salt thereof, wherein
n is 0, 1, 2 or 3,
p is 0 or 1;
R¹ is hydrogen, halogen, C₁-C₆ alkyl, C₁-C₆alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆alkyl-O—C(═CH₂)—, —C(O)—C₁-C₆alkyl, tetrahydro-furan-2-yl, morpholin-4-yl, pyrazol-1-yl, or —OC(O)—C₁-C₆ alkyl, or R¹ and R² together with the corresponding C-atoms form a ring comprising —CH═CH—CH═CH— or —S—(CH₂)₄—;
R³ is hydrogen, halogen, C₁-C₆ alkyl or C₁-C₆alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—,
R⁸ is hydrogen or C₁-C₆ alkyl;
X⁸ is —C(R⁹)═ or —N═;
R⁹ is hydrogen, C₁-C₆ alkoxy, NO₂ or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen. C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

In certain embodiments, the TAAR1 inhibitor is selected from the group consisting of N-(3-methoxy-phenyl)-4-(4-methyl-piperidin-1-yl)-3-nitro-benzamide, N-(3-methoxy-phenyl)-3-nitro-4-propylamino-benzamide, 4-benzylamino-N-(3-methoxy-phenyl)-3-nitro-benzamide, 4-ethylamino-N-(3-methoxy-phenyl)-3-nitro-benzamide, 4-isopropylamino-N-(3-methoxy-phenyl)-3-nitro-benzamide, 4-azetidin-1-yl-N-(3-methoxy-phenyl)-3-nitro-benzamide, N-(3-methoxy-phenyl)-3-nitro-4-pyrrolidin-1-yl-benzamide, N-(3-methoxy-phenyl)-3-nitro-4-piperidin-1-yl-benzamide, N-(3-methoxy-phenyl)-3-nitro-4-phenylamino-benzamide, N-(3-methoxy-phenyl)-4-pyrrolidin-1-yl-3-trifluoromethyl-benzamide, 4-(2-methoxy-ethylamino)-N-(3-methoxy-phenyl)-3-trifluoromethyl-benzamide, 4-azetidin-1-yl-N-(3-methoxy-phenyl)-3-trifluoromethyl-benzamide, N-(3-methoxy-phenyl)-4-piperidin-1-yl-3-trifluoromethyl-benzamide, N-(3-methoxy-phenyl)-4-(4-methyl-piperidin-1-yl)-3-trifluoromethyl-benzamide, N-(3-methoxy-phenyl)-4-propylamino-3-trifluoromethyl-benzamide, 4-butylamino-N-(3-methoxy-phenyl)-3-trifluoromethyl-benzamide, 4-benzylamino-N-(3-methoxy-phenyl)-3-trifluoromethyl-benzamide, 4-ethylamino-N-(3-methoxy-phenyl)-3-trifluoromethyl-benzamide, 4-isopropylamino-N-(3-methoxy-phenyl)-3-trifluoromethyl-benzamide, N-(3-methoxy-phenyl)-4-morpholin-4-yl-3-trifluoromethyl-benzamide, N-(3-ethyl-phenyl)-4-pyrrolidin-1-yl-3-trifluoromethyl-benzamide, N-(3-ethoxy-phenyl)-4-pyrrolidin-1-yl-3-trifluoromethyl-benzamide, N-(3-isopropyl-phenyl)-4-pyrrolidin-1-yl-3-trifluoromethyl-benzamide, N-(3-isopropoxy-phenyl)-4-pyrrolidin-1-yl-3-trifluoromethyl-benzamide, N-(3-acetyl-phenyl)-4-pyrrolidin-1-yl-3-trifluoromethyl-benzamide, N-(3-fluoro-phenyl)-4-pyrrolidin-1-yl-3-trifluoromethyl-benzamide, N-(3-chloro-phenyl)-4-pyrrolidin-1-yl-3-trifluoromethyl-benzamide, N-(3-bromo-phenyl)-4-pyrrolidin-1-yl-3-trifluoromethyl-benzamide, 4-pyrrolidin-1-yl-N-m-tolyl-3-trifluoromethyl-benzamide, N-(3-difluoromethoxy-phenyl)-4-pyrrolidin-1-yl-3-trifluoromethyl-benzamide, 4-pyrrolidin-1-yl-N-[3-(1,1,2,2-tetrafluoro-ethoxy)-phenyl]-3-trifluoromethyl-benzamide, (rac,meso)-4-(3,5-dimethyl-piperidin-1-yl)-N-(3-methoxy-phenyl)-3-trifluoromethyl-benzamide, 4-azepan-1-yl-N-(3-methoxy-phenyl)-3-trifluoromethyl-benzamide, 4-(4-cyano-piperidin-1-yl)-N-(3-methoxy-phenyl)-3-trifluoromethyl-benzamide, N-(3-methoxy-phenyl)-3-trifluoromethyl-4-(4-trifluoromethyl-piperidin-1-yl)-benzamide, N-(3-chloro-phenyl)-6-piperazin-1-yl-nicotinamide, N-(3-chloro-phenyl)-6-(4-methyl-piperazin-1-yl)-nicotinamide, 5-chloro-N-(3-chloro-phenyl)-6-methylamino-nicotinamide, 5-chloro-N-(3-chloro-phenyl)-6-isopropylamino-nicotinamide, 5-chloro-N-(3-chloro-phenyl)-6-(2-methoxy-ethylamino)-nicotinamide, 5-chloro-N-(3-chloro-phenyl)-6-pyrrolidin-1-yl-nicotinamide, 3′-chloro-4-methyl-3,4,5,6-tetrahydro-2H-[1,2′]bipyridinyl-5′-carboxylic acid (3-chloro-phenyl)-amide, 5-chloro-N-(3-chloro-phenyl)-6-ethylamino-nicotinamide, 5-chloro-N-(3-chloro-phenyl)-6-propylamino-nicotinamide, 6-butylamino-5-chloro-N-(3-chloro-phenyl)-nicotinamide, 6-azetidin-1-yl-5-chloro-N-(3-chloro-phenyl)-nicotinamide, 3′-chloro-3,4,5,6-tetrahydro-2H-[1,2′]bipyridinyl-5′-carboxylic acid (3-chloro-phenyl)amide, 5-chloro-N-(3-chloro-phenyl)-6-(4-methyl-piperazin-1-yl)-nicotinamide, N-(3-methoxy-phenyl)-6-pyrrolidin-1-yl-5-trifluoromethyl-nicotinamide, 6-benzylamino-N-(3-methoxy-phenyl)-5-trifluoromethyl-nicotinamide, 6-isopropylamino-N-(3-methoxy-phenyl)-5-trifluoromethyl-nicotinamide, 4-methyl-3′-trifluoromethyl-3,4,5,6-tetrahydro-2H-[1,2′]bipyridinyl-5′-carboxylic acid (3-methoxy-phenyl)-amide, 5-chloro-N-(3-methoxy-phenyl)-6-pyrrolidin-1-yl-nicotinamide, 3′-chloro-4-methyl-3,4,5,6-tetrahydro-2H-[1,2′]bipyridinyl-5′-carboxylic acid (3-methoxy-phenyl)-amide, 6-butylamino-5-chloro-N-(3-methoxy-phenyl)-nicotinamide, 5-chloro-N-(3-chloro-phenyl)-6-piperazin-1-yl-nicotinamide, 4-chloro-N-phenyl-3-trifluoromethyl-benzamide, 4-chloro-N-(3-methoxy-phenyl)-3-nitro-benzamide, 4-bromo-N-(3-methoxy-phenyl)-3-nitro-benzamide, 3-chloro-4-fluoro-N-(3-methoxy-phenyl)-benzamide, 3-bromo-4-fluoro-N-(3-methoxy-phenyl)-benzamide, 4-fluoro-N-(3-methoxy-phenyl)-3-trifluoromethyl-benzamide, 4-fluoro-N-(3-methoxy-phenyl)-3-nitro-benzamide, 3,4-dichloro-N-[3-(2,5-dimethyl-imidazol-1-ylmethyl)-phenyl]-benzamide, 3,4-dichloro-N-phenyl-benzamide, 4-chloro-N-(3-methoxy-phenyl)-3-trifluoromethyl-benzamide, 3,4-dichloro-N-phenyl-benzamide, 3,3′,4-trichlorobenzanilide, 3,4-dichloro-N-(3-chloro-phenyl)-benzamide, 5,6-dichloro-N-(3-chloro-phenyl)-nicotinamide, 5,6-dichloro-N-(3-methoxy-phenyl)-nicotinamide, 6-chloro-N-(3-methoxy-phenyl)-5-trifluoromethyl-nicotinamide, N-(3-methoxy-phenyl)-3-nitro-4-propylamino-benzamide, 4-benzylamino-N-(3-methoxy-phenyl)-3-nitro-benzamide, 4-ethylamino-N-(3-methoxy-phenyl)-3-nitro-benzamide, 4-isopropylamino-N-(3-methoxy-phenyl)-3-nitro-benzamide, N-(3-methoxy-phenyl)-3-nitro-4-phenylamino-benzamide, 4-(2-methoxy-ethylamino)-N-(3-methoxy-phenyl)-3-trifluoromethyl-benzamide, N-(3-methoxy-phenyl)-4-propylamino-3-trifluoromethyl-benzamide, 4-butylamino-N-(3-methoxy-phenyl)-3-trifluoromethyl-benzamide, 4-benzylamino-N-(3-methoxy-phenyl)-3-trifluoromethyl-benzamide, 4-ethylamino-N-(3-methoxy-phenyl)-3-trifluoromethyl-benzamide, 4-isopropylamino-N-(3-methoxy-phenyl)-3-trifluoromethyl-benzamide, 6-benzylamino-N-(3-methoxy-phenyl)-5-trifluoromethyl-nicotinamide, 6-isopropylamino-N-(3-methoxy-phenyl)-5-trifluoromethyl-nicotinamide, 6-butylamino-5-chloro-N-(3-methoxy-phenyl)-nicotinamide, N-(3-methoxy-phenyl)-4-(4-methyl-piperidin-1-yl)-3-nitro-benzamide, 4-azetidin-1-yl-N-(3-methoxy-phenyl)-3-nitro-benzamide, N-(3-methoxy-phenyl)-3-nitro-4-pyrrolidin-1-yl-benzamide, N-(3-methoxy-phenyl)-3-nitro-4-piperidin-1-yl-benzamide, N-(3-methoxy-phenyl)-4-pyrrolidin-1-yl-3-trifluoromethyl-benzamide, 4-azetidin-1-yl-N-(3-methoxy-phenyl)-3-trifluoromethyl-benzamide, N-(3-methoxy-phenyl)-4-piperidin-1-yl-3-trifluoromethyl-benzamide, N-(3-methoxy-phenyl)-4-(4-methyl-piperidin-1-yl)-3-trifluoromethyl-benzamide, N-(3-methoxy-phenyl)-4-morpholin-4-yl-3-trifluoromethyl-benzamide, N-(3-ethyl-phenyl)-4-pyrrolidin-1-yl-3-trifluoromethyl-benzamide, N-(3-ethoxy-phenyl)-4-pyrrolidin-1-yl-3-trifluoromethyl-benzamide, N-(3-isopropyl-phenyl)-4-pyrrolidin-1-yl-3-trifluoromethyl-benzamide, N-(3-isopropoxy-phenyl)-4-pyrrolidin-1-yl-3-trifluoromethyl-benzamide, N-(3-acetyl-phenyl)-4-pyrrolidin-1-yl-3-trifluoromethyl-benzamide, N-(3-fluoro-phenyl)-4-pyrrolidin-1-yl-3-trifluoromethyl-benzamide, N-(3-chloro-phenyl)-4-pyrrolidin-1-yl-3-trifluoromethyl-benzamide, N-(3-bromo-phenyl)-4-pyrrolidin-1-yl-3-trifluoromethyl-benzamide, 4-pyrrolidin-1-yl-N-m-tolyl-3-trifluoromethyl-benzamide, N-(3-difluoromethoxy-phenyl)-4-pyrrolidin-1-yl-3-trifluoromethyl-benzamide, 4-pyrrolidin-1-yl-N-[3-(1,1,2,2-tetrafluoro-ethoxy)-phenyl]-3-trifluoromethyl-benzamide, (rac,meso)-4-(3,5-dimethyl-piperidin-1-yl)-N-(3-methoxy-phenyl)-3-trifluoromethyl-benzamide, 4-azepan-1-yl-N-(3-methoxy-phenyl)-3-trifluoromethyl-benzamide, 4-(4-cyano-piperidin-1-yl)-N-(3-methoxy-phenyl)-3-trifluoromethyl-benzamide, N-(3-methoxy-phenyl)-3-trifluoromethyl-4-(4-trifluoromethyl-piperidin-1-yl)-benzamide, 4-methyl-3′-trifluoromethyl-3,4,5,6-tetrahydro-2H-[1,2′]bipyridinyl-5′-carboxylic acid (3-methoxy-phenyl)-amide, 5-chloro-N-(3-methoxy-phenyl)-6-pyrrolidin-1-yl-nicotinamide, 3′-chloro-3,4,5,6-tetrahydro-2H-[1,2′]bipyridinyl-5′-carboxylic acid (3-methoxy-phenyl)-amide, 3′-chloro-4-methyl-3,4,5,6-tetrahydro-2H-[1,2′]bipyridinyl-5′-carboxylic acid (3-methoxy-phenyl)-amide, and 5-chloro-N-(3-chloro-phenyl)-6-piperazin-1-yl-nicotinamide; or the TAAR1 inhibitor is selected from the group consisting of:

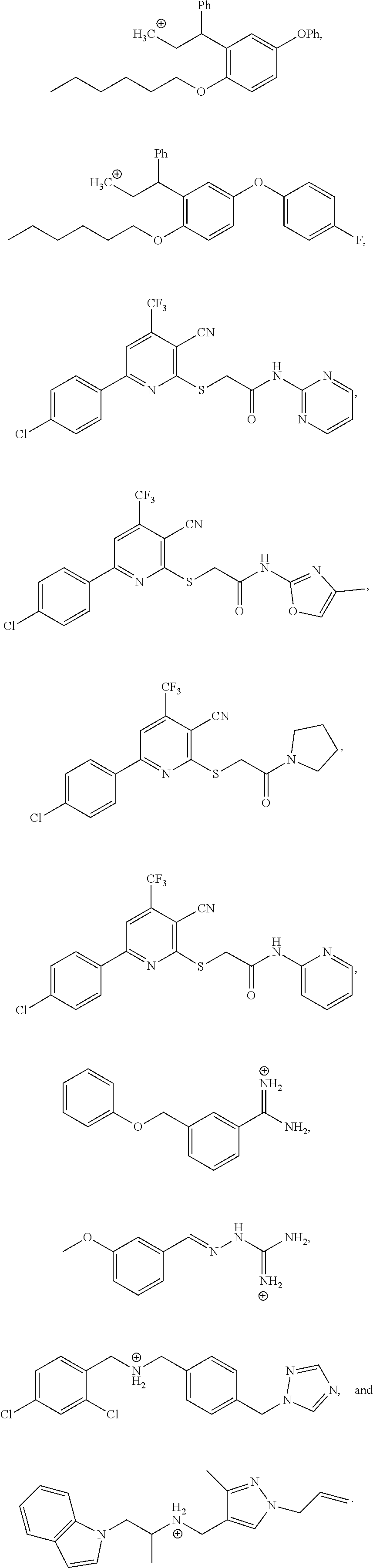

In certain embodiments, the TAAR1 inhibitor is N-(3-ethoxy-phenyl)-4-pyrrolidin-1-yl-3-trifluoromethyl-benzamide (EPPTB).

The subject typically refers to humans, but also to other animals, including, e.g., non-human primates, canines, equines, felines, ovines, porcines, rodents, and the like. In certain embodiments, the subject is a human.

In certain embodiments, a fecal sample obtained from the subject comprises a higher amount of one or more markers selected from the group consisting of Ruminococcus gnavus, phenethylamine, tryptamine and tyramine than an average of amount of the one or more markers in fecal samples obtained from healthy controls (e.g., subjects not suffering from IBS).

In certain embodiments, the method further comprises providing a fecal sample obtained from the subject; determining the amount of one or more markers selected from the group consisting of phenethylamine, tryptamine and tyramine in the fecal sample; and determining based on the amount of the one or more markers in the fecal sample that the subject has IBS prior to the step of administering the TAAR1 inhibitor to the subject.

In certain embodiments, the one or more markers is phenethylamine and tyramine; or phenethylamine, tyramine, and Ruminococcus gnavus.

The step of determining based on the amount of the one or more markers in the fecal sample that the subject has IBS can comprise comparing the measured amount of the one or more markers in the fecal sample with the average amount of the one or more markers in fecal samples obtained from healthy controls (e.g., subjects not suffering from IBS).

The one or more markers can be measured using any method known to those skilled in the art. In certain embodiments, the step of measuring the one or more markers can involve the use of one or more analytical tools, such as high performance liquid chromatography (LC), ultra-performance liquid chromatography (UPLC), liquid chromatography mass spectrometry (LCMS), liquid chromatography tandem mass spectrometry (LCMS/MS), microscopy, gram staining, examining the morphology of bacteria, nucleic acid analysis, e.g., using restriction enzymes, hybridization, polymerase chain reaction (PCR) amplification and/or sequencing, culture-based screening for nutrient requirements and/or antimicrobial sensitivity, analyzing fatty acid distribution, and/or test antigens, or combinations thereof.

The present disclosure also provides a method of treating diarrhea-predominant irritable bowel syndrome (IBS-D) in a subject in need thereof, the method comprising: providing a fecal sample obtained from the subject; determining the amount of one or more markers selected from the group consisting of Ruminococcus gnavus, phenethylamine, tryptamine and tyramine in the fecal sample; determining based on the amount of the one or more markers in the fecal sample that the subject has IBS; and administering a therapeutically effective amount of a trace amine-associated receptor 1 (TAAR1) inhibitor to the subject. In certain embodiments, the TAAR1 inhibitor is any compound described herein. In certain embodiments, the TAAR1 inhibitor is N-(3-ethoxy-phenyl)-4-pyrrolidin-1-yl-3-trifluoromethyl-benzamide.

The present disclosure also provides a method for diagnosing irritable bowel syndrome (IBS) in a subject, the method comprising: providing a fecal sample obtained from the patient; determining the amount of one or more markers selected from the group consisting of Ruminococcus gnavus, phenethylamine, tryptamine, and tyramine in the fecal sample; determining based on the amount of the one or more markers in the fecal sample if the subject has IBS.

In certain embodiments, the IBS is a diarrhea-predominant subtype (IBS-D).

In certain embodiments, the one or more markers is phenethylamine and tyramine; or phenethylamine, tyramine, and Ruminococcus gnavus.

In certain embodiments, the method for diagnosis further comprises administering a therapeutically effective amount of a trace amine-associated receptor 1 (TAAR1) inhibitor to the subject if the subject is determined to suffer from IBS. The TAAR1 inhibitor can be any compound described herein. In certain embodiments, the TAAR1 inhibitor is N-(3-ethoxy-phenyl)-4-pyrrolidin-1-yl-3-trifluoromethyl-benzamide.

Monoassociation with R. gnavus Stimulated Serotonin Production and Induced Diarrhea-Like Symptoms Accompanied by Phenethylamine and Tryptamine Production

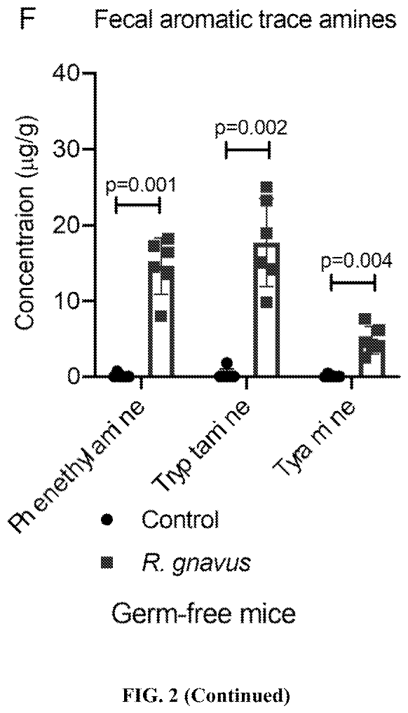

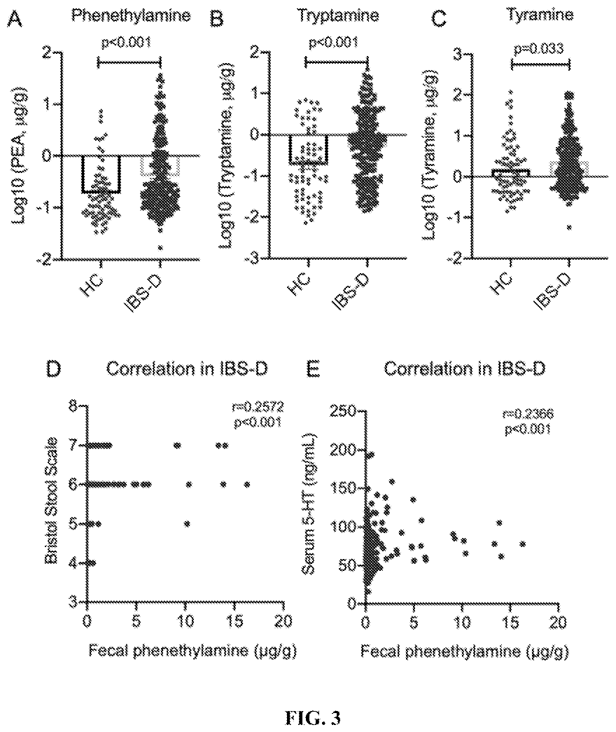

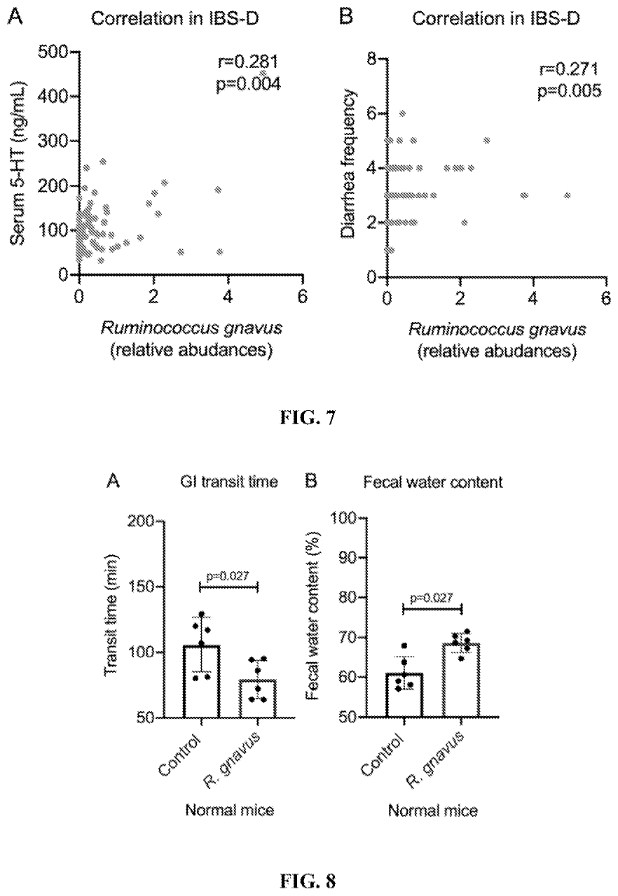

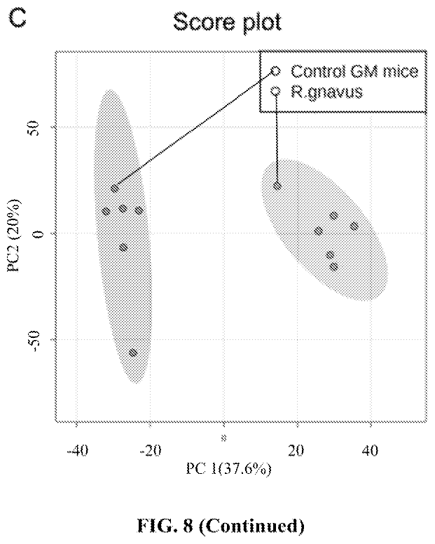

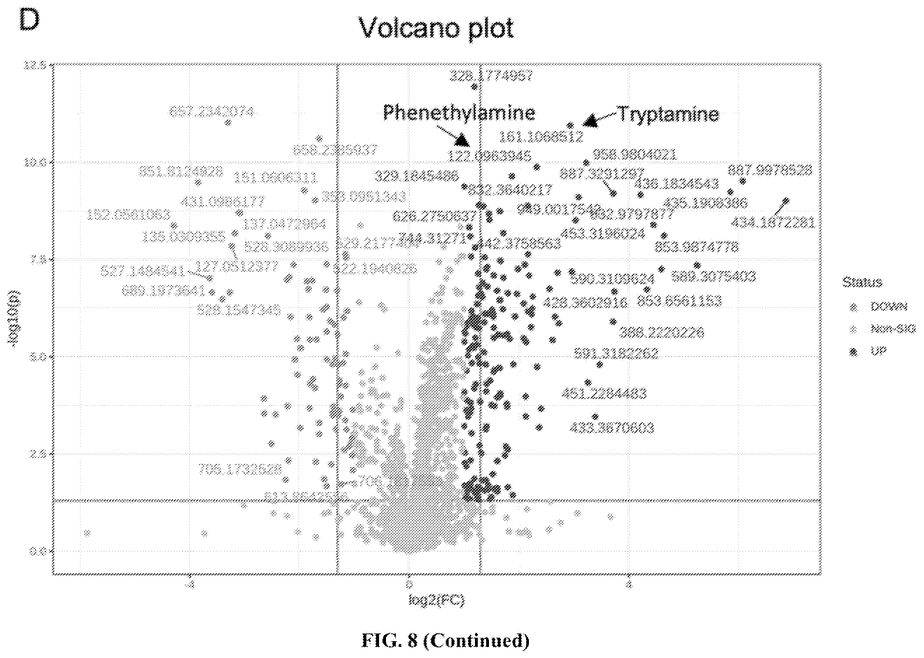

To investigate the potential pathogenic role of R. gnavus in dysregulated 5-HT production, germ-free mice and R. gnavus (strain ATCC 29149) were mono-associated. Germ-free mice mono-associated with R. gnavus exhibited significantly elevated levels of serum 5-HT (p=0.01, FIG. 2A) and luminal 5-HT (p<0.001, FIG. 2B). In these animals, the characteristic features of IBS-D including accelerated GI transit time, increased fecal water content and elevated defecation frequency were observed (p<0.05 for all cases, respectively, FIG. 2C-E). Furthermore, we showed that oral gavage of R. gnavus induced IBS-D-like phenotypes in mice with an intact microbiome (FIG. 8A-B), revealing the potential pathogenic role of R. gnavus in the development of diarrheal symptoms in IBS-D.

To determine the molecular mechanisms underlying the microbial regulation of 5-HT production by R. gnavus, untargeted metabolomic studies were performed to identify changes in the fecal metabolome in germ-free mice with/without monoassociation with R. gnavus (ATCC 29149). Score and volcano plots showed dramatic differences in the metabolic profiles of fecal samples between the two groups (FIG. 8C-D). Notably, a significant elevation of aromatic trace amines, including phenethylamine, tryptamine, and tyramine, was detected in fecal samples of germ-free mice colonized with R. gnavus (p<0.001 for both, FIG. 2F). These data indicate that aromatic trace amines are likely involved in the stimulatory effects of R. gnavus on 5-HT production and GI transit.

### The Positive Association Between Phenethylamine/Tryptamine and Peripheral Serotonin in IBS-D

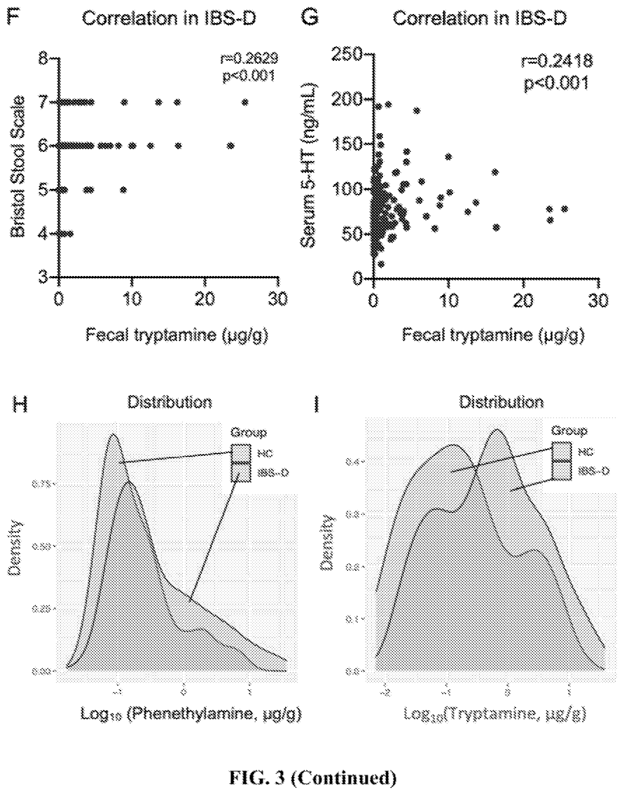

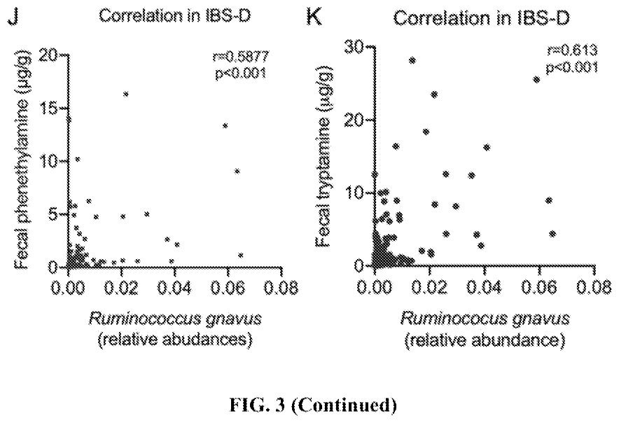

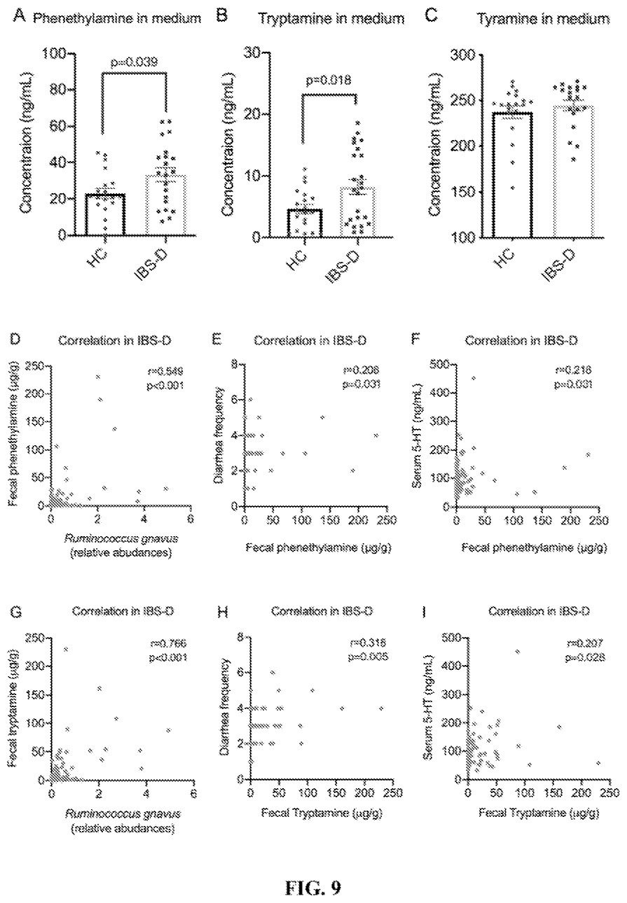

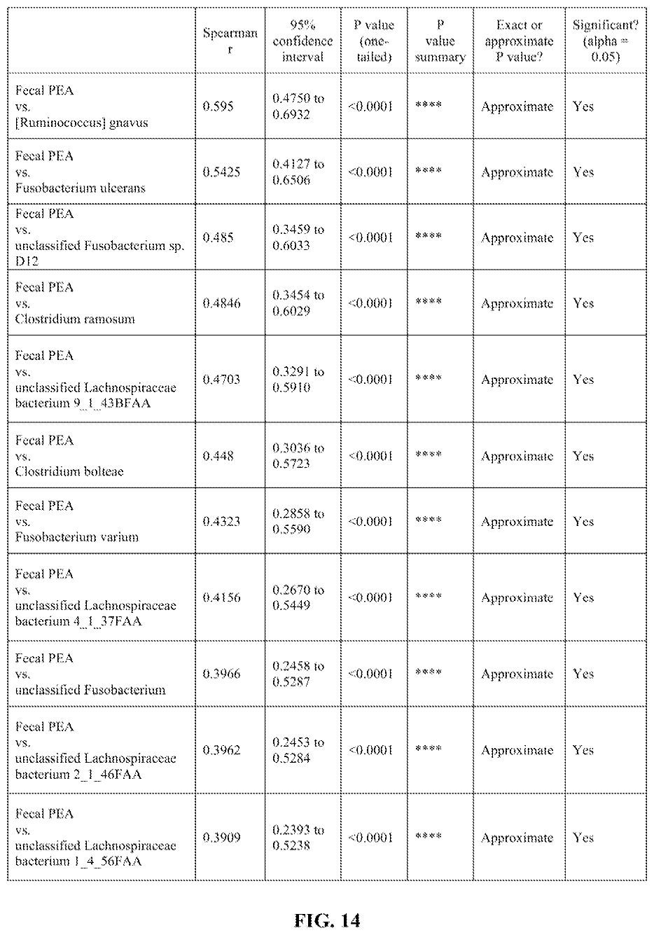

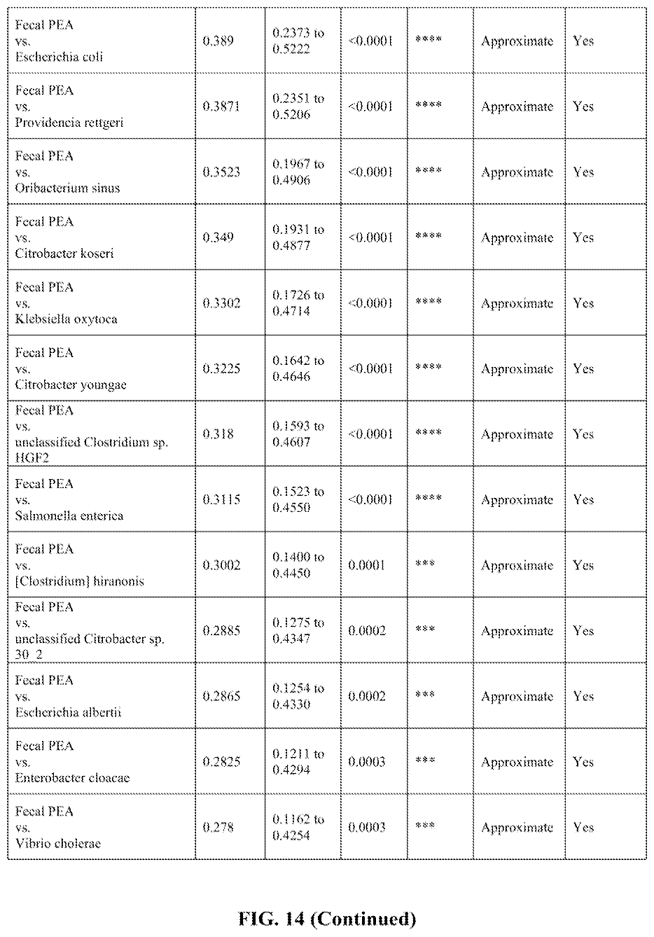

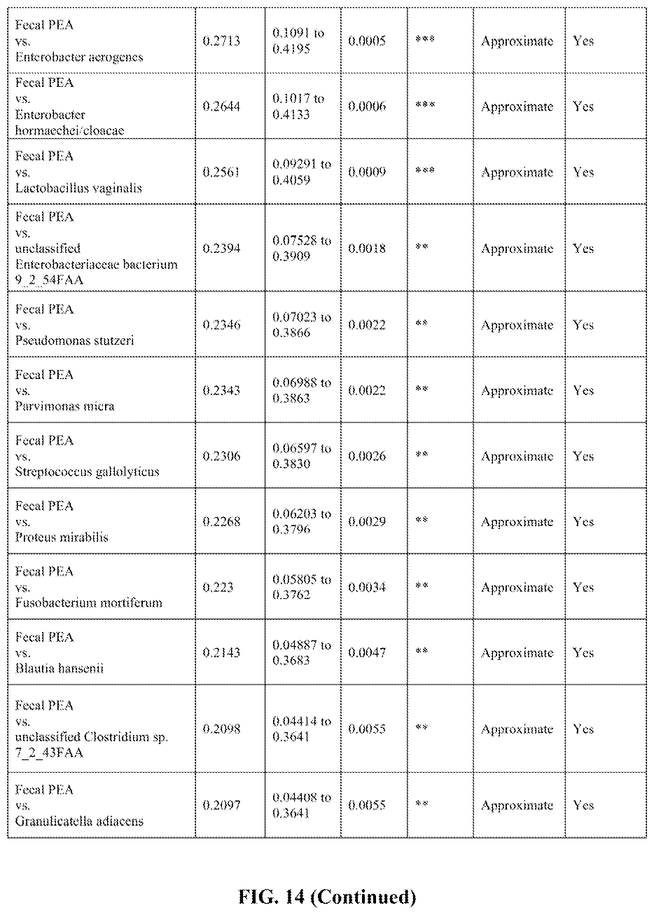

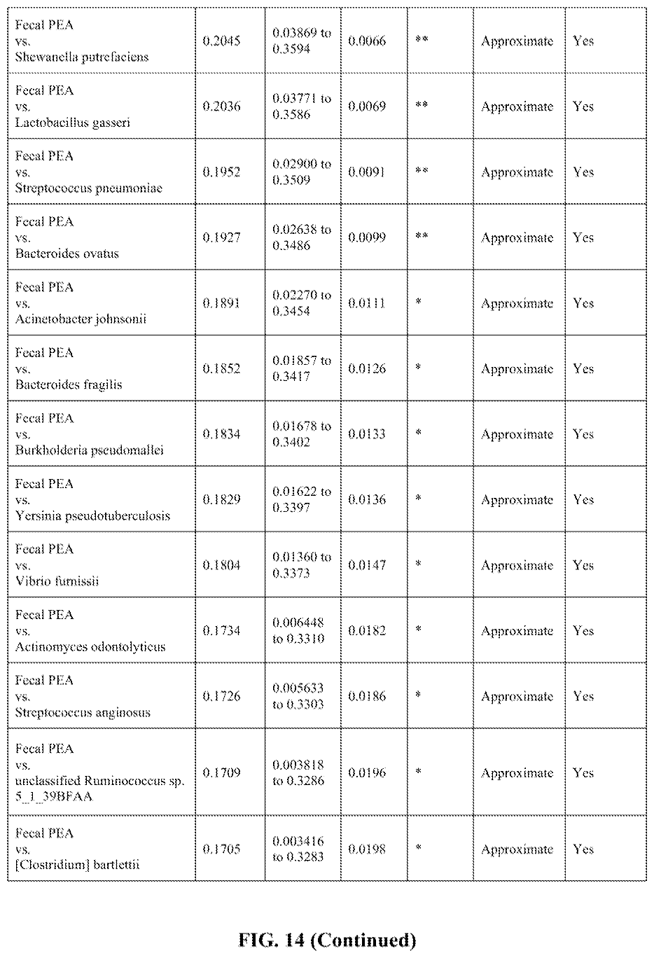

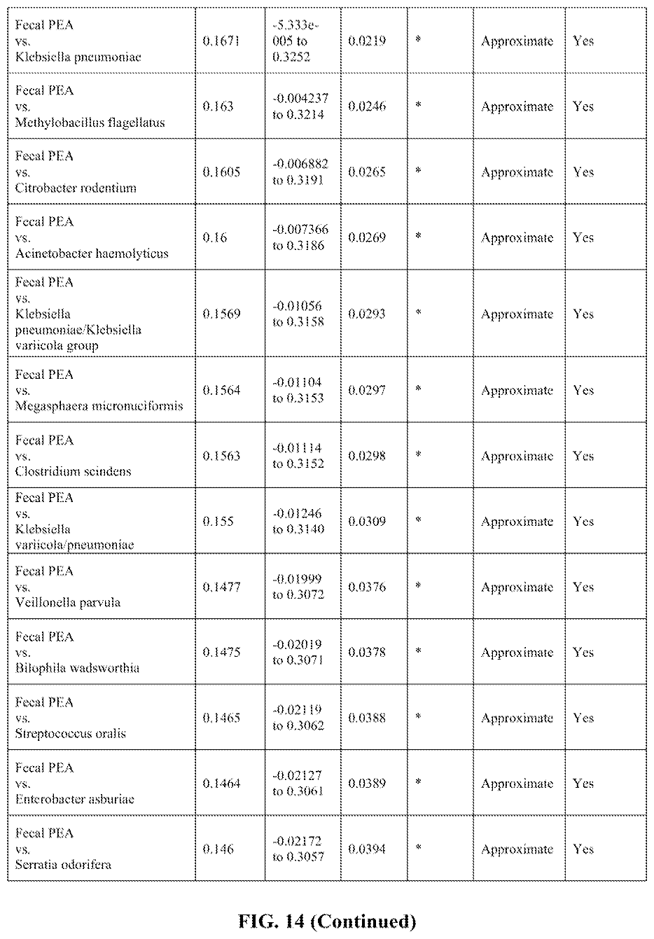

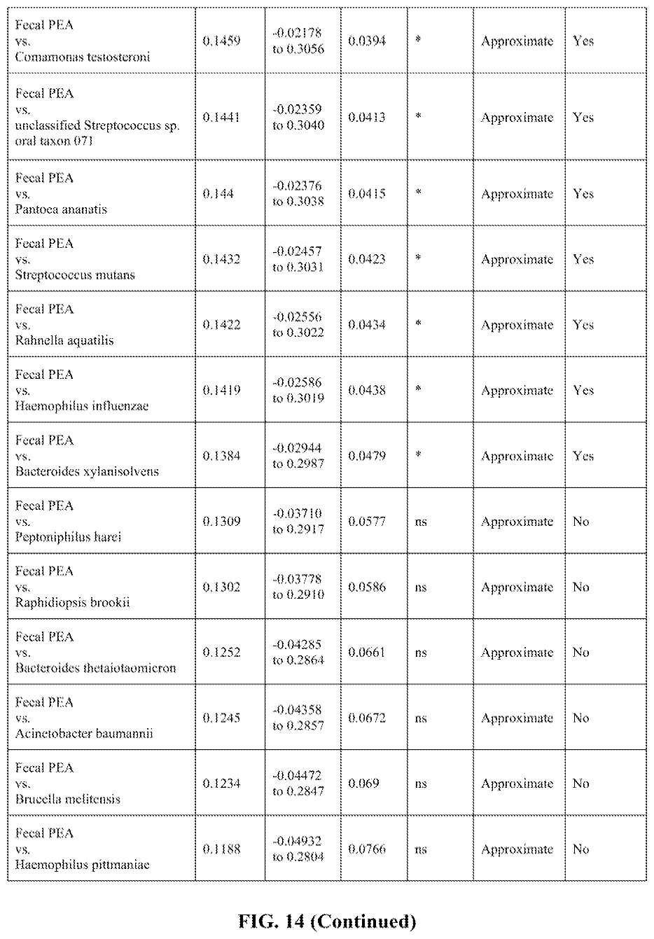

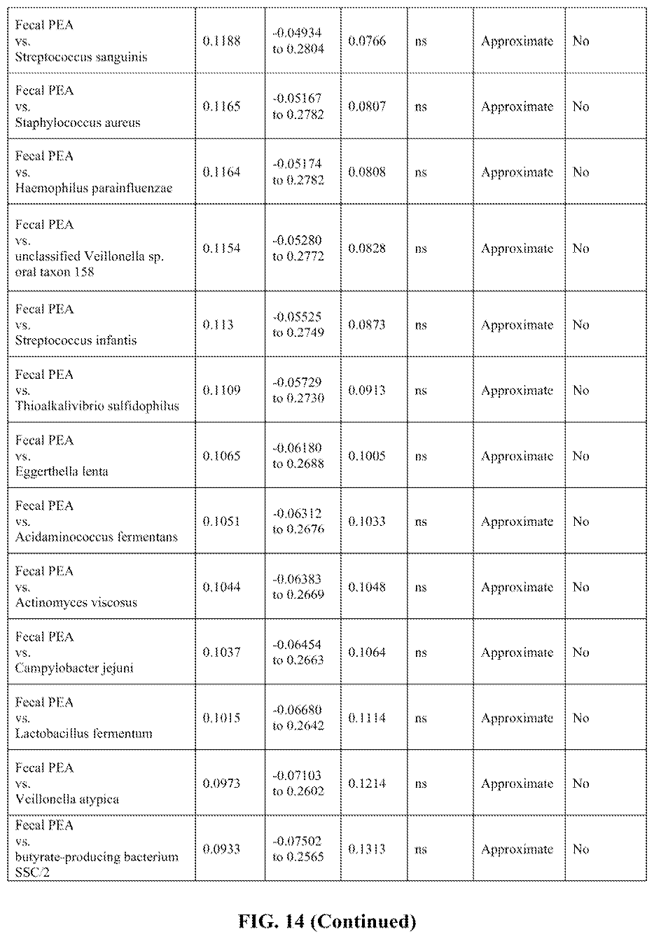

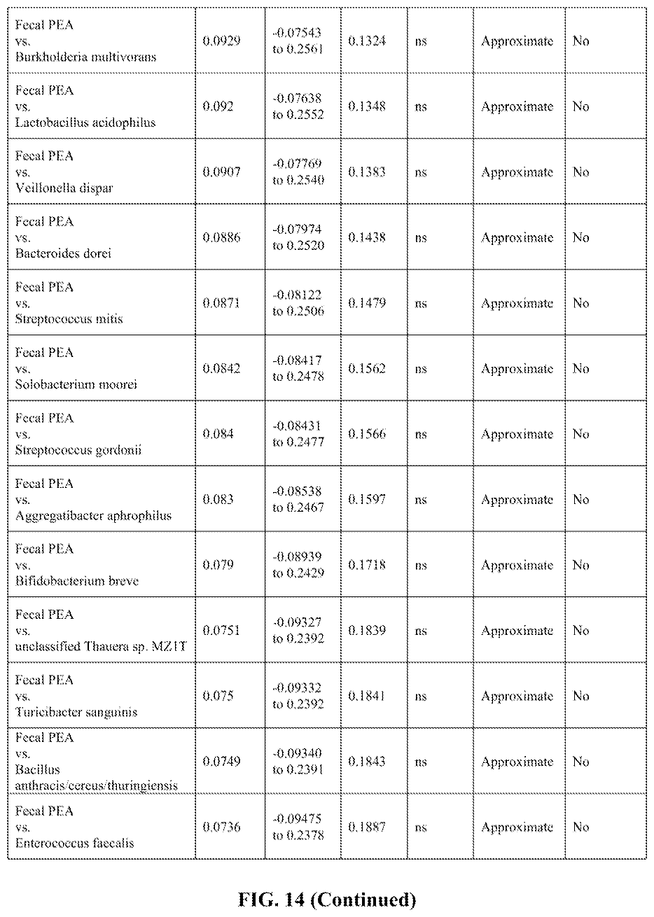

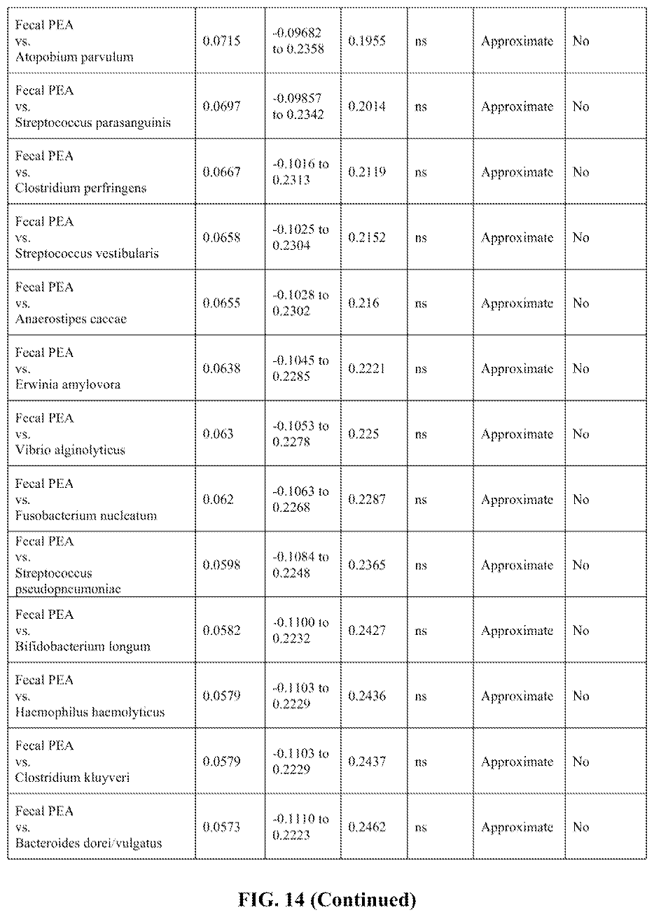

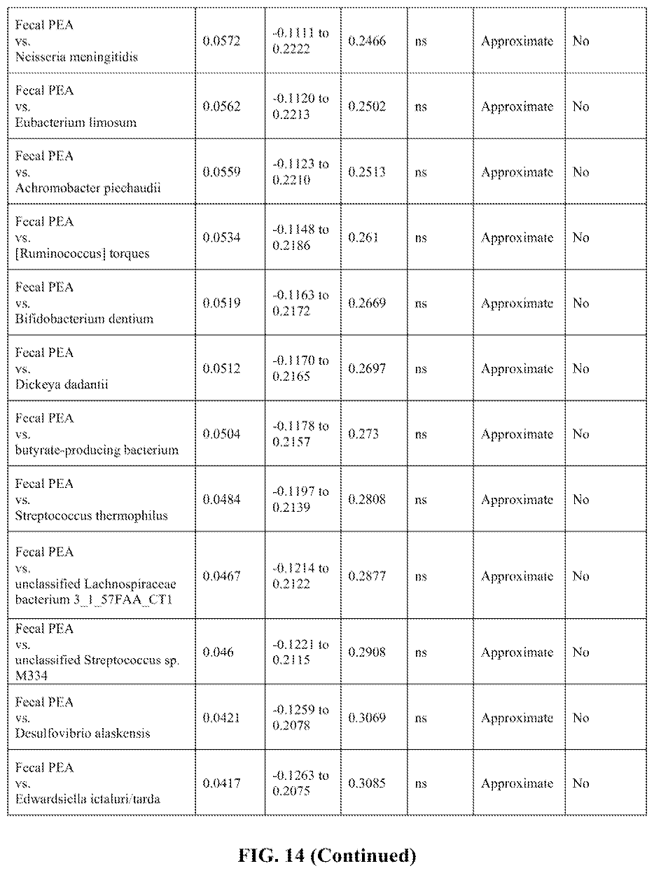

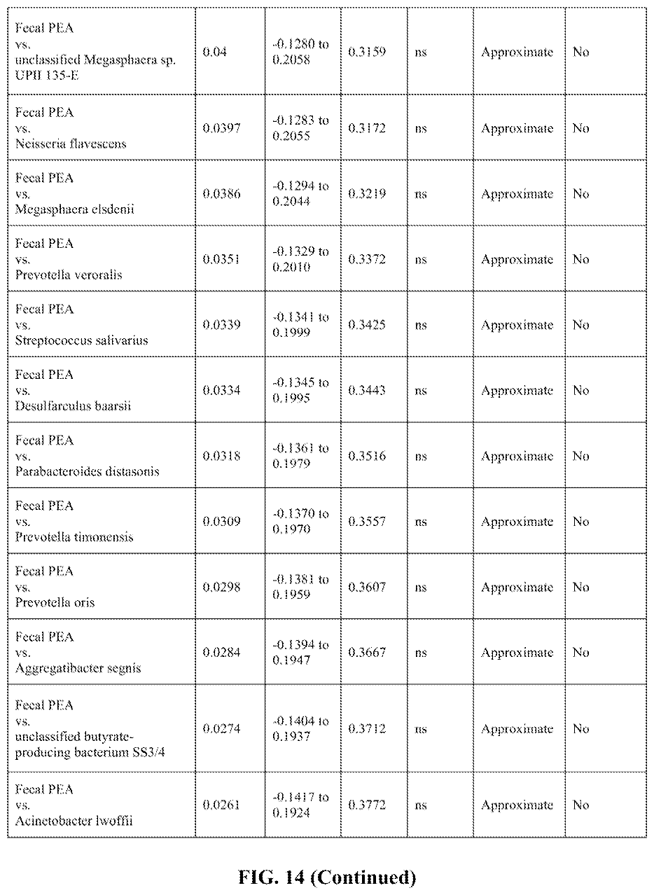

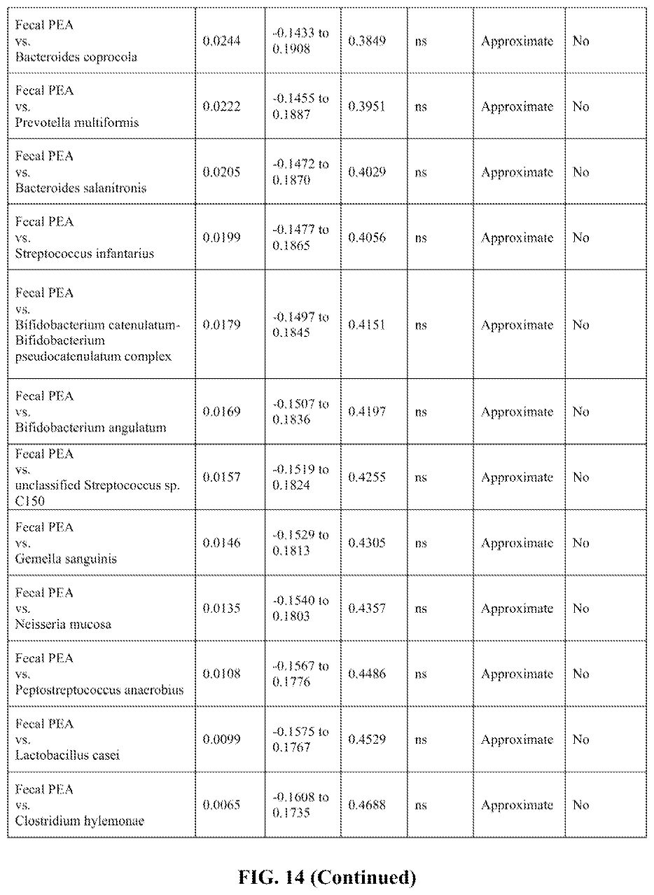

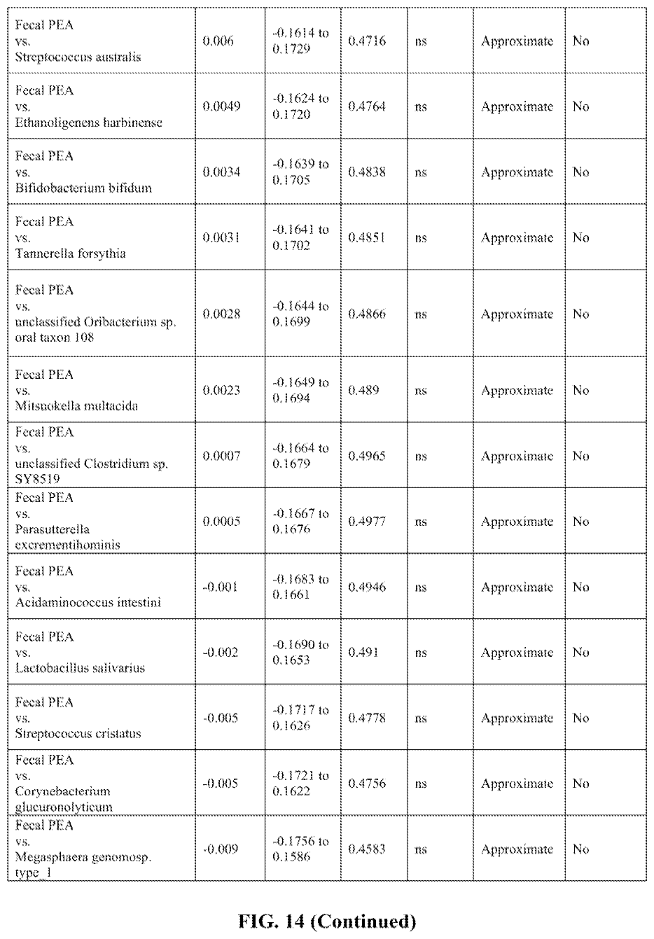

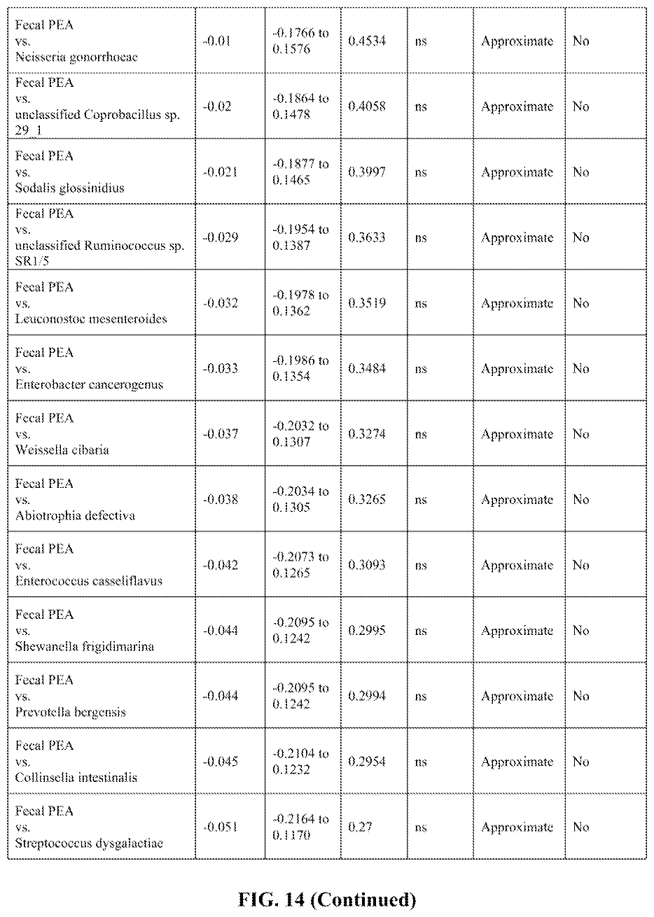

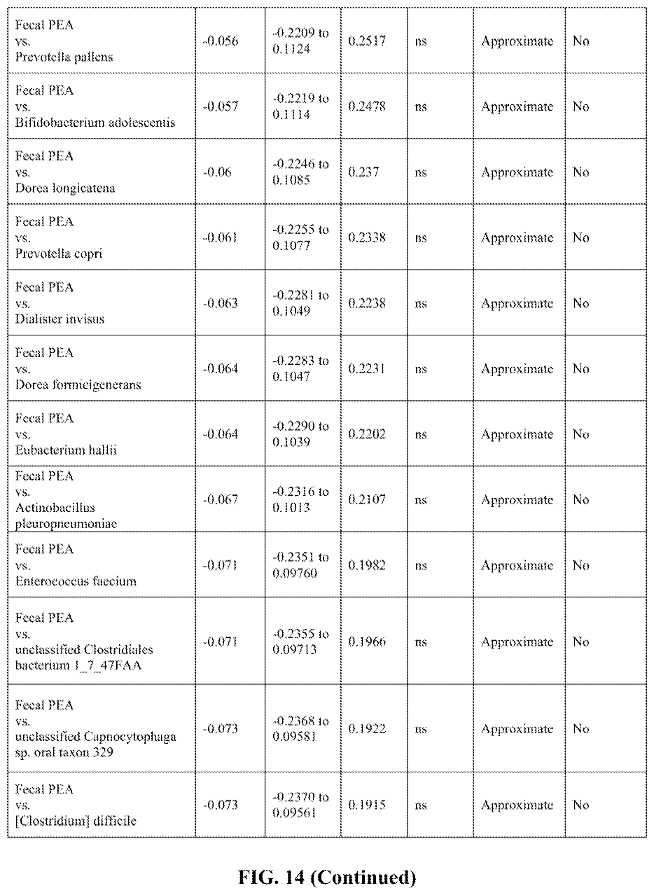

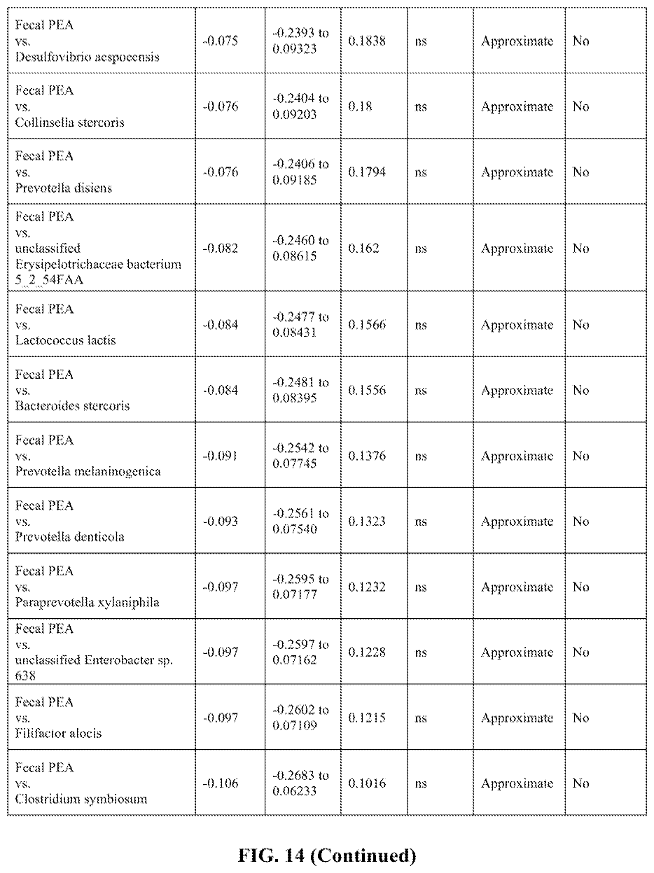

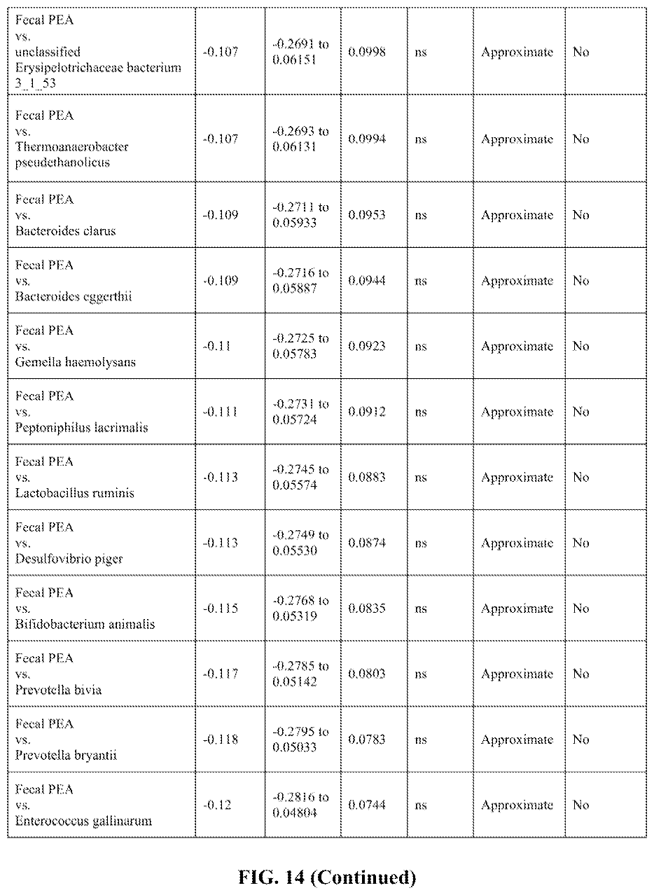

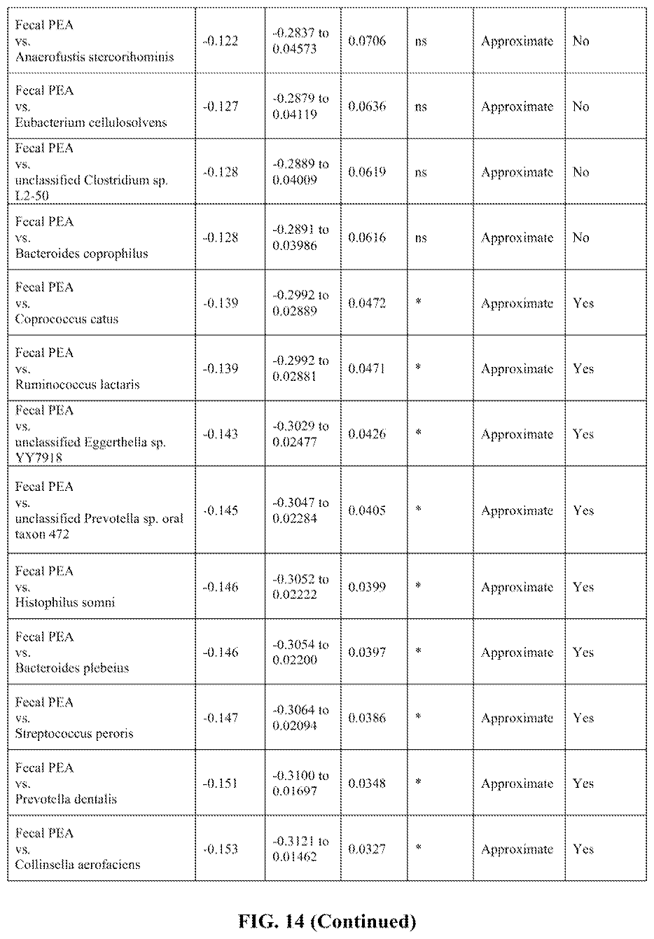

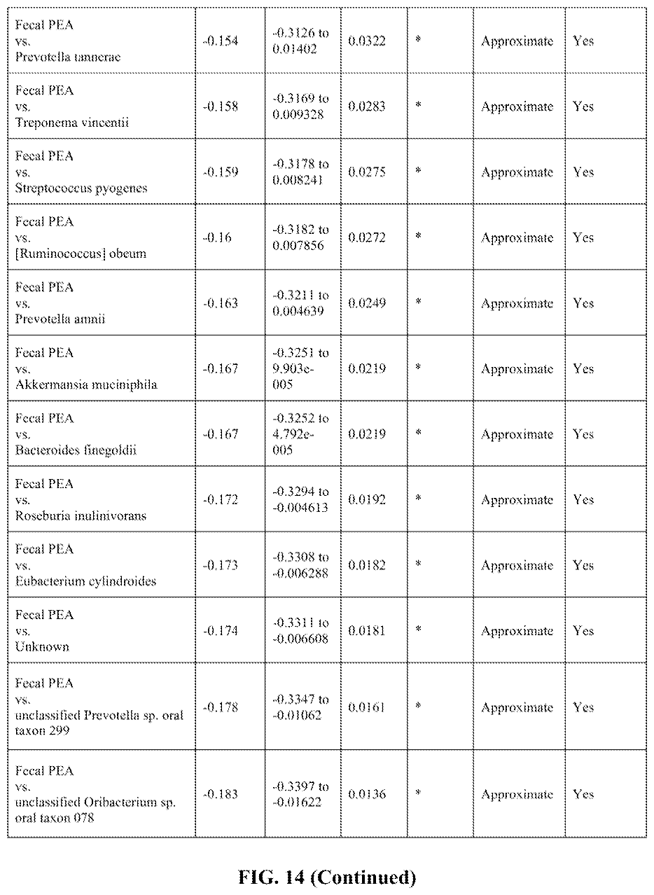

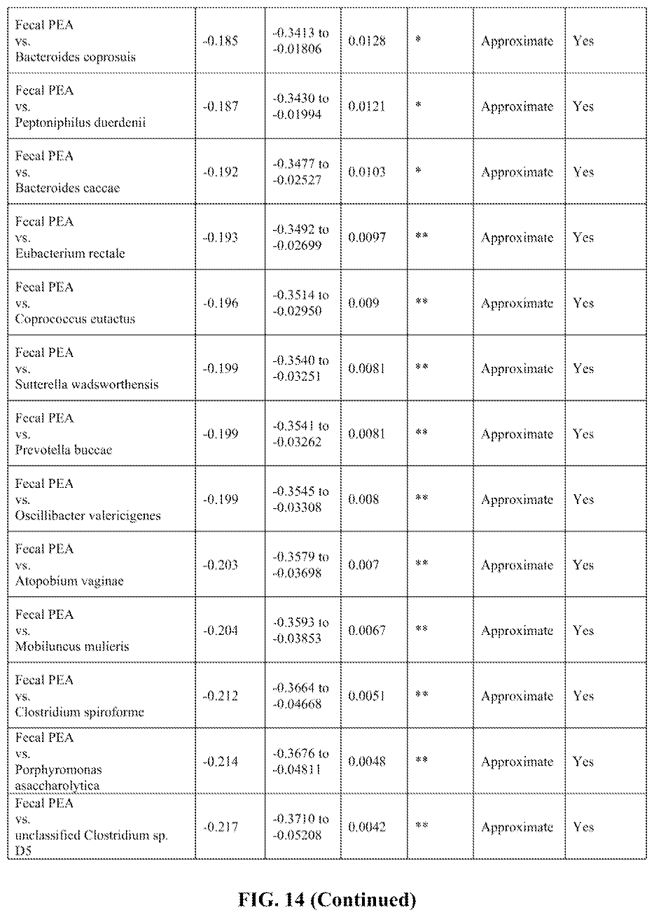

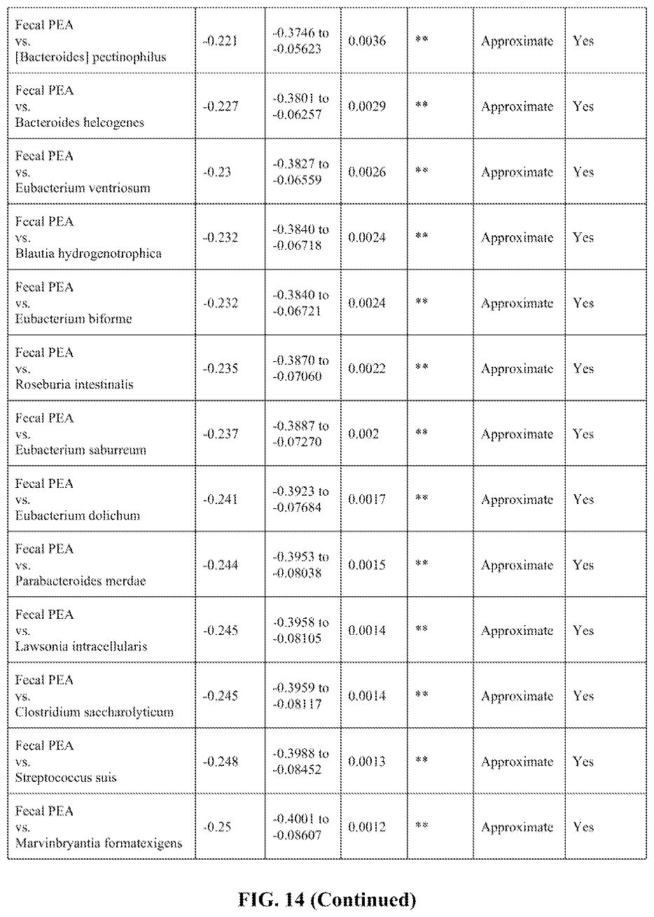

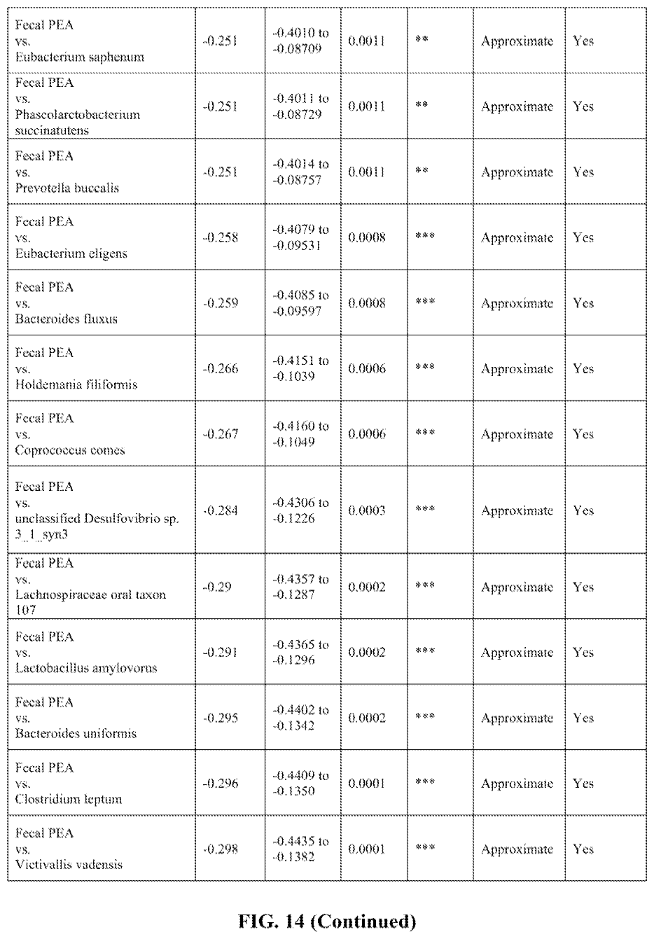

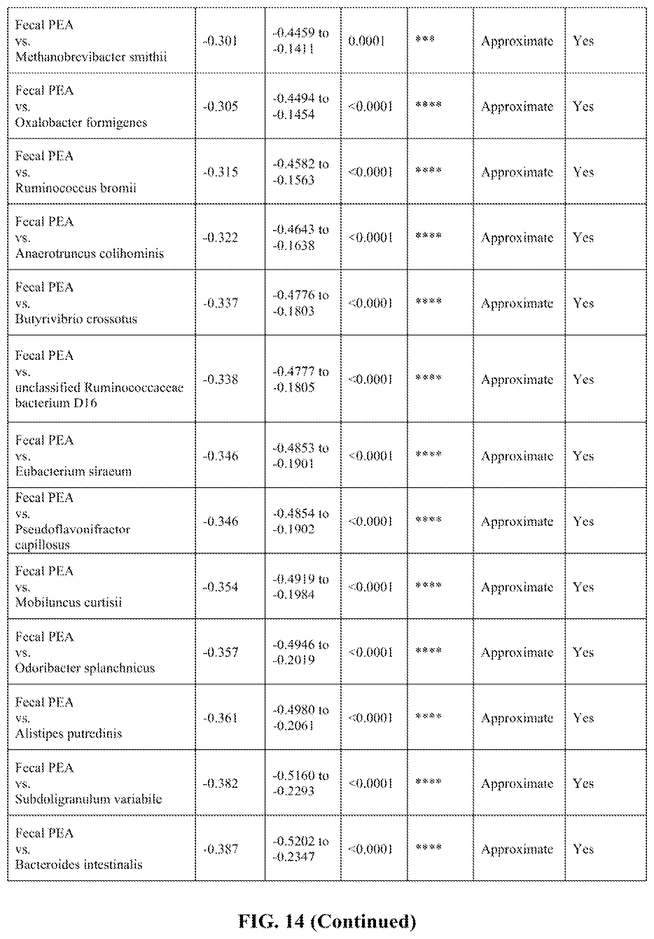

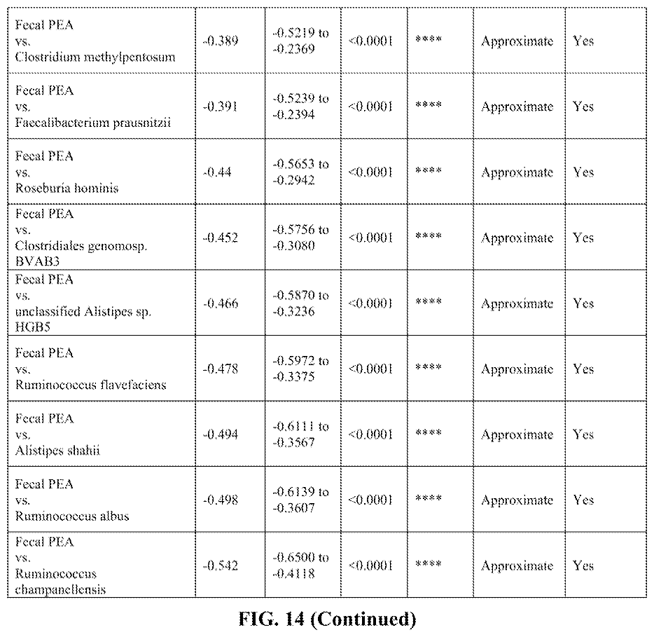

To evaluate the clinical relevance of the findings obtained from mouse studies, fecal phenethylamine, tryptamine, and tyramine levels were examined in an IBS-D patient cohort. It was found that fecal phenethylamine, tryptamine, and tyramine levels were significantly increased in patients with IBS-D (p<0.001 for phenethylamine and tryptamine, p=0.033 for tyramine, FIG. 3A-C), indicating that phenethylamine and tryptamine play a more important role compared with tyramine in mediating the pathogenic role of R. gnavus in IBS-D. The catalytic ability of gut microbiota from IBS-D and HC participants to convert aromatic amino acids into aromatic trace amines by batch culture studies using their fecal samples in vitro was then analyzed. Higher concentrations of phenethylamine and tryptamine, but not tyramine, were detected in the culture medium supplemented with the bacterial suspension of IBS-D fecal samples than in HC (p=0.039 and 0.018, FIG. 9A-C). Based on these results, the correlation between phenethylamine and tryptamine and the severity of diarrheal symptoms in patients with IBS-D was analyzed and it was found that phenethylamine and tryptamine levels were positively correlated with the severity of diarrheal symptoms as measured by the Bristol stool scale and serum 5-HT levels (r=0.2572/0.2366 and 0.2629/0.2418, p<0.001, FIG. 3D-G). Moreover, about 44% (128/290) and 51% (148/290) of the patients with IBS-D showed excessive phenethylamine and tryptamine levels in feces by using the 75% cut-off value as determined from the healthy controls (FIG. 3H-I). Correlation analysis also revealed that fecal phenethylamine and tryptamine levels were positively correlated with R. gnavus, with the highest r and p values among other gut bacterial species in patients with IBS-D (for R. gnavus, r=0.588 and 0.613, p<0.001, FIG. 3J-K and FIG. 14). Similarly, these findings were validated in another IBS-D cohort by showing that fecal phenethylamine and tryptamine were positively correlated with serum 5-HT levels, diarrheal symptoms, and R. gnavus abundance (p<0.05, for all cases, FIG. 9D-I).

### Phenethylamine/Tryptamine Accelerates Gastrointestinal Motility Through Increased Serotonin Biosynthesis

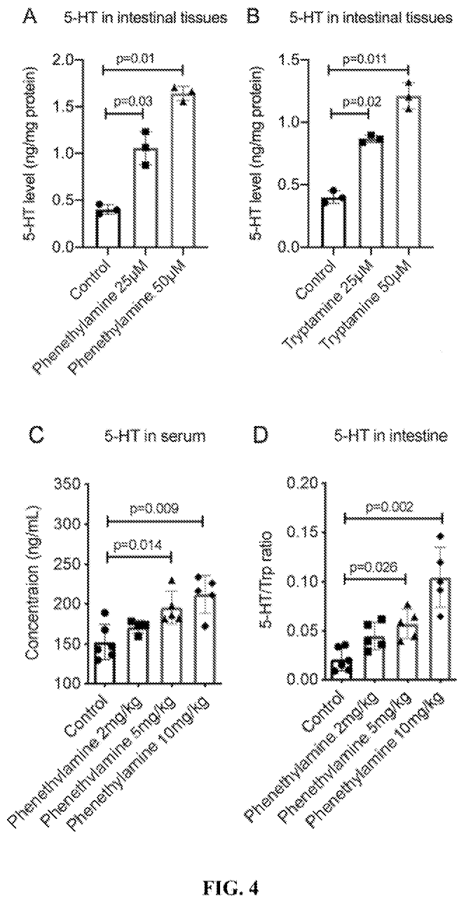

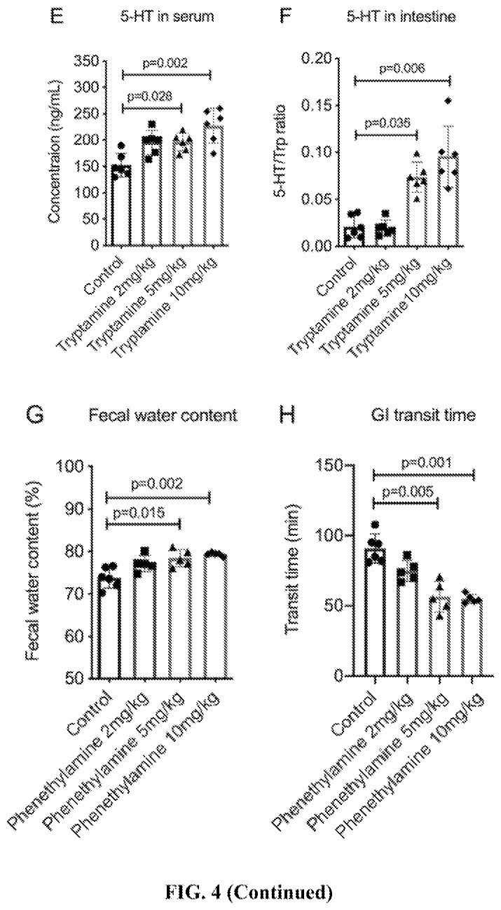

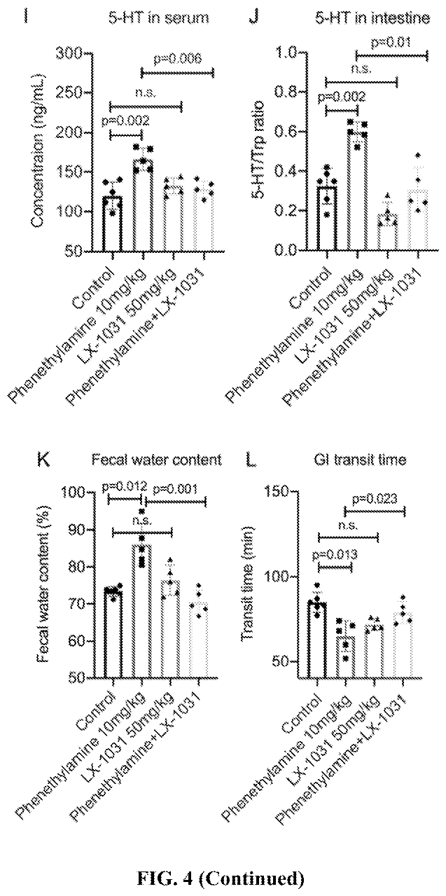

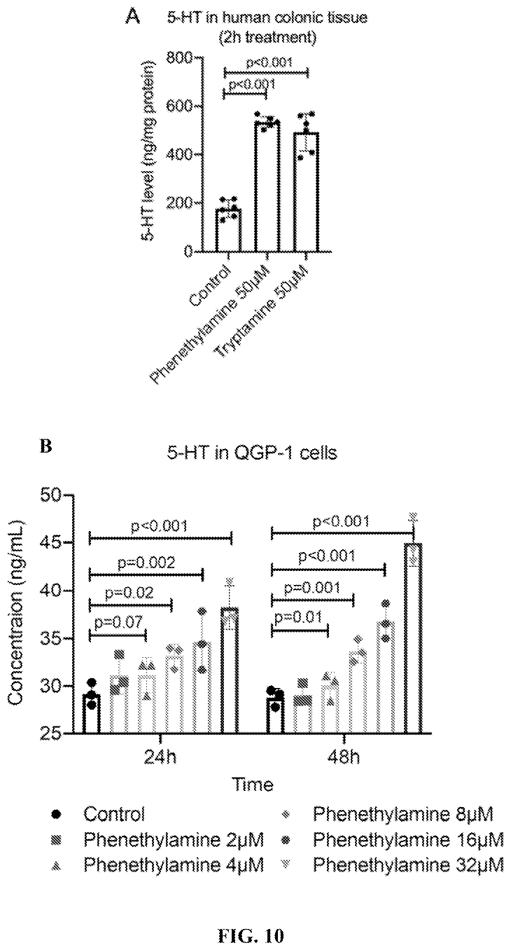

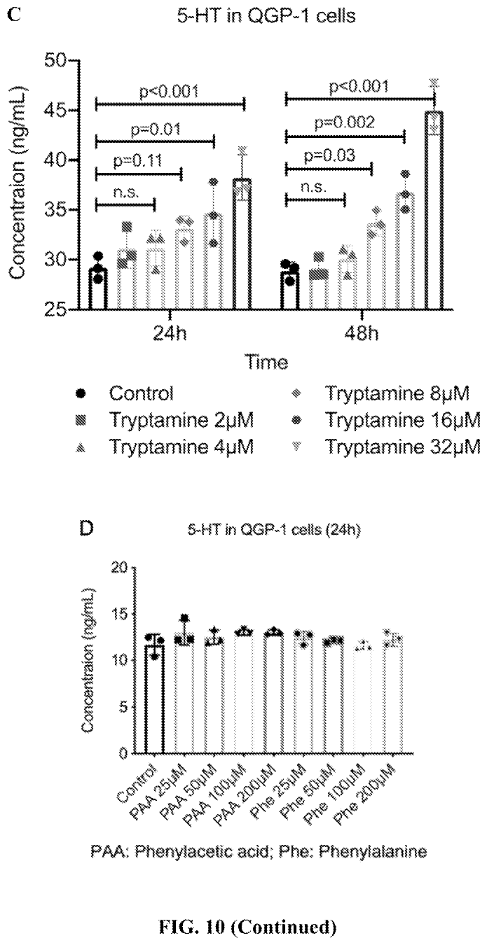

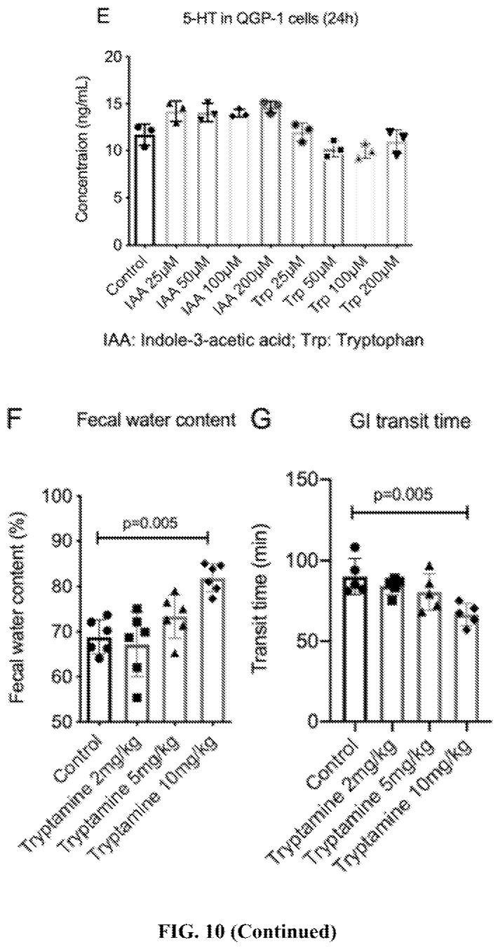

Since fecal phenethylamine and tryptamine levels were positively correlated with peripheral 5-HT and diarrhea-related symptoms, it was hypothesized that an increase in phenethylamine and tryptamine stimulates 5-HT production and, hence, upregulates GI transit and colonic secretion. First, it was showed that phenethylamine and tryptamine treatment significantly enhanced 5-HT production in mouse intestinal tissues and human colonic tissues cultured ex vivo for 4 h and 2 h (p<0.05, FIGS. 4A-B and 10A). Similar stimulatory effects of the phenethylamine and tryptamine treatment (24 and 48 h) on 5-HT production within the pathophysiological concentration range detected in patients with IBS-D were also observed in QGP-1 cells, a well-established human pancreatic endocrine cell line used for studying 5-HT production (FIG. 10B-C). In contrast, treatment with precursors and metabolites of phenethylamine and tryptamine, including phenylalanine (Phe), phenylacetic acid (PA), tryptophan (Trp), and indole acetic acid (IAA), did not change the 5-HT levels in QGP-1 cells (FIG. 10D-E), indicating that 5-HT stimulation in this in vitro EC cell model was specific to phenethylamine/tryptamine and not related metabolites.

Involvement of phenethylamine/tryptamine in the regulation of GI transit and colonic secretion in mice by modulating 5-HT production was then investigated. Treatment with phenethylamine or tryptamine within the pathophysiological concentrations by oral administration resulted in significantly elevated serum and intestinal 5-HT levels (p<0.01 in all cases, FIG. 4C-F and Table 1) along with accelerated GI transit and increased fecal water content in mice (p<0.01 in all cases; FIGS. 4G-H and 10F-G). Furthermore, the pharmacological blockade of 5-HT production by the TPH1 inhibitor LX-1031 effectively inhibited the phenethylamine-induced increase in 5-HT levels (p<0.01 in all cases, FIG. 4I-J) and significantly improved diarrhea-like symptoms in phenethylamine-treated mice (p<0.05, FIG. 4K-L). Collectively, these data indicate that R. gnavus overgrowth leads to increased fecal phenethylamine and tryptamine content, which promotes 5-HT biosynthesis in the gut and thereby leads to diarrheal symptoms observed in IBS-D.

TABLE 1
Phenethylamine level in intestinal tissues and luminal content

| Concentration (μg/g) | 0 min | 15 min | 30 min |
|---|---|---|---|
| Duodenum | 0.23 | 1082 | 879 |
| Ileum | 0.24 | 24 | 8 |
| Proximal colon | 12 | 116 | 38 |
| Luminal content in duodenum | 3 | 1245 | 1200 |
| Luminal content in ileum | 1.67 | 29.7 | 9.3 |
| Luminal content in proximal colon | 3.7 | 111.6 | 58.9 |

### Phenethylamine Stimulates Serotonin Production Via a TAAR1-Dependent Mechanism

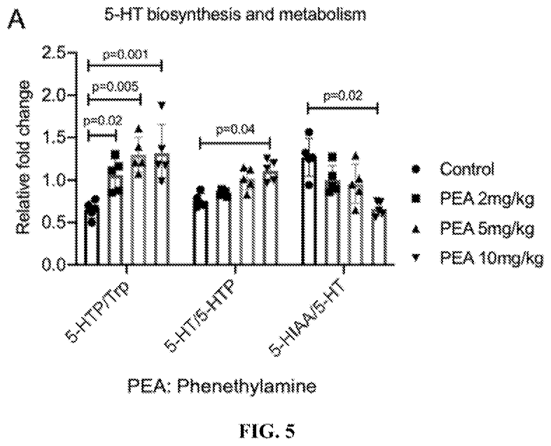

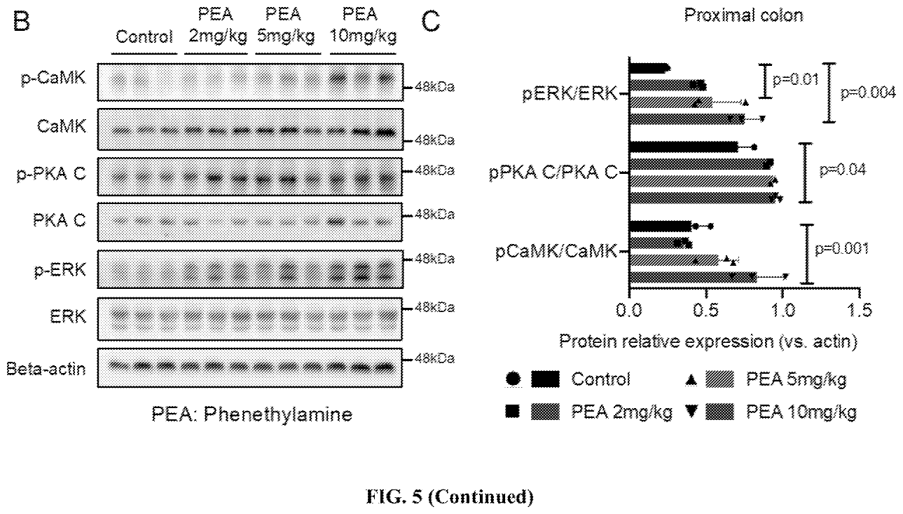

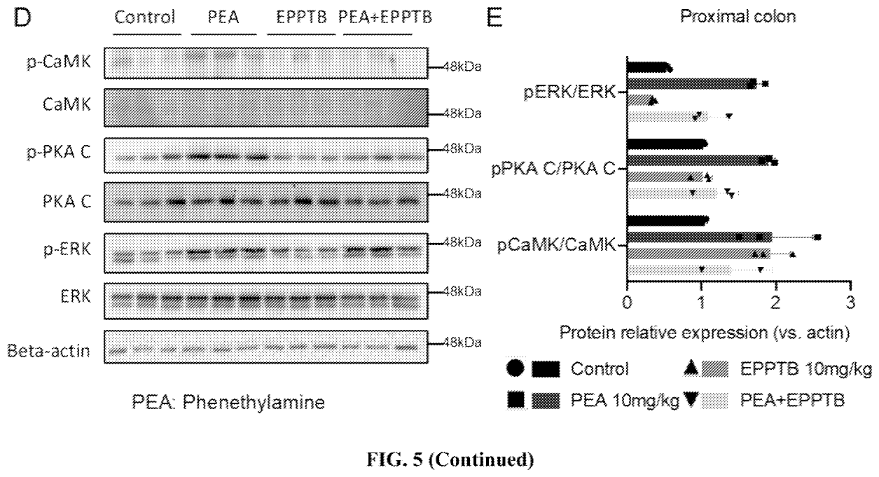

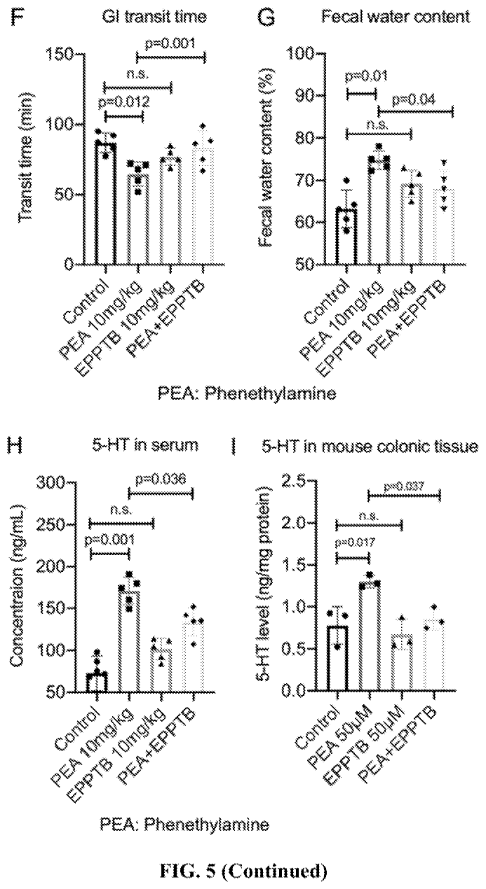

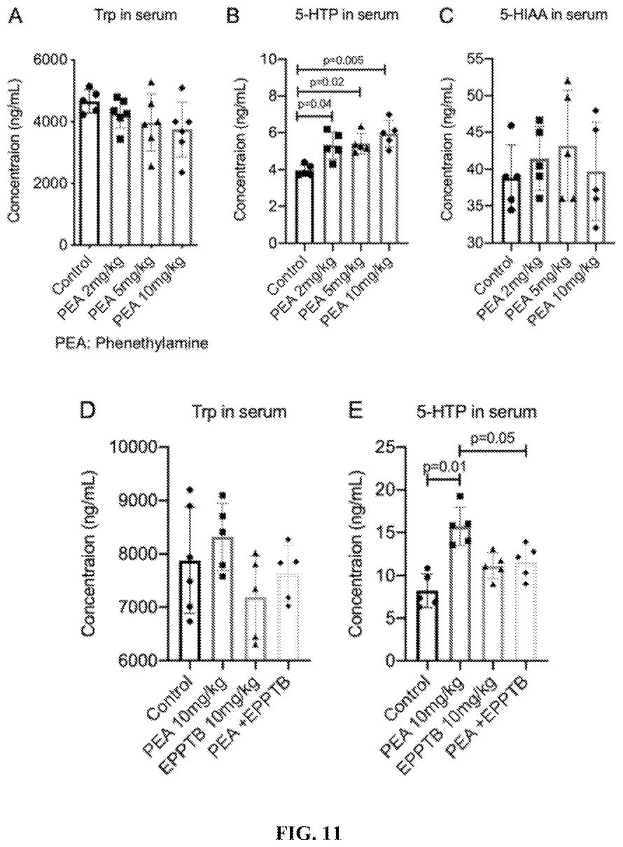

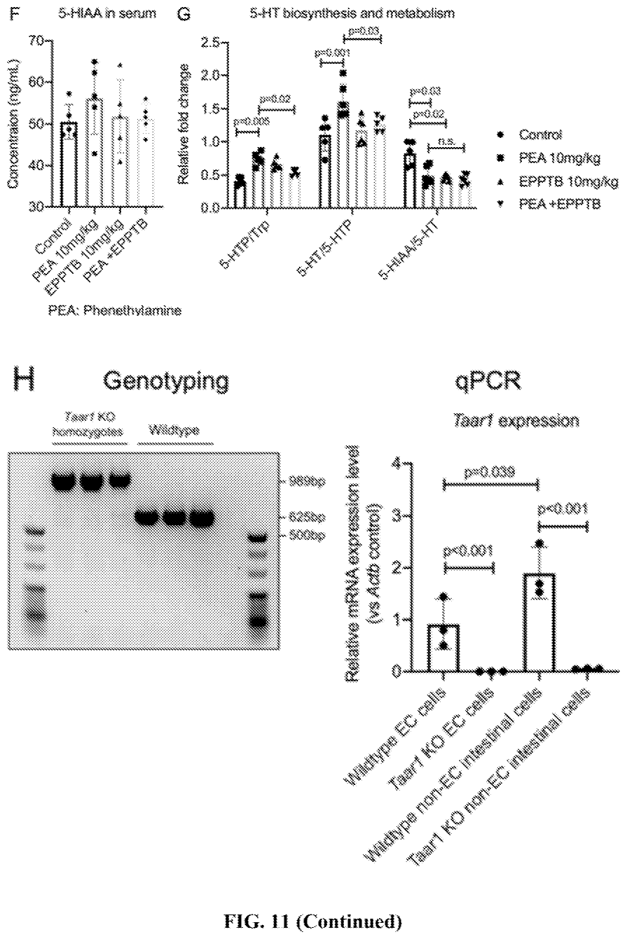

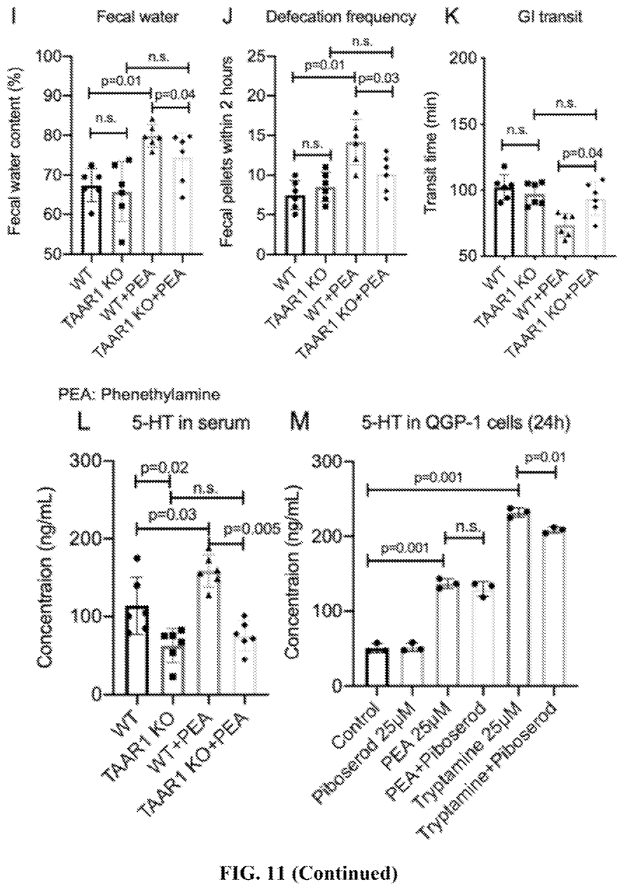

Peripheral 5-HT is synthesized from Trp through tryptophan hydroxylase 1 (TPH1, converting Trp to 5-HTP) and aromatic L-amino acid decarboxylase (AADC, converting 5-Hydroxytryptophan, or 5-HTP to 5-HT), which is subsequently metabolized into 5-hydroxyindole acetic acid (5-HIAA) by monoamine oxidase/aldehyde dehydrogenase (MAO/ALDH). To determine the mechanism underlying the stimulatory effects of phenethylamine on 5-HT production, the serum Trp, 5-HTP, and 5-HIAA levels, as well as the relative ratios of 5-HTP/Trp, 5-HT/5-HTP, and 5-HIAA/5-HT in phenethylamine-treated mice were analyzed. In addition to the upregulation of 5-HT, phenethylamine treatment led to an increase in circulating 5-HTP levels in mice in a dose-dependent manner (FIG. 11A-C). The results showed that 5-HTP/Trp and 5-HT/5-HTP ratios, but not the 5-HIAA/5-HT ratio, were increased in the serum of phenethylamine-treated mice (FIG. 5A), suggesting that the stimulatory effect of phenethylamine on 5-HT signaling is likely mediated through biosynthetic pathways (TPH1 and AADC; i.e., increased production).

Trace amine-associated receptor 1 (TAAR1), a G-protein coupled receptor for aromatic trace amines, including phenethylamine, tryptamine, and tyramine, is expressed in EC cells in the GI tract, as evidenced by the detection of TAAR1 expression in the pure culture of EC cells isolated from human ileum. Previous studies have revealed that the enzymes responsible for the biosynthesis of 5-HT, including TPH1 and AADC, are activated by downstream mediators of GPCR signaling, namely cyclic AMP-dependent protein kinase A (PKA) and calcium/calmodulin-dependent kinase (CaMKII). Indeed, it was found that phenethylamine activated PKA and CaMKIL, as confirmed by an increase in the phosphorylation of these proteins in the colonic tissues of mice treated with phenethylamine (p<0.05, FIG. 5B-C). Notably, the phenethylamine-induced activation of PKA and CaMKII was inhibited by the TAAR1-specific antagonist EPPTB (p<0.05, FIG. 5D-E). Blockade of TAAR1 activity by EPPTB also suppressed phenethylamine-induced GI transit acceleration, colonic secretion increase, and 5-HT production in mice (p<0.05, in all cases, FIG. 5F-H), as well as 5-HT elevation in mice intestine tissues cultured ex vivo (p<0.05, FIG. 5I). Similarly, increased levels of serum 5-HTP, as well as 5-HTP/Trp and 5-HT/5-HTP ratios induced by phenethylamine, were also inhibited by EPPTB treatment (p<0.05, FIG. 11D-G). Consistent with the results obtained from the pharmacological inhibition of phenethylamine, we demonstrated that genetic ablation of TAAR1 abolished phenethylamine-induced changes in 5-HT production, GI transit, and secretion in mice (p<0.05, in all cases; FIG. 11H-L), confirming that TAAR1 is a key receptor for phenethylamine in the context of GI function. These results suggest that phenethylamine-activated TAAR1 promotes GI transit and increases colonic secretion by stimulating 5-HT production.

### Phenethylamine/Tryptamine Produced by IBS-D-Associated Gut Microbiota Enhances Serotonin Synthesis and Gastrointestinal Transit In Vivo

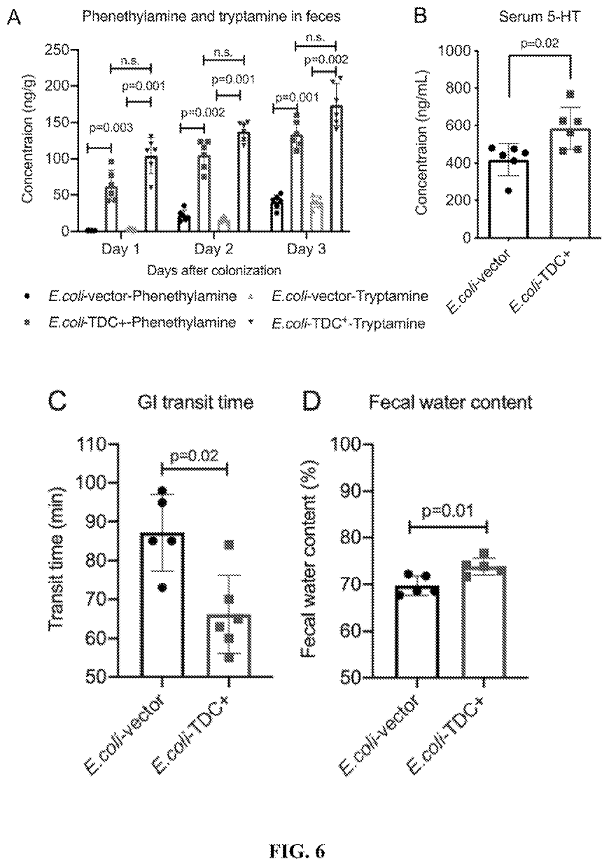

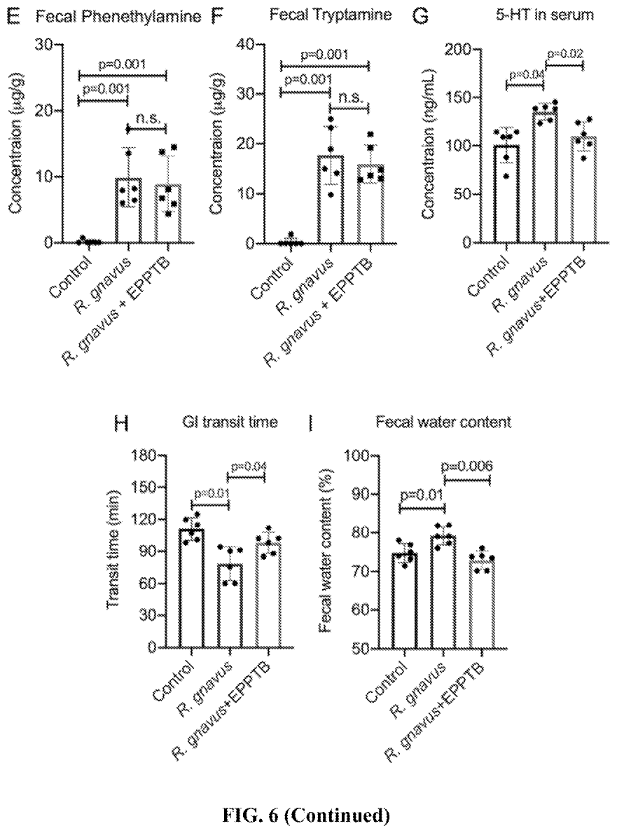

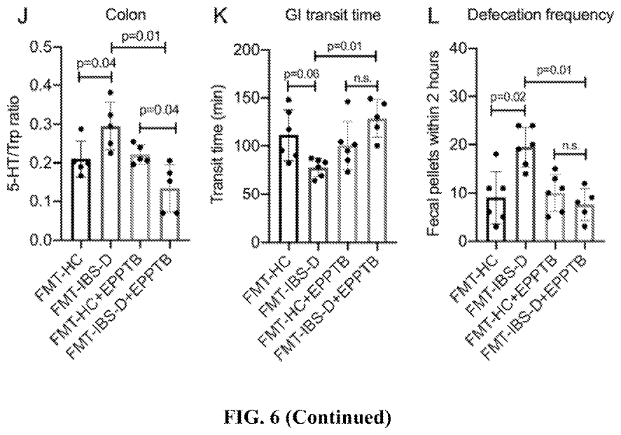

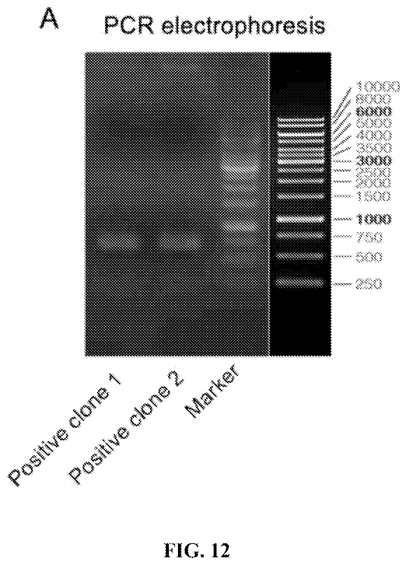

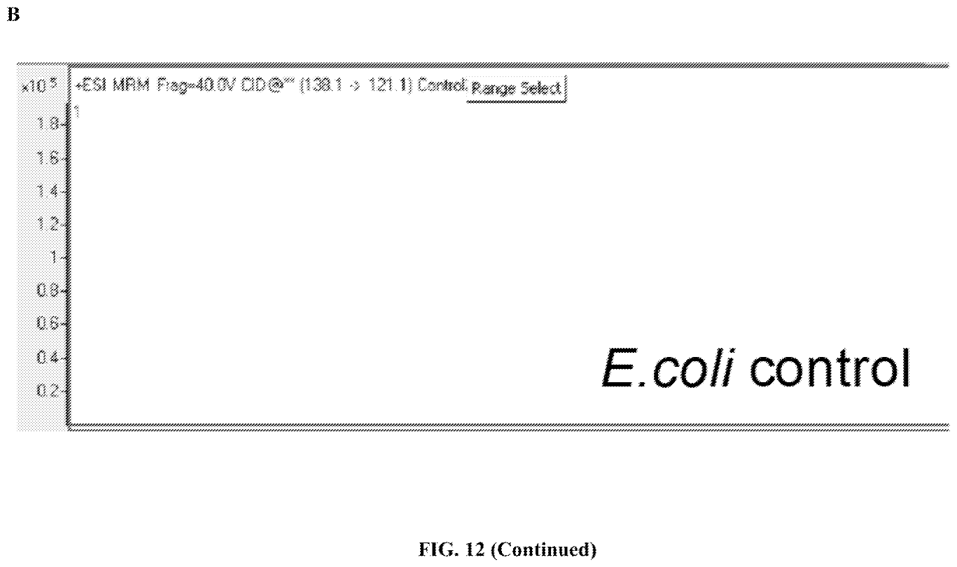

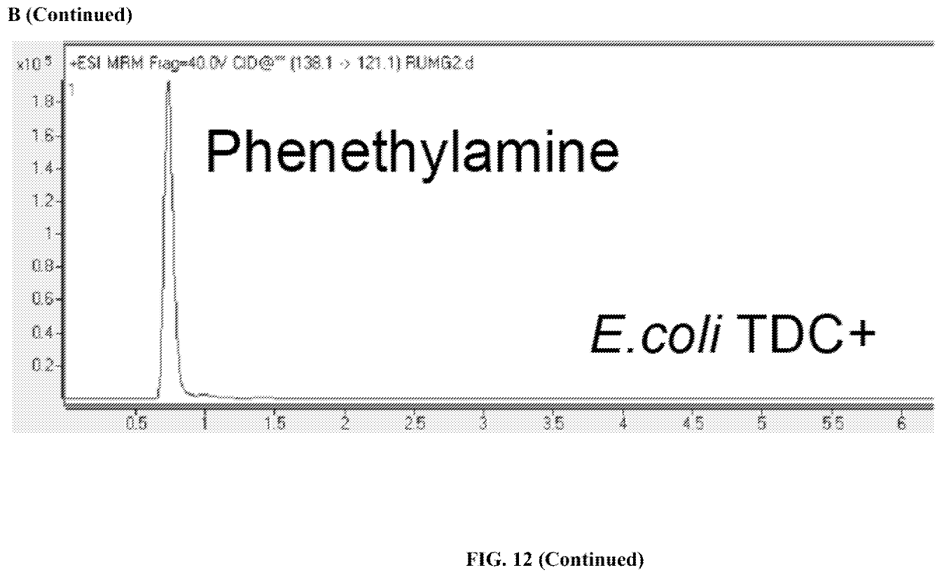

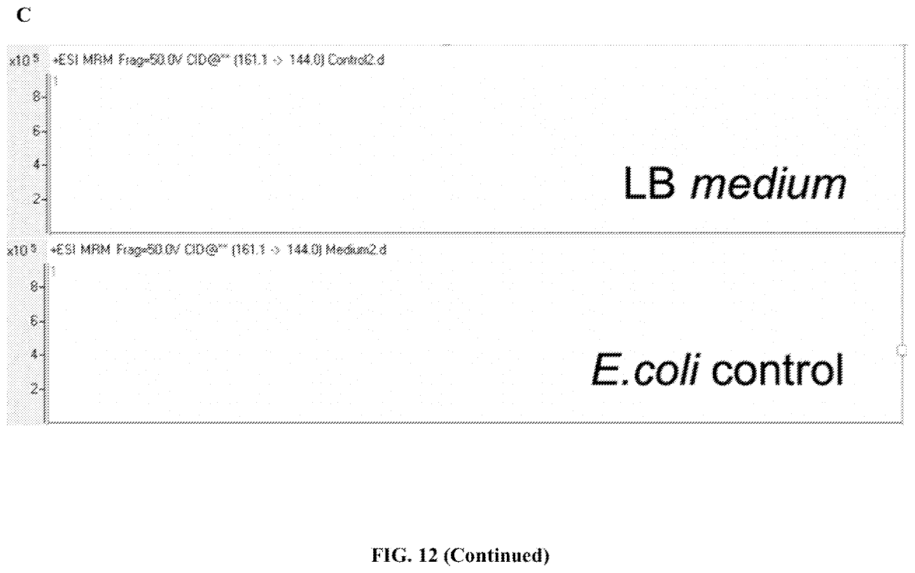

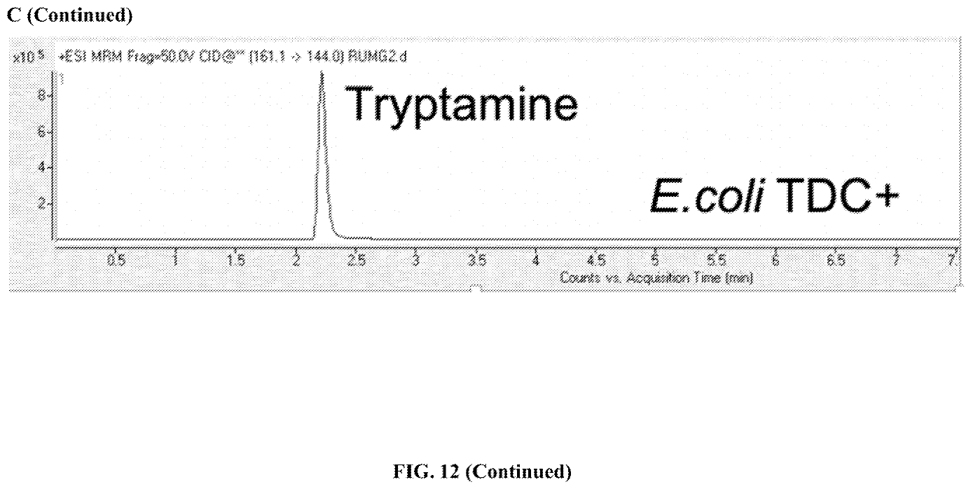

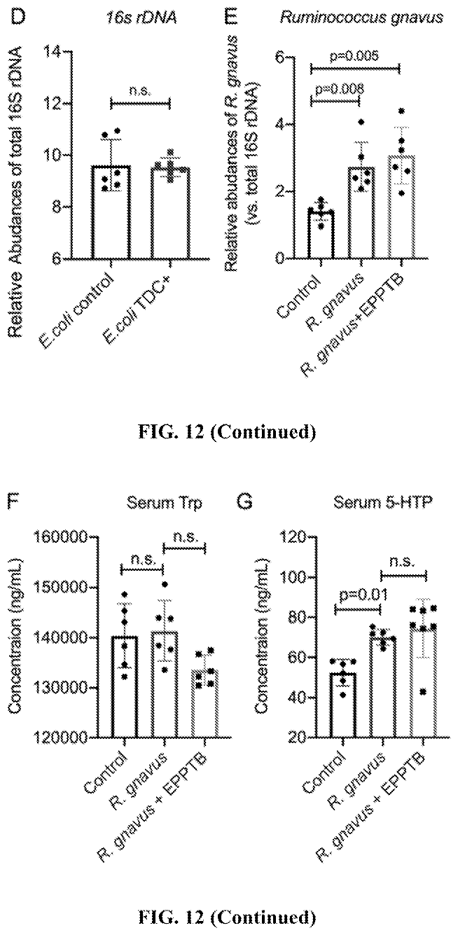

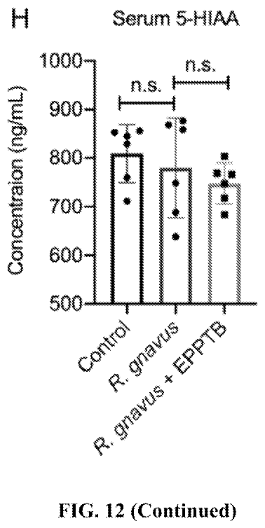

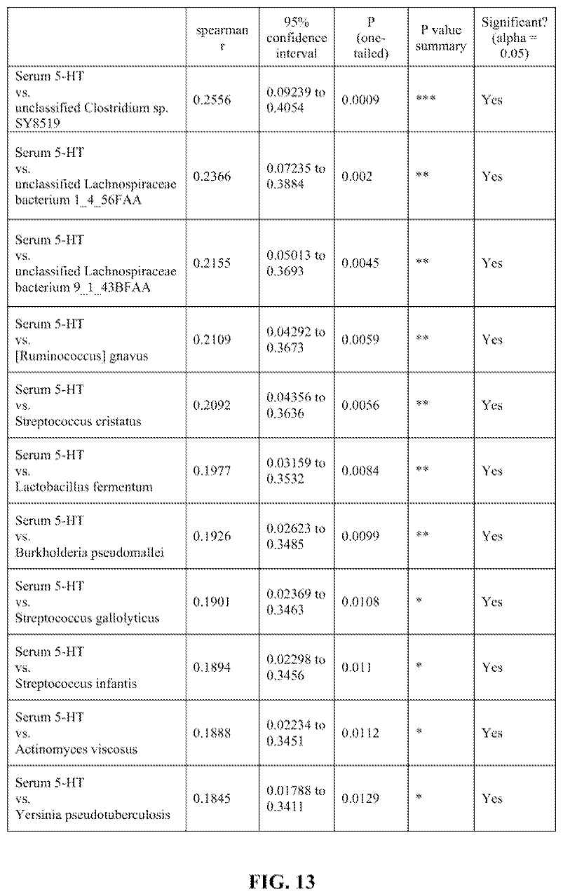

To study the in vivo action of phenethylamine/tryptamine on 5-HT production and GI motility, a plasmid expressing tryptophan decarboxylase sequence (TDC), an enzyme that catalyzes the conversion of Phe into phenethylamine and Trp into tryptamine from R. gnavus (strain ATCC 29149) was introduced into the Escherichia coli K12 strain. Antibiotic-treated mice were colonized by either vector-control E. coli or E. coli TDC⁺ by oral gavage. Successful integration of the plasmid was validated by PCR analyses, and the in vitro production of phenethylamine and tryptamine in the LB medium was assessed by LC-MS analysis (FIG. 12A-C). Antibiotic-treated mice colonized with E. coli TDC⁺ exhibited significantly elevated levels of phenethylamine and tryptamine in feces compared to mice colonized with the same doses of vector-control E. coli, confirming that the E. coli TDC⁺ strain produced phenethylamine and tryptamine in vivo (p<0.01 in all cases, FIGS. 6A and 12D). Consistently, increased serum 5-HT levels and fecal water content coupled with shortened GI transit time were observed in mice colonized with E. coli TDC⁺ compared with mice colonized with vector-control E. coli (p<0.05, FIG. 6B-D). These results demonstrated that bacteria engineered with TDC produced phenethylamine and tryptamine in vivo, which could enhance 5-HT production, leading to accelerated GI transit and increased colonic secretion.

To further confirm whether phenethylamine- and tryptamine-mediated TAAR1 signaling is involved in the elevation of 5-HT production and diarrhea-like symptoms induced by R. gnavus, the TAAR1 antagonist EPPTB was used to block the effects of phenethylamine and tryptamine on 5-HT production. Colonization of R. gnavus was shown to induce 5-HT elevation in serum as well as diarrhea-like symptoms, including accelerated GI transit and increased fecal water content, and these effects were suppressed by EPPTB treatment (p<0.05, FIGS. 6E-I and 12E-H). These results demonstrate that R. gnavus modulates 5-HT production and hence GI transit via phenethylamine/tryptamine-mediated TAAR1 signaling, suggesting that R. gnavus contributes to diarrhea symptoms in patients with IBS-D.

To investigate the therapeutic potential of targeting TAAR1 in the management of IBS-D, antibiotic-treated mice colonized with gut microbiota from patients with IBS-D or healthy controls were employed as a preclinical model of IBS-D. Antibiotic-treated mice colonized with IBS-D microbiota exhibited diarrhea-like symptoms characterized by increased GI transit and defecation frequency coupled with elevated 5HT biosynthesis and increased production of phenethylamine in the gut. Interestingly, the pathological changes induced by transplantation of IBS-D fecal microbiota were largely suppressed by the inhibition of TAAR1 activity with EPPTB (p<0.05, FIG. 6J-L and Table 2). These data highlight the therapeutic potential of targeting the phenethylamine/tryptamine-TAAR1 pathway for the management of IBS-D.

TABLE 2
Fecal phenethylamine and tryptamine level in selected
HC and IBS-D subjects for FMT experiment

| | No. | Phenethylamine (μg/g) | Tryptamine (μg/g) |
|---|---|---|---|
| IBS-D | 257 | 2.693544997 | 5.748852876 |
| IBS-D | 483 | 3.75615682 | 8.087285323 |
| IBS-D | 269 | 2.153258159 | 3.897287909 |
| IBS-D | 572 | 0.71648666 | 1.709083506 |
| IBS-D | 636 | 1.471128693 | 2.923746753 |
| IBS-D | 652 | 1.58983627 | 6.421256366 |
| IBS-D | 737 | 1.051451247 | 0.731837684 |
| IBS-D | 219 | 0.83430601 | 0.402565174 |
| Healthy | 1 | 0.044029087 | 0.004158084 |
| Healthy | 2 | 0.045170413 | 0.005314917 |
| Healthy | 3 | 0.162225184 | 0.503679858 |
| Healthy | 4 | 0.062869694 | 0.014673819 |
| Healthy | 5 | 0.049314673 | 0.003946205 |
| Healthy | 6 | 0.047132177 | 0.007279649 |
| Healthy | 7 | 0.051780426 | 0.030422599 |
| Healthy | 8 | 0.06401079 | 0.011414448 |

### Materials Availability

This study generated an E. coli K12 DH5 alpha bacteria strain that can produce phenethylamine and tryptamine.

### Experimental Models and Subject Details

### Human Study

To determine the changes in phenethylamine, tryptamine, tyramine, and 5-HT signaling in IBS-D patients, biological samples were collected in a human study previously described and approved by our institutional review board (HASC/15-16/0300 and HASC/16-17/0027) (ClinicalTrials.gov Identifier: NCT02822677). Briefly, patients with IBS (n=345) and healthy volunteers (n=91) were recruited according to inclusion criteria. Written consent was obtained from each participant prior to specimen collection. Biological samples, including serum, urine, and feces of all participants, were collected and transported to the laboratory in dry ice and stored at −80° C. Details of the clinical parameters and their correlations can be found in the Supplementary Information or provided upon request.

To validate the positive correlation among aromatic trace amines, serum 5-HT, R. gnavus abundance, and diarrhea symptoms in IBS-D patients, biological samples collected from another human study that was previously described were used (ClinicalTrials.gov Identifier: NCT03457324). This human study was approved by the Research Ethics Committee of Hong Kong Baptist University (HASC/16-17/C01) and the Clinical Research Ethics Committee of the Chinese University of Hong Kong (2016.718-T). Written consent was obtained from each participant prior to specimen collection. Briefly, biological samples, including serum and feces of patients with IBS-D (n=87), were used to validate previous findings. Details of the clinical parameters are provided upon request.

### Mouse Study

The mouse study was approved by the Committee on the Use of Human and Animal Subjects in Teaching and Research at Hong Kong Baptist University (Hong Kong SAR, China). All experiments were performed under the regulation of the animal (control of experiments) ordinance of the Department of Health, Hong Kong SAR, China. Male C57BL/6 mice aged 6-8 weeks and weighing 20-25 g were purchased from the Laboratory Animal Services Center, Chinese University of Hong Kong (Hong Kong SAR, China) and raised in the Animal Unit, School of Chinese Medicine, Hong Kong Baptist University. The mice were housed at a condition of 12 h light/dark cycle at a controlled temperature of approximately 25° C. with free access to food and water. In vivo experiments were performed following the ARRIVE guidelines.

### Antibiotic-Treated Mouse Model

A broad-spectrum antibiotic mixture (ABX) containing vancomycin (100 mg/kg), neomycin (200 mg/kg), metronidazole (200 mg/kg), and ampicillin (200 mg/kg) was used to establish an antibiotic-treated mouse model. Briefly, the antibiotic mixture was administered to mice by oral gavage for 10 consecutive days prior to fecal microbial transplantation (FMT) or the colonization study.

### Germ-Free Mouse Model

Germ-free mice were purchased from Nanjing GemPharmatech Co., and the monoassociation study was performed in sterile plastic isolators at the Laboratory Animal Facility of Nanjing GemPharmatech Co., as previously reported.

### Ex Vivo Colonic Tissues Study

Mouse and human colonic tissues were collected as previously described. To investigate the effects of phenethylamine on 5-HT levels in mouse and human colon tissues, the colonic tissues were cut into small pieces (0.5 cm) and placed in a 12-well cell culture plate. Colon pieces were added to DMEM/F12 medium containing 5% fetal bovine serum, penicillin-streptomycin (1×), and gentamycin (20 μg/ml), followed by treatment with phenethylamine and EPPTB, as indicated. Colon pieces were incubated at 37° C. with 5% CO2 and 95% air. A 200 μL volume of medium was collected from each well at the indicated time points for the analysis of 5-HT.

### QGP-1 Cells

QGP-1 cells were cultured in RPMI 1640 medium supplemented with 10% fetal bovine serum (FBS). QGP-1 is a human pancreatic endocrine cell line that produces 5-HT. To evaluate the effects of phenylalanine, phenethylamine, and phenylacetic acid on 5-HT production, QGP-1 cells were treated with or without phenylalanine, phenethylamine, and phenylacetic acid at the indicated concentrations and times.

### Enterochromaffin (EC) Cells Isolation

EC cells isolation was performed following a previous study using Chromogranin A in intestinal tissues of both wildtype and Taar1 KO mice. qPCR analysis was performed to examine the Taar1 expression in EC cells and non-EC intestinal cells. The Taar1 primers for qPCR analysis are [Forward: CTGTACAGTTTAATGGTGCTCATAATTCTGACC (SEQ ID NO:1) and Reverse: AGCATAGTAGCGGYCAATGGAGATGAAAGAC (SEQ ID NO:2)].

### Bacteria Strains

R. gnavus (strain ATCC 29149) was first grown on tryptic soy broth (TSB) agar plates and cultured in TSB broth using a single colony. R. gnavus (strain ATCC 29149) was collected from the medium by centrifuging at 3,500 rpm for 10 min at room temperature. These bacterial strains were then reconstituted in 200 μL of sterilized phosphate-buffered saline and administered to antibiotic-treated mice by oral gavage. Fecal samples were collected daily to measure fecal phenethylamine levels in antibiotic-treated mice before and after oral administration of bacterial strains.

Phenethylamine- and tryptamine-producing E. coli K12 were constructed using the tryptophan decarboxylase (TDC) gene (A7B1V0) from R. gnavus. The TDC gene was cloned into the vector and the resulting plasmid was transferred into E. coli K12 as previously described. The insertion of the TDC gene into E. coli K12 was confirmed by PCR and by in vitro phenethylamine and tryptamine production in LB broth containing 0.25% phenylalanine. A vector-only control strain, E. coli K12, was also constructed. These bacterial strains were also collected as previously described, prepared in 200 μL of sterilized PBS, and then delivered to antibiotic-treated mice by oral gavage.

### Methods Details

### Metabolites Quantification

An Agilent 1290 Infinity II UPLC system coupled to a triple quadrupole (QQQ) 6470 mass spectrometer was used for targeted metabolomics profiling. A Waters BEH 2.1×50 mm C18 1.7 μm column with a pre-column was used. The mobile phase used in LC-MS-QQQ was solution A (water with 0.1% formic acid) and solution B (acetonitrile with 0.1% formic acid). The gradients were set as 2% B (0-0.5 min), 2%-30% B (0.5-4 min), 30/6-100% B (4-6 min), 100% B (6-8 min), 100%-2% B (8-8.1 min) and maintain in 2% B (8.1-10 min). The MS data were collected and processed using Agilent software. The standard list, MRM transition, fragmentor, and collision energy are listed in (Table 3).

TABLE 3
MRM transition and parameters used for metabolites
quantification, Related to STAR Methods

| | | Prec | Prod | Frag | CE | |
|---|---|---|---|---|---|---|
| No | Compound Name | Ion | Ion | (V) | (V) | Polarity |
| 1 | L-tryptophan (Trp) | 205.1 | 188 | 90 | 5 | Positive |
| | L-tryptophan (Trp) | 205.1 | 118 | 90 | 29 | Positive |
| 2 | L-hydroxy-Tryptophan | 221.1 | 204.1 | 50 | 5 | Positive |
| | (5-HTP) | | | | | |
| | L-hydroxy- Tryptophan | 221.1. | 162.1 | 50 | 13 | Positive |
| | (5-HTP) | | | | | |
| 3 | Serotonin(5-HT) | 177.1 | 160 | 90 | 9 | Positive |
| | Serotonin (5-HT) | 177.1 | 115 | 90 | 29 | Positive |
| 4 | 5-Hydroxyindoleacetic | 192.1 | 146 | 110 | 13 | Positive |
| | acid (5-HIAA) | | | | | |
| | 5-Hydroxyindoleacetic | 192.1 | 91.1 | 110 | 37 | Positive |
| | acid (5-HIAA) | | | | | |
| 5 | Phenylalanine (Phe) | 166.1 | 120.1 | 102 | 9 | Positive |
| | Phenylalanine (Phe) | 166.1 | 103.1 | 102 | 29 | Positive |
| 6 | Phenethylamine | 122.1 | 103.1 | 40 | 21 | Positive |
| | Phenethylamine | 122.1 | 77.1 | 40 | 29 | Positive |
| 7 | Tryptophan-d5 | 210.2 | 192.2 | 90 | 15 | Positive |
| | Tryptophan-d5 | 210.2 | 150 | 90 | 25 | Positive |
| 8 | Tyramine | 138.1 | 121.1 | 50 | 37 | Positive |
| | Tyramine | 138.1 | 77.1 | 40 | 9 | Positive |
| 9 | Tryptamine | 161.1 | 144 | 50 | 9 | Positive |
| | Tryptamine | 161.1 | 115.1 | 50 | 37 | Positive |
| 10 | Tyrosine | 182.1 | 136.1 | 102 | 9 | Positive |
| | Tyrosine | 182.1 | 91.1. | 102 | 25 | Positive |

### Batch Culture of Fecal Samples

Approximately 50 mg of fecal samples was mixed with 20× sterilized 1×PBS (m/v) and homogenized with a TissueLyzer after adding steel beads. 20 μL fecal suspension from each sample was inoculated in 2 mL TSB supplemented with 0.25% Phe and incubated overnight under anaerobic conditions at 37° C. After incubation, 100 μL of the medium was used to determine the Phe and phenethylamine levels. Briefly, 400 μL MeOH was added to 100 μL medium and vigorously vortexed. After that, the mixture was centrifuged at 15,000 rpm for 10 min at 4° C. The supernatant (200 μL) was used for LC-MS analysis.

### Phenethylamine and Tryptamine Administration

To study phenethylamine and tryptamine effects on 5-HT production, phenethylamine and tryptamine at a dosage of 2 mg/kg, 5 mg/kg and 10 mg/kg (dissolved in 0.5% CMC-Na, sodium carboxymethyl cellulose) were administered to mice by oral gavage. Mice treated with/without phenethylamine and tryptamine were sacrificed under isoflurane anesthesia. Biological samples including serum, duodenum, ileum, proximal colon and distal colon tissues and luminal contents in each intestinal segments were collected at indicated dosages and times and stored at −80° C. until analysis.

### In Vivo Measurements

For the fecal pellet water content test, 1.5 mL Eppendorf tubes were pre-weighed and used to collect fecal pellets from mice immediately after defecation. The tubes were then closed to measure wet weight. Afterward, the tubes were opened and placed in an oven at 60° C. overnight to measure the dry weight. Fecal pellet water content was measured by subtracting the dry weight from the wet weight and normalizing it to the wet pellet mass. Fecal pellets from mice were also counted within 2 h of the defecation frequency test.

Carmine red assay: Mice were administered 300 μL of 6% carmine red solution (prepared by 0.5% methylcellulose) by oral gavage to measure the GI transit time after treatment with phenethylamine at the indicated dosages. The mice were placed on white sheet paper to track the red pellet in their cages after oral gavage. The total time taken for the appearance of the first red pellet after oral gavage was recorded as gut transit time.

### Western Blotting Analysis

Colonic tissues were lysed in RIPA buffer containing protease inhibitor cocktails in a tissuelyzer. The tissue lysates were centrifuged at 15,000 rpm for 15 min at 4° C. and quantified by BCA kit for protein concentration. The protein content normalized supernatant was mixed with 5× loading buffer and heated at 98° C. in a dry bath for 10 min. The samples were evaluated according to the western blotting protocol (Abcam). The blots were incubated with HRP-conjugated anti-rabbit IgG or anti-mouse IgG and reacted with enhanced chemiluminescence.

### Transplantation of Human Microbiota (FMT) in Antibiotic-Treated Mice

Fecal samples from IBS-D patients and HC participants (n=8/group) were prepared as suspensions in PBS at a concentration of (100 mg/mL). Antibiotic-treated mice were used as recipients of HC and IBS-D microbiota and were orally gavaged daily with a human microbiota suspension at a dose of 500 mg/kg for 5 consecutive days. After the FMT, GI transit, stool consistency, and defecation frequency were measured. Fecal samples were collected prior to FMT (after ABX intervention) and after FMT.

### Phenethylamine and Tryptamine-Producing Bacteria Analysis in IBS-D Patients

Metagenomic data of patients with IBS-D and healthy volunteers were obtained as previously described³. Correlation analysis was conducted between serum 5-HT levels and fecal phenethylamine levels with phenethylamine- and tryptamine-producing bacteria derived from patients with IBS-D.

16S rRNA Analysis

Fecal samples of antibiotic-treated mice after colonization of either engineered E. coli and R. gnavus were processed using QIAGEN Genomic DNA Extraction kit to isolate DNA according to the manufacturer's instructions. Fecal DNA (50 ng/μL) of each sample was used for determining the abundances of total bacteria and R. gnavus using related primers.

### Generation of Taar1 Gene Knockout Mice

Taar1 (NM_053205) knockout mouse model (C57Bl/6J) was generated by CRISPR/Cas-mediated genome engineering. All the Taar1 KO mice and their wildtype littermates were genotyped using PCR and Southern blot [F1: GACAAAACGTAGTTGGAAGACTGA (SEQ ID NO:3), R1: GTGTGCCTAGAAACCTTAACATCTG (SEQ ID NO:4), R2: AATGTTTGTGATAGCGTGGCAAAG (SEQ ID NO:5)]. The product size of F1 and R1 is 989 bp while the product size of F1 and R2 is 625 bp. Southern blot of Taar1 homozygotes mice has only one band with 989 bp, while blots of wildtype littermates have only one band with 625 bp and heterozygotes mice have two bands with 989 bp and 625 bp.

### Statistical Analysis

All the results were obtained from multiple experiments (at least three independent experiments). Data were expressed as average with SD or SEM values where appropriate. Significance p-values were calculated using GraphPad Prism 8, and p-values less than 0.05 was regarded as statistically significant. Spearman's rank coefficient correlation analysis was used for metabolite quantity correlation and the correlation between serum 5-HT level, fecal phenethylamine level, and phenethylamine-producing bacteria derived from patients with IBS-D. The Wilcoxon rank-sum two-tailed test was used to determine metabolite differences between IBS-D and healthy participants. The Wilcoxon one-tailed test was used to determine whether IBS-D-associated gut microbiota produces higher levels of phenethylamine in the metagenomic data. Unpaired Student's t-tests or one-way ANOVA were used in other experiments, as indicated.

The Positive Association Between Ruminococcus gnavus, Peripheral Serotonin and Severity of Diarrhea Symptoms in IBS-D

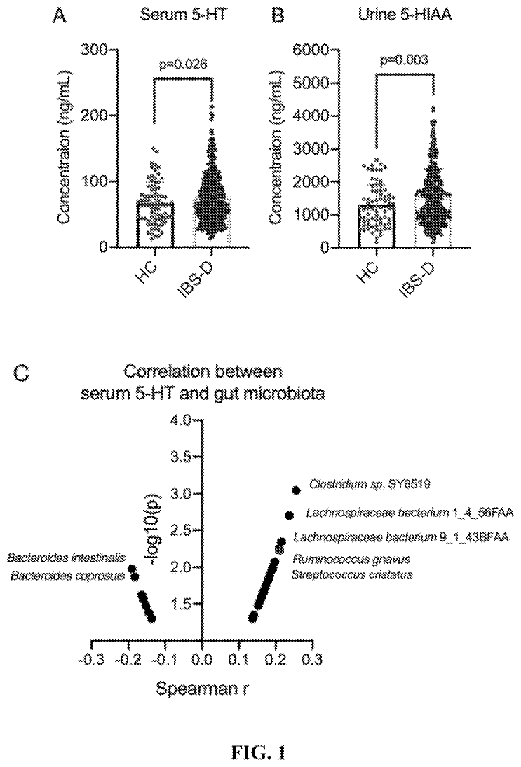

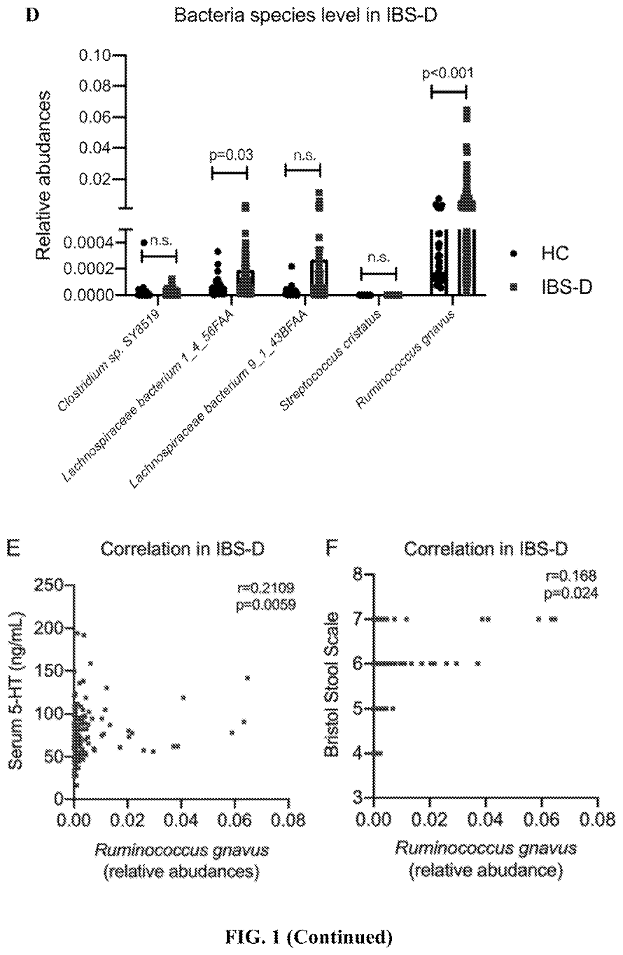

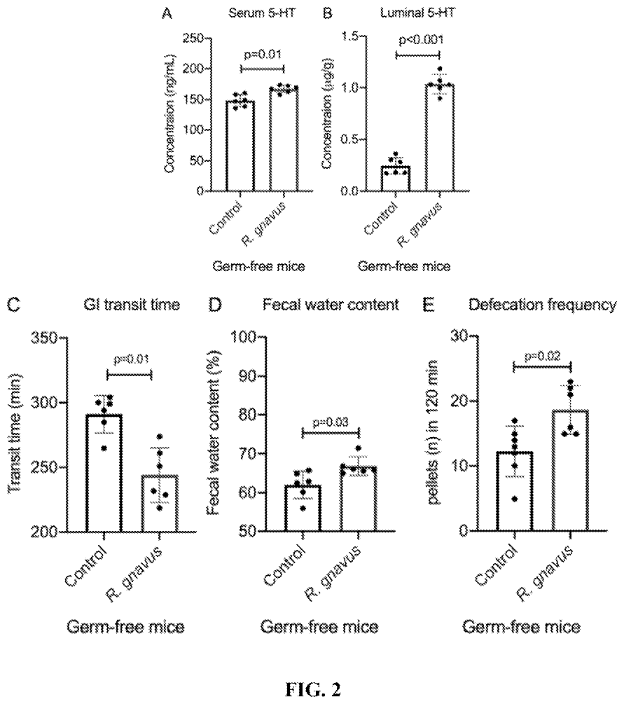

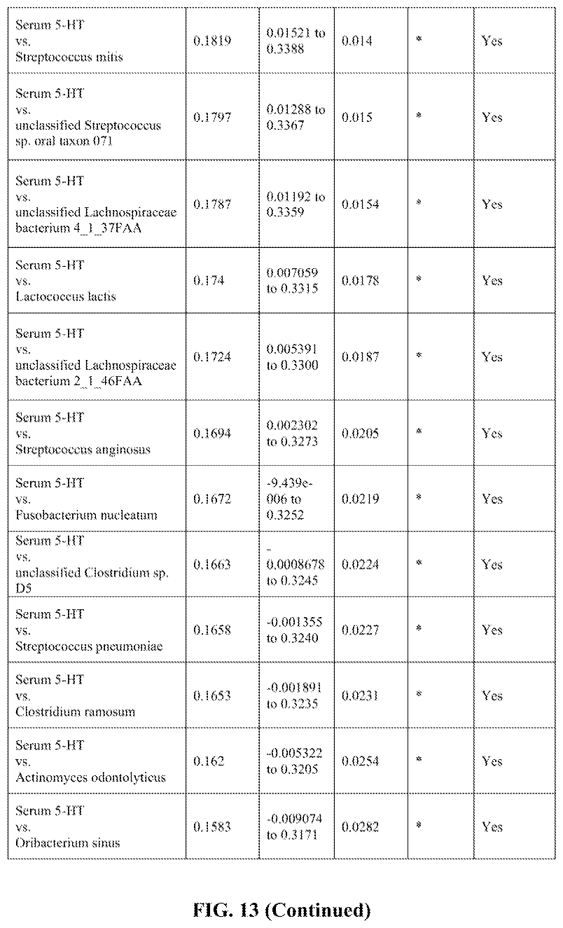

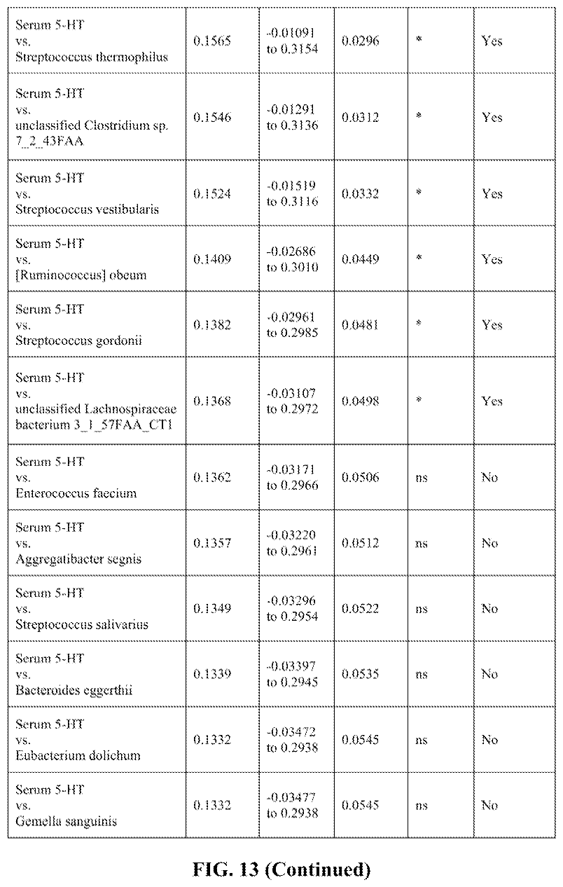

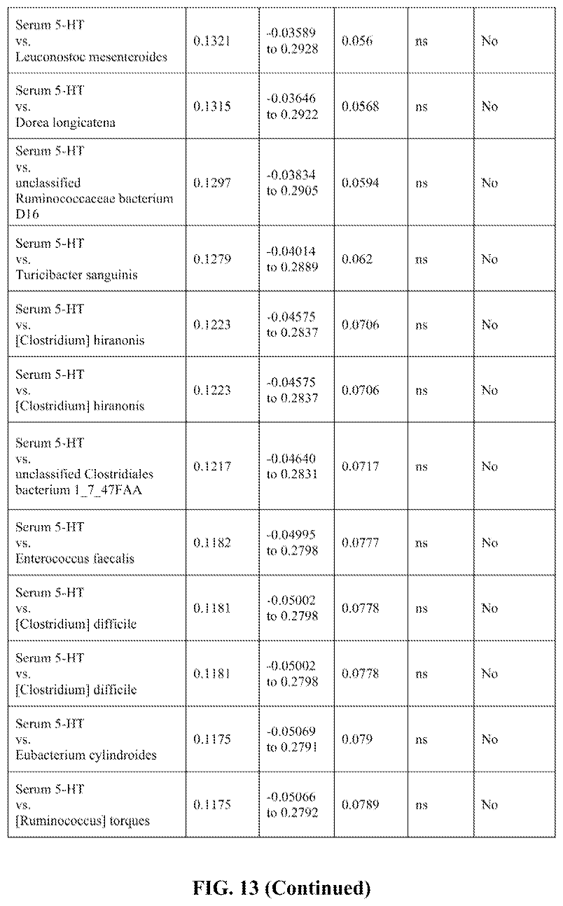

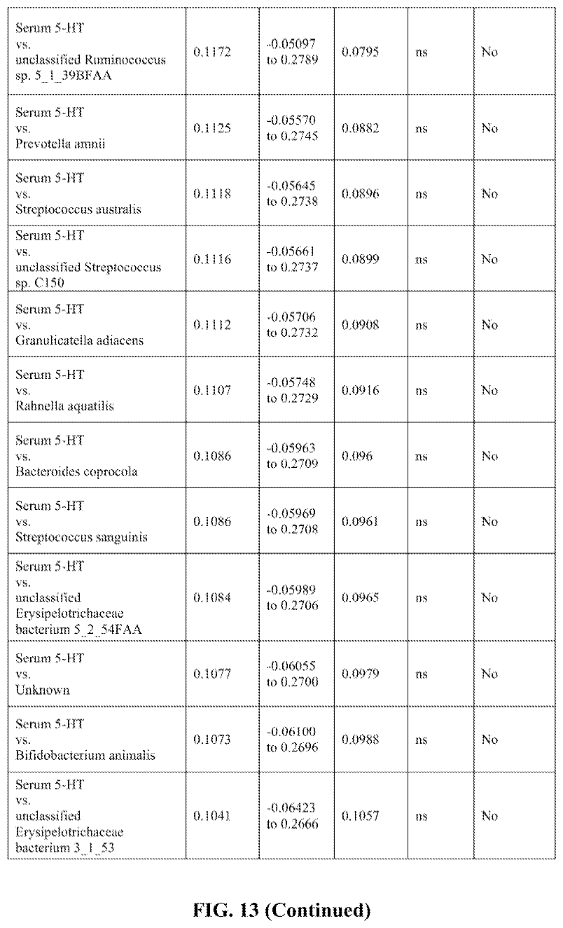

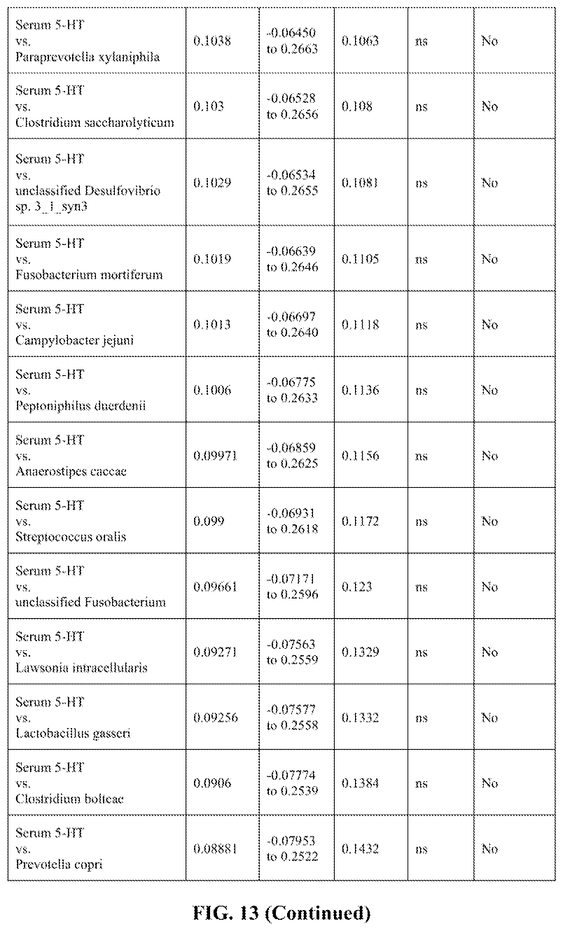

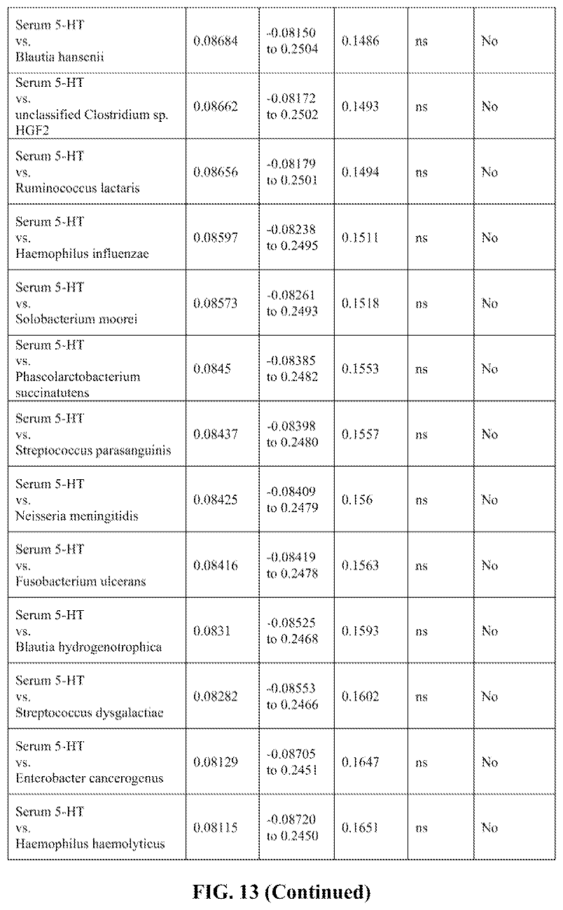

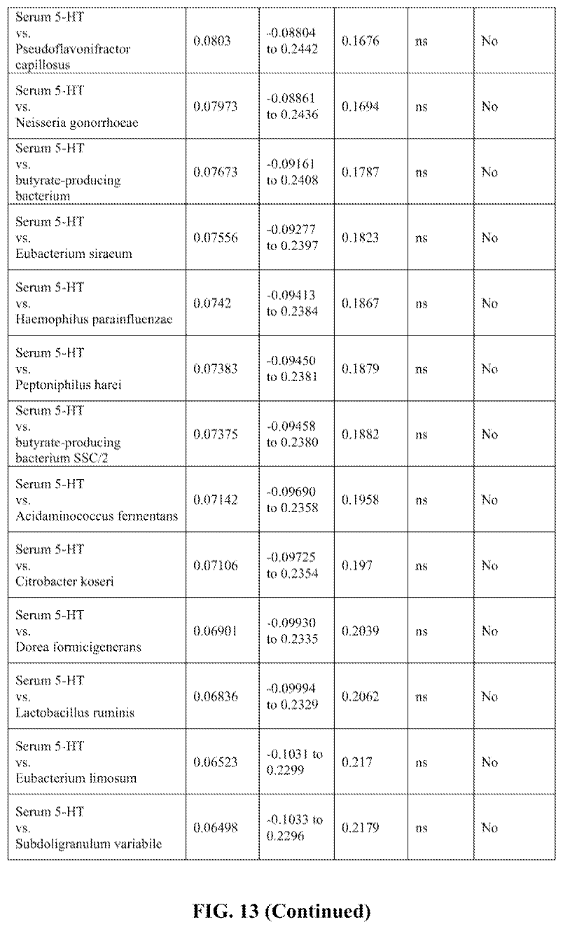

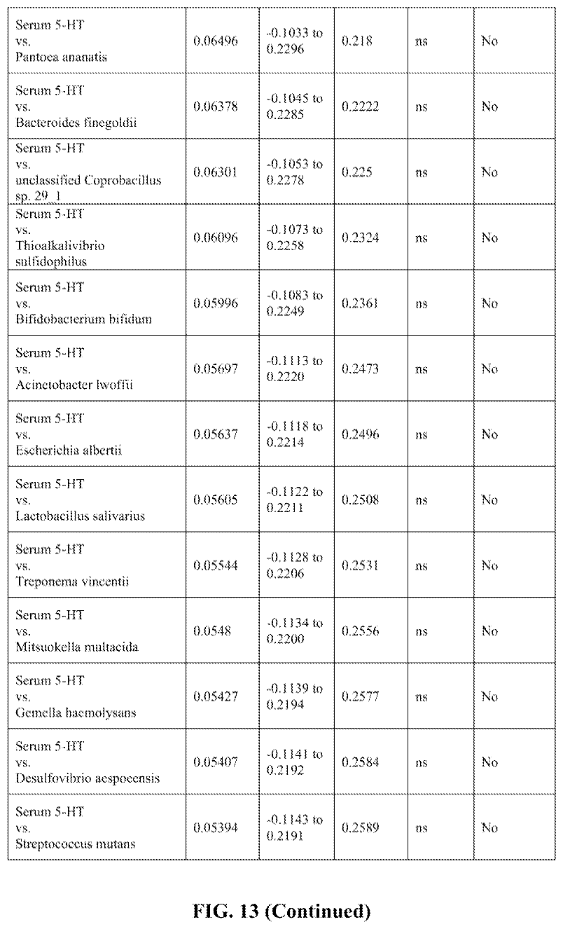

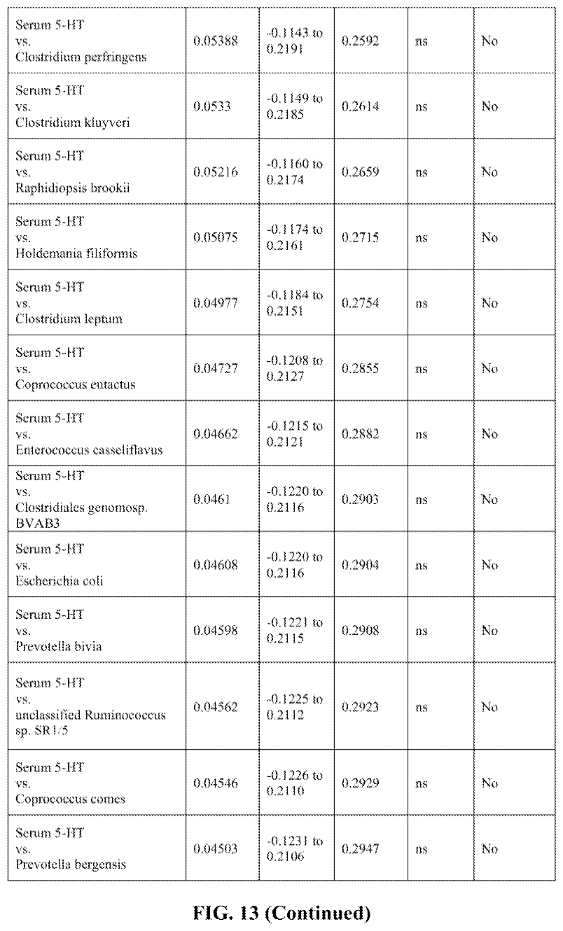

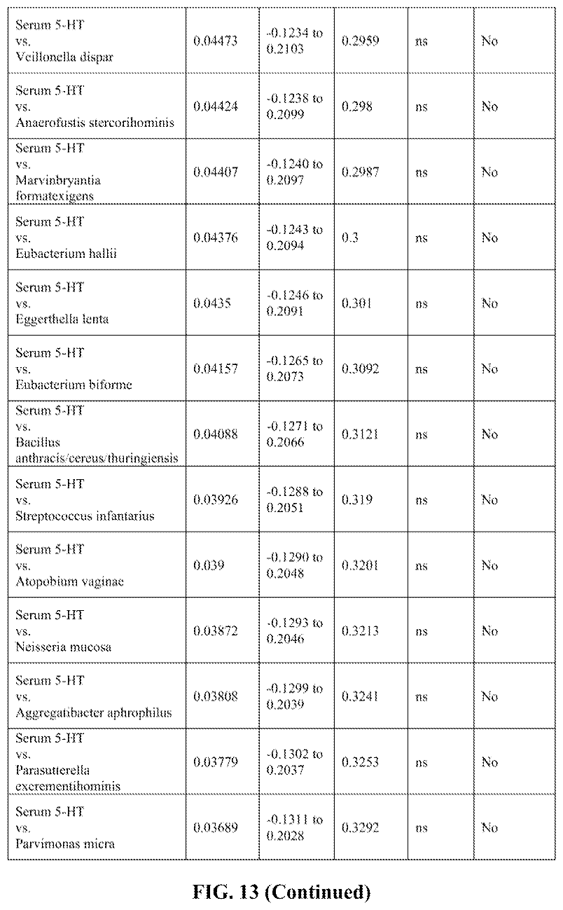

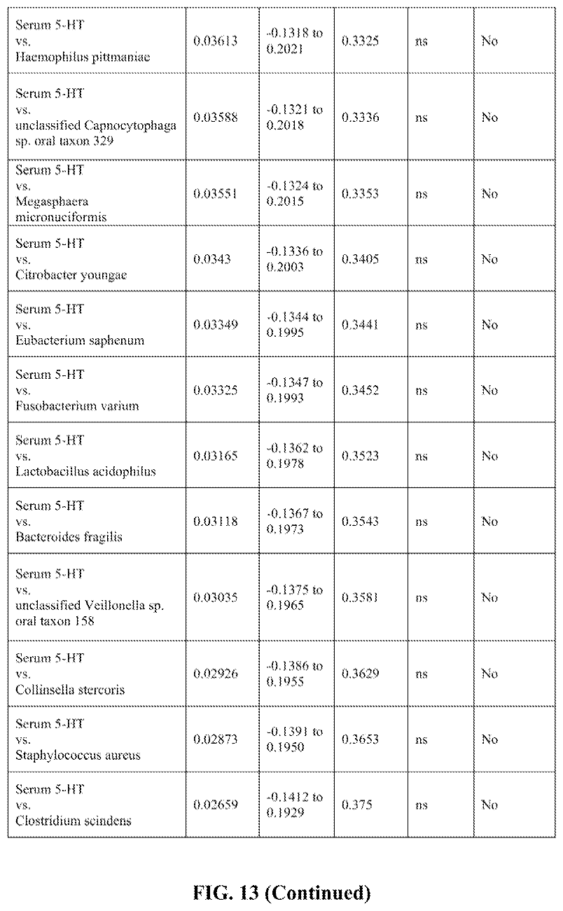

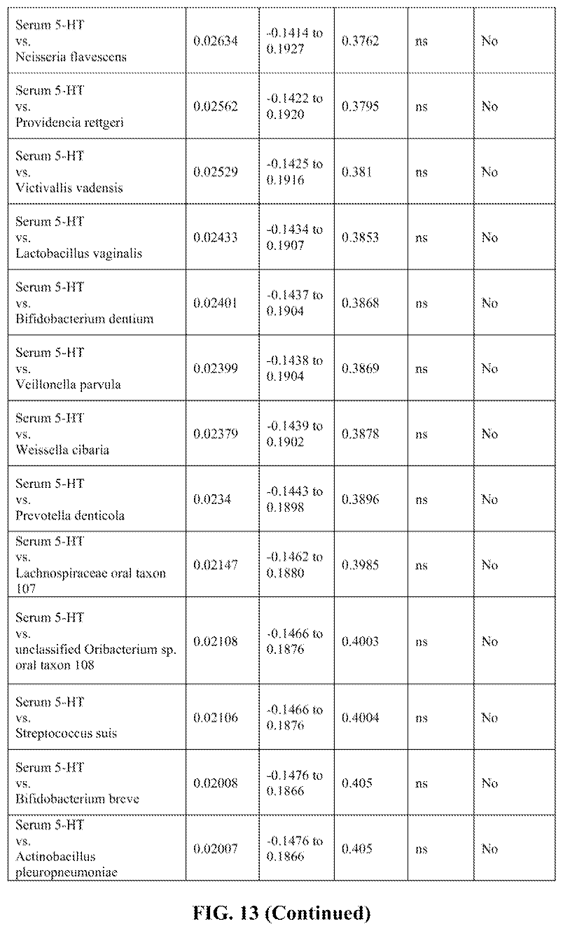

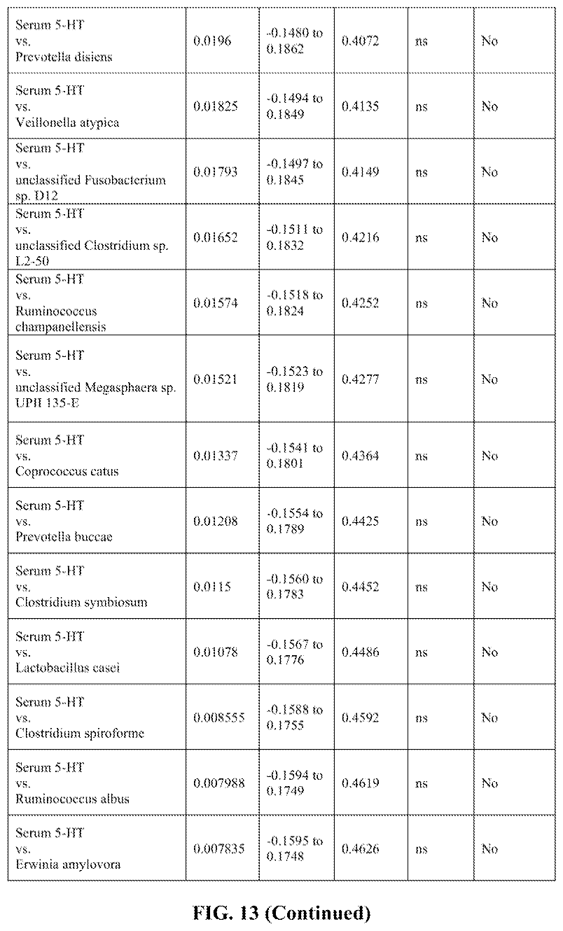

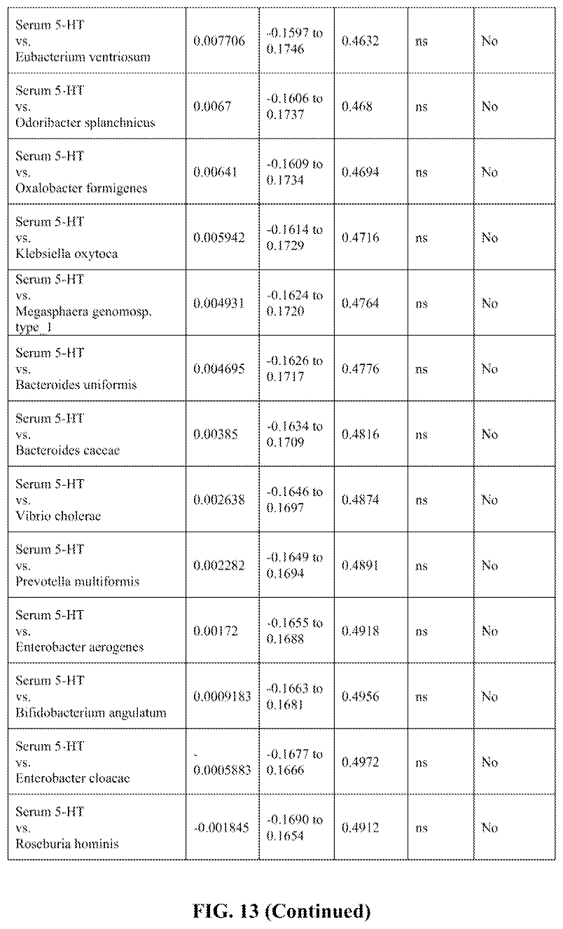

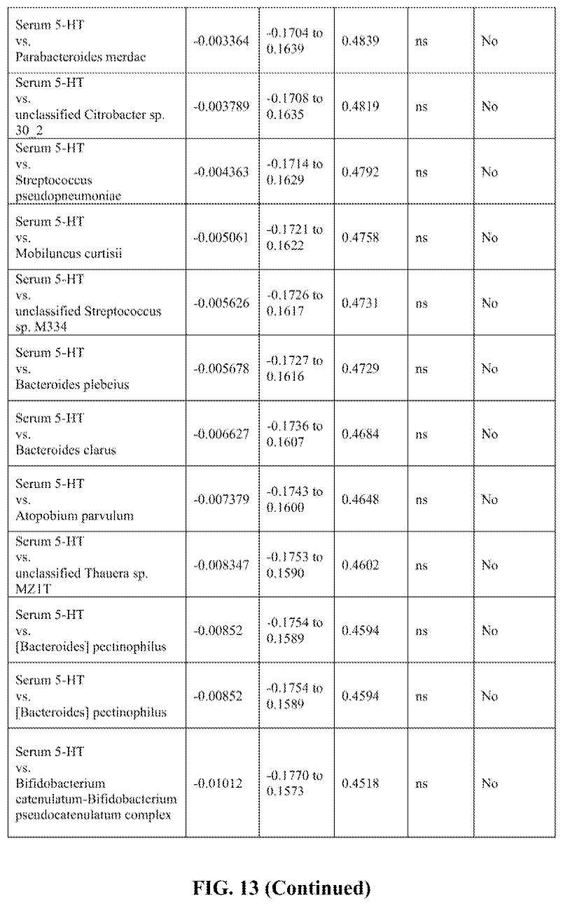

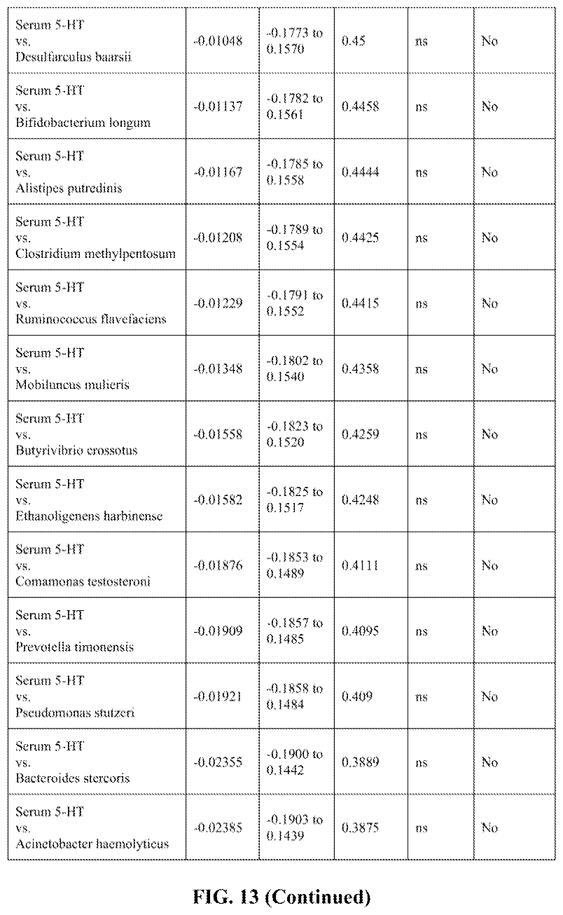

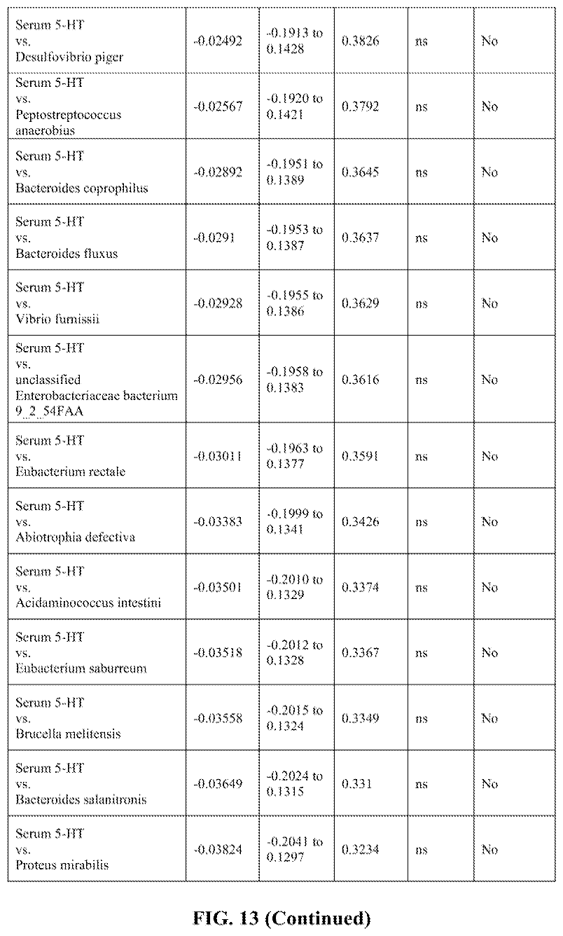

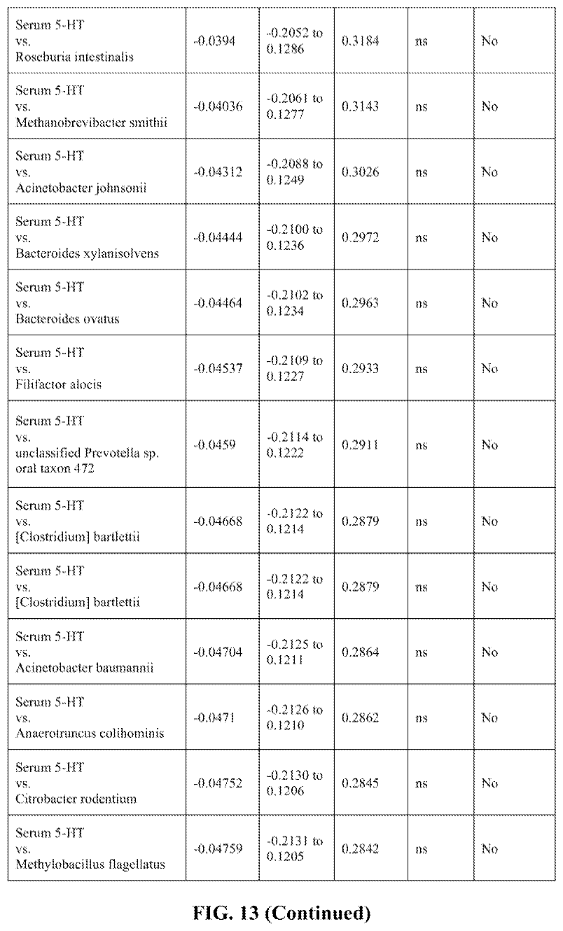

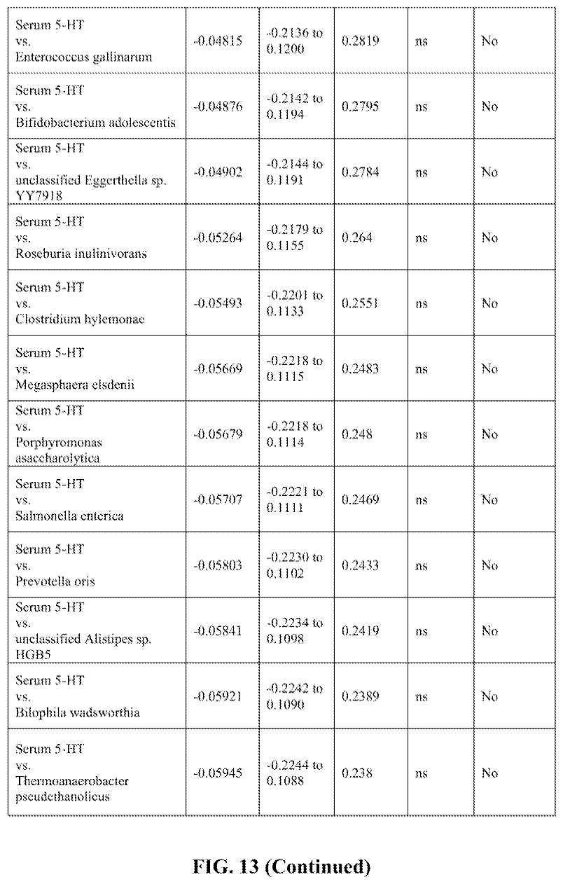

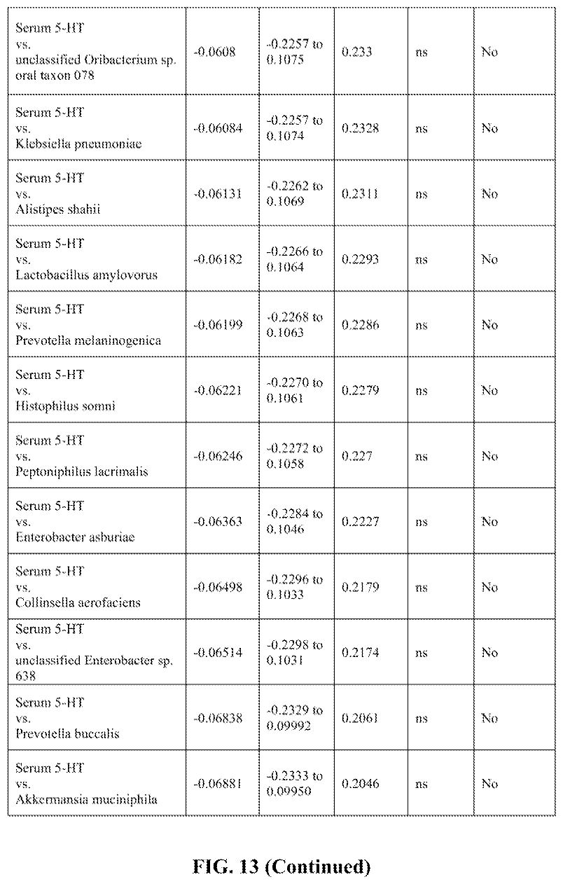

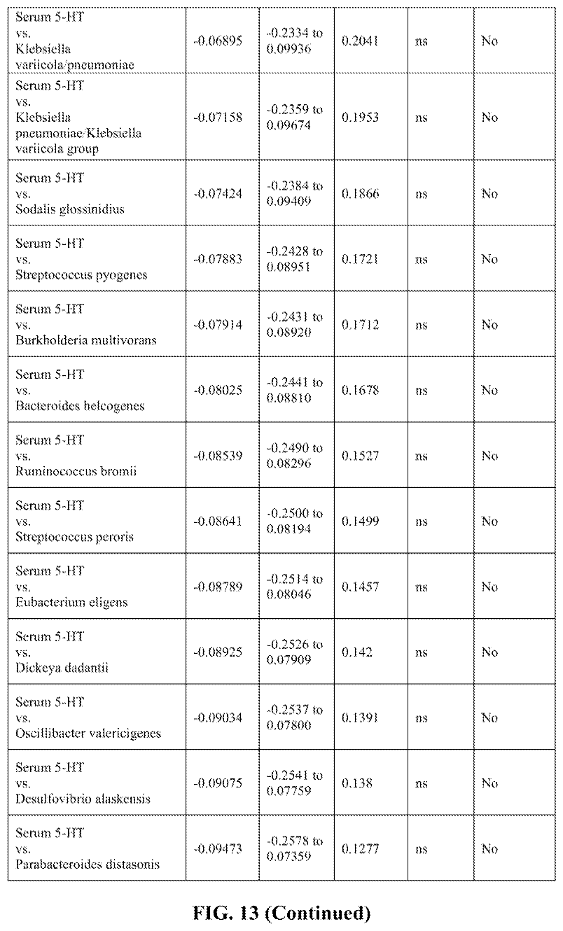

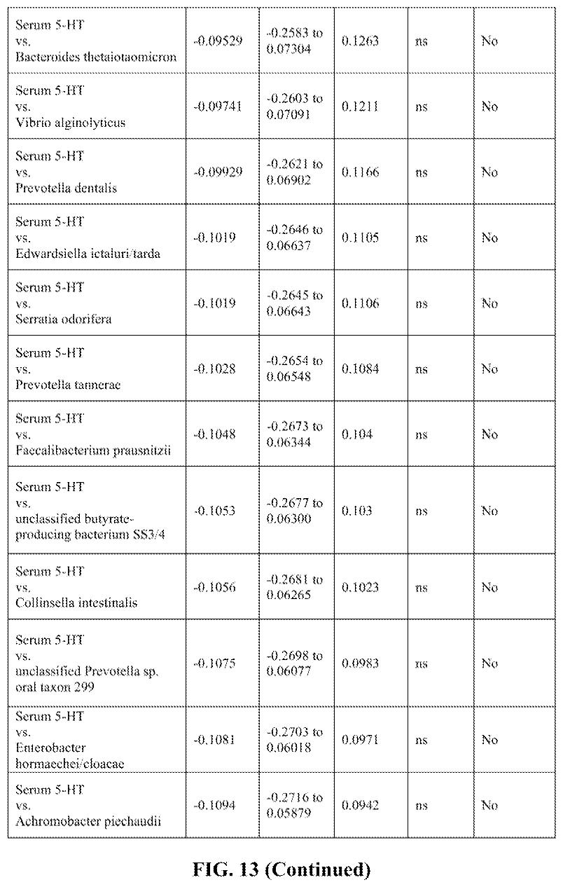

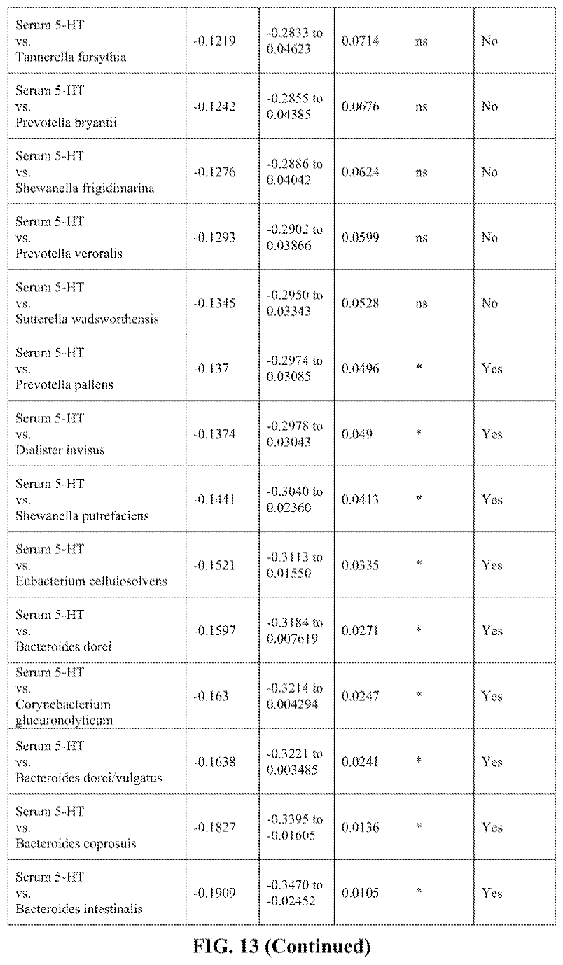

To identify the bacterial species responsible for 5-HT abnormalities in IBS-D, biological samples (serum, urine, and feces) obtained from patients with IBS-D and healthy controls (HC) were used. First, an increased level of peripheral 5-HT was found in the sera of patients with IBS-D (p=0.026, FIG. 1A). In line with the elevated serum 5-HT levels, there was also an increased level ofurine 5-hydroxyindole acetic acid (5-HIAA), a urinary biomarker of 5-HT (p=0.003, FIG. 1B). Second, for patients with IBS-D, correlation analyses were performed between the spectra of gut microbiota and serum 5-HT levels. It was found that a series of bacterial species that were positively correlated with serum 5-HT levels in patients with IBS-D (FIG. 1C and FIG. 13). Among these bacterial species, R. gnavus, a culturable gut bacterium with a relatively high abundance in the human gut microbiome, was significantly increased in patients with IBS-D (p<0.001, FIG. 1D). There was a positive correlation between the serum 5-HT levels and R. gnavus in this cohort (r=0.2109, p<0.01, FIG. 1E). Analyses of the clinical symptoms showed that R. gnavus abundance was positively correlated with the severity of IBS-D symptoms, as assessed using the Bristol stool scale (r=0.168, p=0.024, FIG. 1F). To further substantiate these findings, the observations were validated in another cohort of IBS-D and again showed that R. gnavus abundance was positively correlated with serum 5-HT levels and the frequency of diarrhea in IBS-D patients (r=0.281 and 0.271, p<0.01 both, FIG. 7A-B). Collectively, these data suggest that changes in the abundance of R. gnavus may be associated with altered 5-HT metabolism in patients with IBS-D.

The findings disclosed herein suggest that microbial metabolism of dietary aromatic amino acids by aromatic trace amine producers (e.g., R. gnavus) enhanced 5-HT biosynthesis to stimulate GI transit via a TAAR1-dependent mechanism, thereby contributing to diarrheal symptoms in patients with IBS-D. This study is in alignment with the previous studies showing that R. gnavus produces tryptamine by decarboxylating tryptophan and tryptamine induces ion secretion in colonic epithelial cells in vitro, suggesting that tryptamine derived from R. gnavus may play a role in GI motility.

Although gut microbial metabolites, including butyrate, propionate, tyramine, deoxycholate, and p-aminobenzoate, have been shown to stimulate 5-HT biosynthesis in vitro, the exact mechanism by which gut microbial metabolites affect 5-HT production and their roles in IBS development remain unclear. Recent studies have suggested that short fatty acids (butyrate and propionate) and deoxycholate do not change in the feces of IBS-D patients. This data suggests that these other metabolites are unlikely to be major contributors to the pathogenesis of IBS-D and highlight the importance of aromatic trace amines in the development of IBS-D in the patient cohort.

Elevated levels of tryptamine and its precursor Trp were previously reported in stool samples of non-Asian IBS-D patients, and the stimulatory effect of tryptamine on GI motility via the activation of epithelial 5-Hydroxytryptamine Receptor 4 (5-HTR4) has also been demonstrated. Interestingly, it was found that treatment of QGP-1 cells with the 5-HTR4 antagonist (Piboserod) did not restore 5-HT levels stimulated by phenethylamine or tryptamine to unstimulated levels 5-HTR4 antagonist. Though, there was modest reduction of tryptamine-stimulated 5-HT production (FIG. 11M). Therefore, aromatic trace amines may control GI motility by simultaneously regulating 5-HT production and its actions on 5-HT receptors, which may explain why the efficacy of current pharmacological approaches for treating IBS-D by targeting 5-HT receptors is not satisfactory, owing to the continuous activation of 5-HT biosynthesis by aromatic trace amines with a continuous supply of dietary amino acids. Therefore, developing new strategies to reduce the microbial transformation of dietary amino acids into aromatic trace amines may represent a feasible therapeutic approach for the treatment of IBS-D.

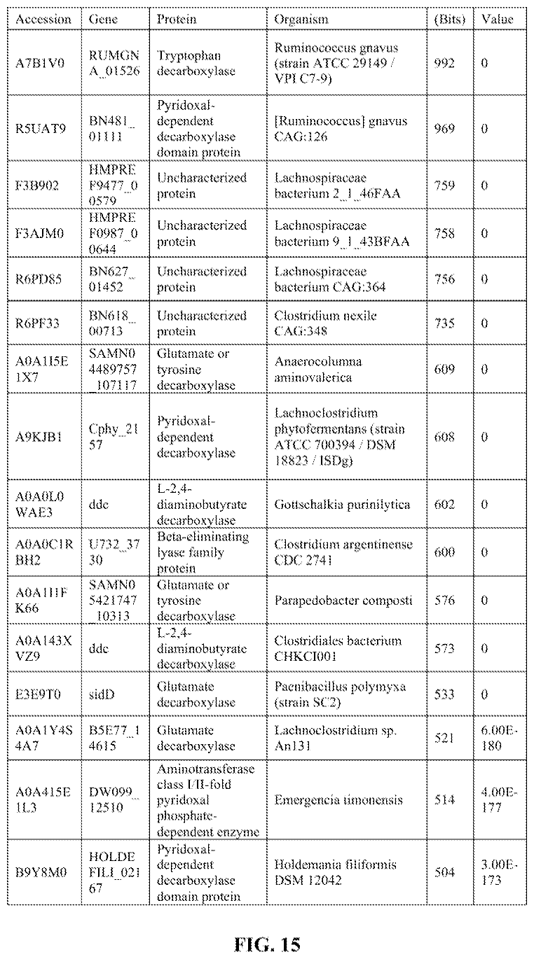

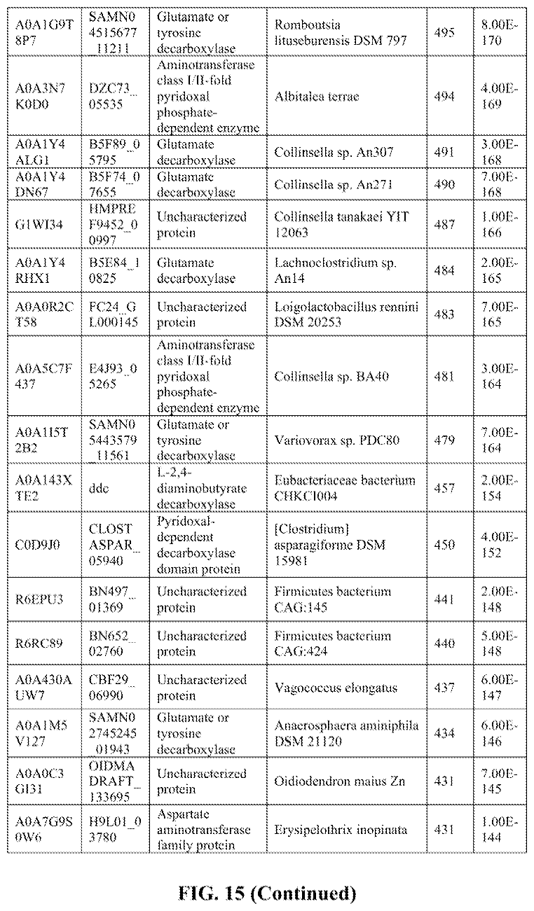

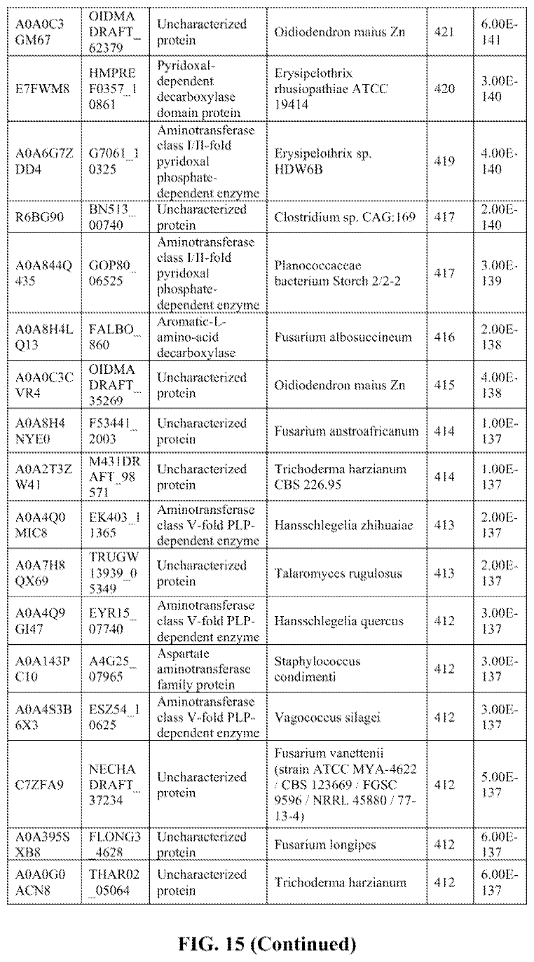

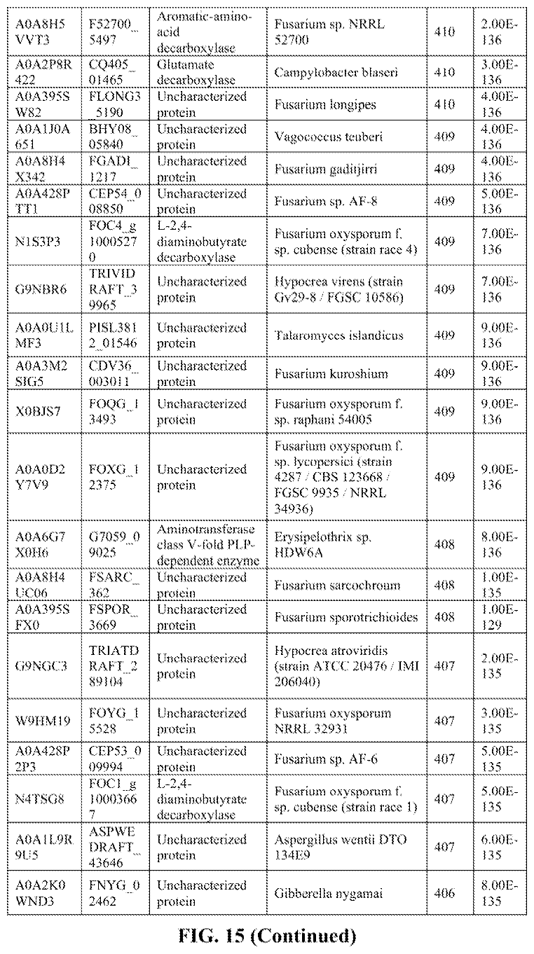

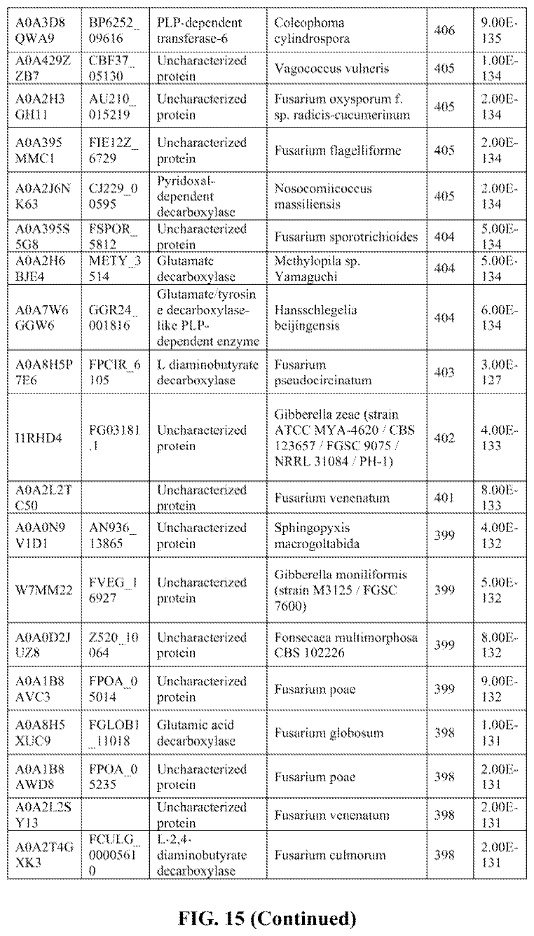

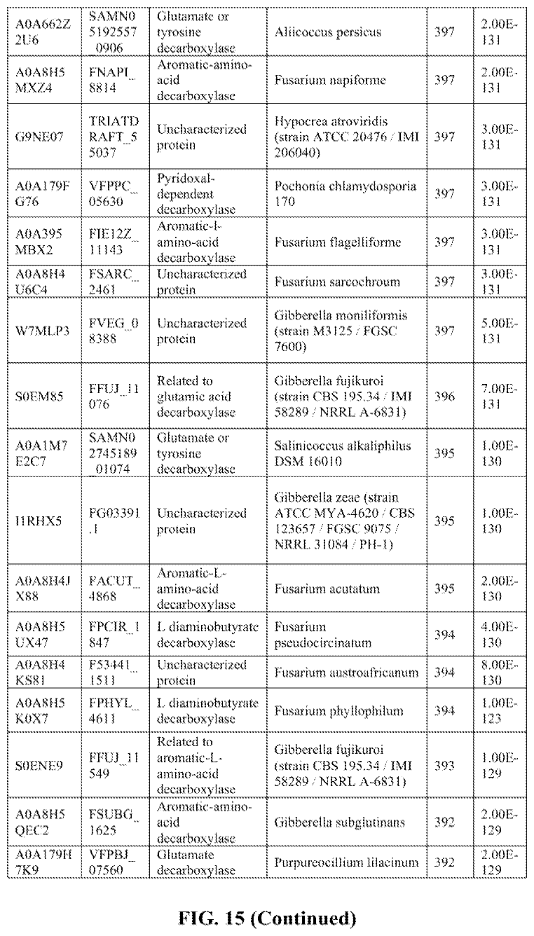

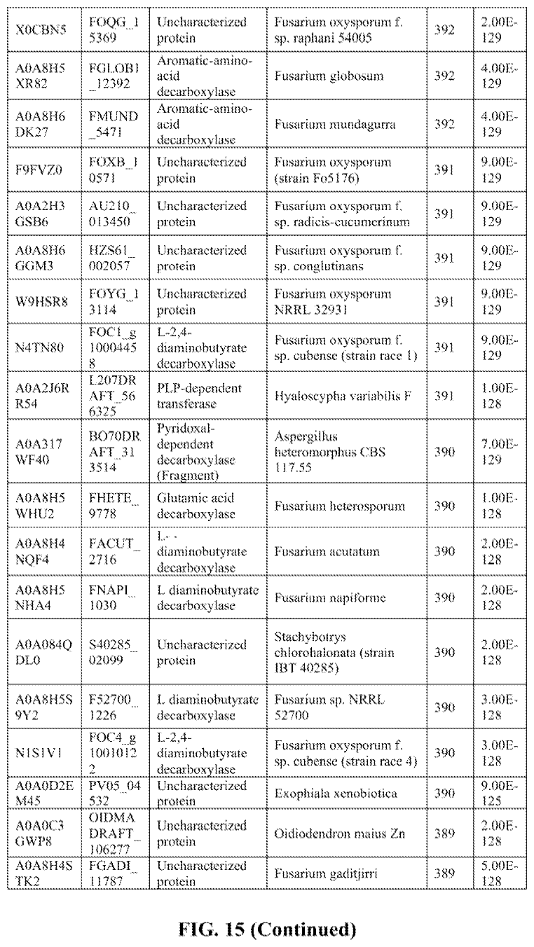

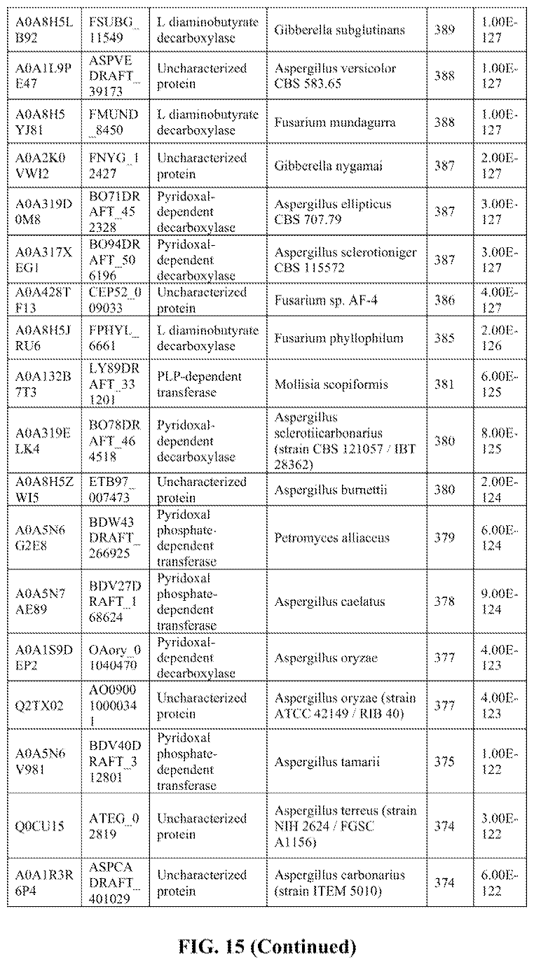

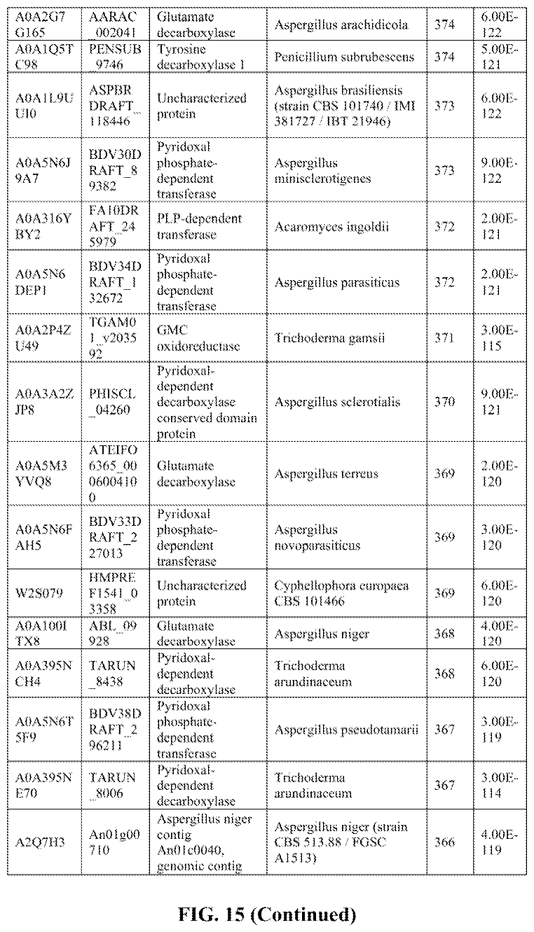

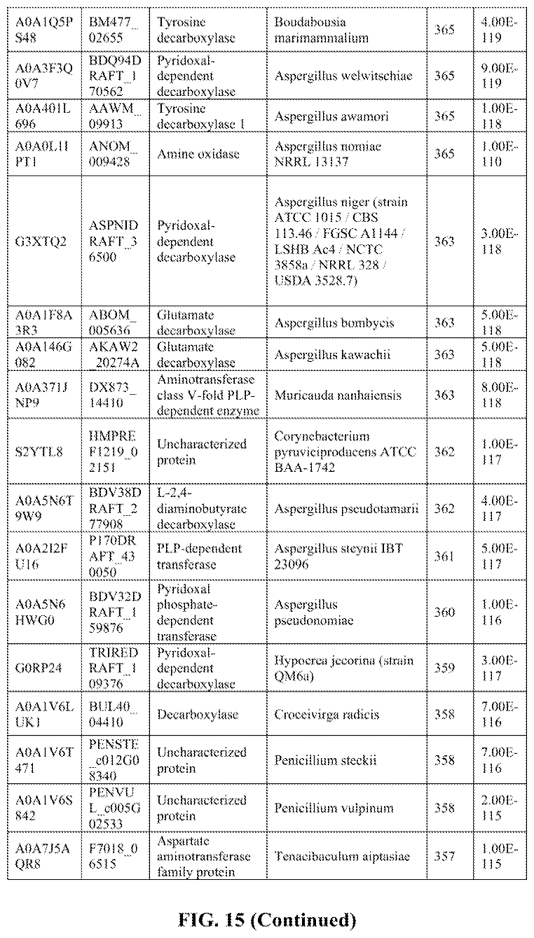

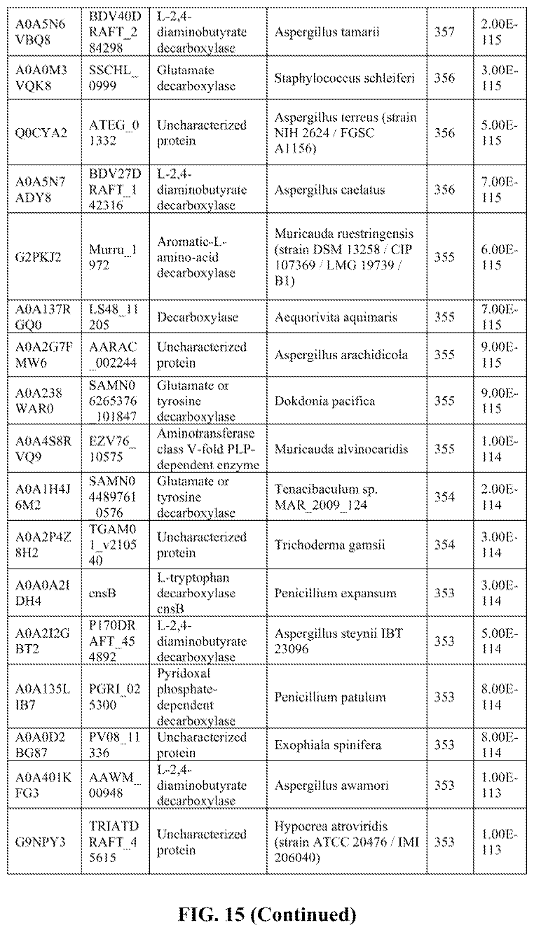

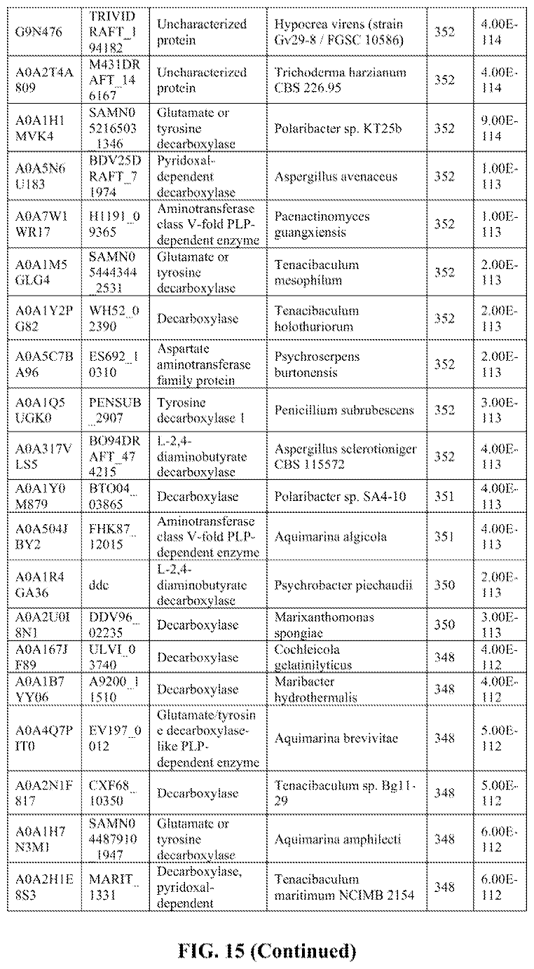

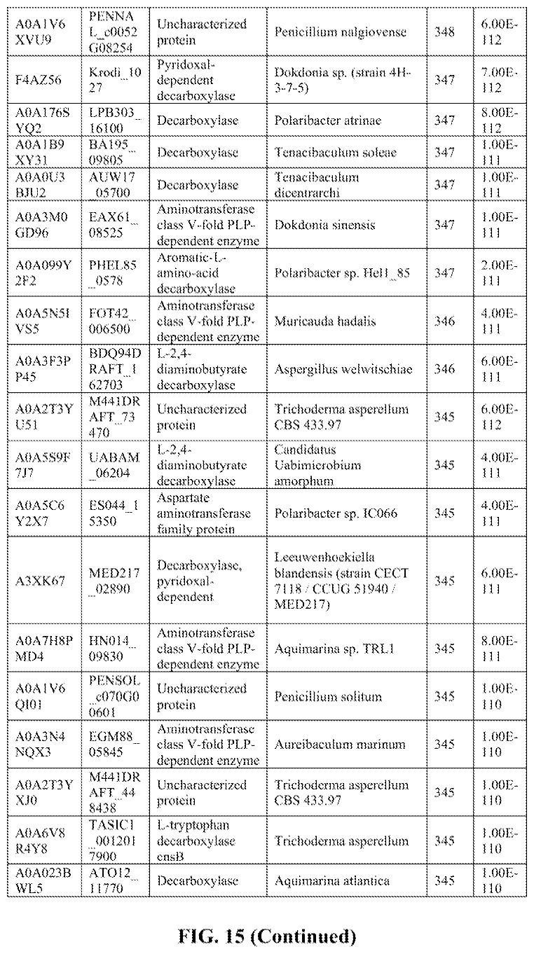

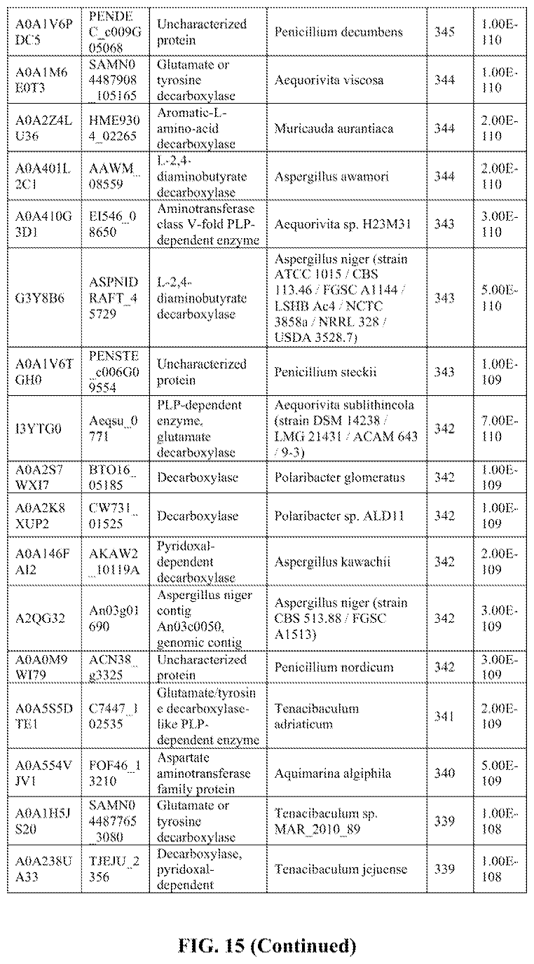

Interestingly, a recent study revealed that the elevation of intestinal 5-HT by oral supplementation or genetic approach enriched the abundance of spore-forming bacteria, especially for Ruminococcaceae, suggesting that aromatic trace amine producers (FIG. 15), such as R. gnavus ATCC 29149, can create a 5-HT-enriched gut microenvironment by stimulating 5-HT production, which in turn favors their competitive colonization in the GI tract, thus forming a positive feedback loop for 5-HT elevation in IBS-D patients. Therefore, an important therapeutic consideration is the inhibition of this feedback loop, which facilitates disease persistence.

R. gnavus has also been shown to be associated with inflammatory bowel disease (IBD) and exhibits pro-inflammatory properties by producing inflammatory polysaccharides, suggesting that R. gnavus may induce colitis by promoting inflammatory responses and impairing barrier functions. In contrast, our findings revealed that germ-free mice mono-associated with the R. gnavus (ATCC 29149) exhibited increased GI transit and colonic secretion without the presence of fecal occult blood, a major symptom of IBD, suggesting that R. gnavus may predominantly contribute to IBS-D symptoms. Indeed, our findings have demonstrated the positive association between R. gnavus and IBS-D in multiple cohorts with different ethnic groups. Additionally, this study identified a plausible mechanistic link between the gut microbial metabolites phenethylamine/tryptamine, and IBS-D pathogenesis. IBS-D is a heterogeneous disease and one subset is related to high phenethylamine/tryptamine levels in the gut. If so, fecal phenethylamine/tryptamine may be a potential biomarker for this particular subset of patients with IBS-D, facilitating the development of personalized treatment for IBS-D. This study further showed that antagonism of TAAR1 by specific antagonists alleviated IBS-D-like symptoms in antibiotic-treated mice transplanted with fecal microbiota from IBS-D patients with high phenethylamine/tryptamine levels. Unlike 5-HT inhibitors, which may cause severe side effects, neither genetic knockout of TAAR1 nor pharmacological inhibition of TAAR1 activity led to overt phenotypes in mice in this study. Therefore, TAAR1 may be a potential druggable target for the management of IBS-D by normalizing 5-HT dysfunction.

In this study, a TAAR1 antagonist was employed to determine the effects of R. gnavus ATCC 29149 on GI motility and 5-HT production. This investigation demonstrated that TAAR1 inhibition effectively abolished the stimulatory effect of phenethylamine/tryptamine on 5-HT biosynthesis in vitro and in vivo, suggesting that the in vivo action of phenethylamine/tryptamine is primarily mediated via TAAR1 in the context of gut motility. Genetically engineered R. gnavus without the TDC gene can be addressed in future studies to understand its other biological actions in the host.

What is claimed is:

1. A method of treating irritable bowel syndrome (IBS) in a subject in need thereof, the method comprising: administering a therapeutically effective amount of a trace amine-associated receptor 1 (TAAR1) inhibitor to the subject.

2. The method of claim 1, wherein the IBS is a diarrhea-predominant subtype (IBS-D).

3. The method of claim 1, wherein the TAAR1 inhibitor selectively binds TAAR1.

4. The method of claim 1, wherein the TAAR1 inhibitor is a small molecule.

5. The method of claim 1, wherein the TAAR1 inhibitor is a compound of Formula 1: 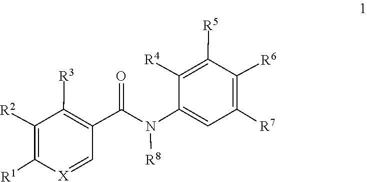
or a pharmaceutically acceptable salt thereof, wherein
n is 0, 1, 2 or 3,
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆alkyl, tetrahydro-furan-2-yl, morpholin-4-yl, pyrazol-1-yl, or —OC(O)—C₁-C₆ alkyl, or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R″ form together with the corresponding C-atoms a ring comprising —CH═CH—CH═CH—;
R₈ is hydrogen or C₁-C₆ alkyl,
X is —C(R⁹)═ or —N═;
R⁹ is hydrogen, C₁-C₆ alkoxy, NO₂ or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆alkyl, (CH₂)ₙ-phenyl optionally substituted by halogen or (CH₂)—O—C₁-C₆ alkyl.

6. The method of claim 1, wherein the TAAR1 inhibitor is N-(3-ethoxy-phenyl)-4-pyrrolidin-1-yl-3-trifluoromethyl-benzamide.

7. The method of claim 1, wherein the subject is a human, a non-human primate, a rodent, a canine, a feline, a bovine, or an equine.

8. The method of claim 1, wherein the subject is a human.

9. The method of claim 1, wherein a fecal sample obtained from the subject comprises a higher amount of one or more markers selected from the group consisting of Ruminococcus gnavus, phenethylamine, tryptamine and tyramine than an average amount of the one or more markers in fecal samples obtained from healthy controls.

10. The method of claim 1 further comprising the step of: providing a fecal sample obtained from the subject; determining the amount of one or more markers selected from the group consisting of phenethylamine, tryptamine and tyramine in the fecal sample; and determining based on the amount of the one or more markers in the fecal sample that the subject has IBS prior to the step of administering the TAAR1 inhibitor to the subject.

11. The method of claim 10, wherein the one or more markers is phenethylamine and tyramine.

12. The method of claim 1 further comprising the step of: providing a fecal sample obtained from the subject; determining the amount of Ruminococcus gnavus in the fecal sample; and determining based on the amount of Ruminococcus gnavus in the fecal sample that the subject has IBS prior to the step of administering the TAAR1 inhibitor to the subject.

13. A method of treating diarrhea-predominant irritable bowel syndrome (IBS-D) in a subject in need thereof, the method comprising: providing a fecal sample obtained from the subject; determining the amount of one or more markers selected from the group consisting of Ruminococcus gnavus, phenethylamine, tryptamine and tyramine in the fecal sample; determining based on the amount of the one or more markers in the fecal sample that the subject has IBS; and administering a therapeutically effective amount of a trace amine-associated receptor 1 (TAAR1) inhibitor to the subject.

14. The method of claim 13, wherein the TAAR1 inhibitor is a compound of Formula 1: 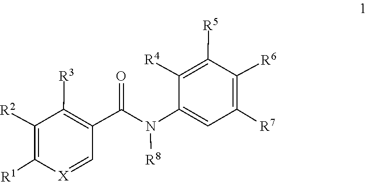
or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆alkyl, C₁-C₆alkoxy, C₁-C₆alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆alkoxy;
R¹ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂ or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

15. The method of claim 13, wherein the TAAR1 inhibitor is N-(3-ethoxy-phenyl)-4-pyrrolidin-1-yl-3-trifluoromethyl-benzamide.

16. A method for diagnosing irritable bowel syndrome (IBS) in a subject, the method comprising: providing a fecal sample obtained from the subject; determining the amount of one or more markers selected from the group consisting of Ruminococcus gnavus, phenethylamine, tryptamine and tyramine in the fecal sample; and determining based on the amount of the one or more markers in the fecal sample if the subject has IBS.

17. The method of claim 16, wherein the IBS is a diarrhea-predominant subtype (IBS-D).

18. The method of claim 16, wherein the one or more markers is phenethylamine and tyramine.

19. The method of claim 16 further comprising the step of administering a therapeutically effective amount of a trace amine-associated receptor 1 (TAAR1) inhibitor to the subject.

20. The method of claim 19, wherein the TAAR1 inhibitor is N-(3-ethoxy-phenyl)-4-pyrrolidin-1-yl-3-trifluoromethyl-benzamide.


SEQUENCE LISTING (5 sequences)

SEQ ID NO: 1 - DNA, 33 bp, Mus musculus
  source 1..33: mol_type = genomic DNA
ctgtacagtttaatggtgctcataattctgacc

SEQ ID NO: 2 - DNA, 31 bp, Mus musculus
  source 1..31: mol_type = genomic DNA
agcatagtagcggycaatggagatgaaagac

SEQ ID NO: 3 - DNA, 24 bp, Mus musculus
  source 1..24: mol_type = genomic DNA
gacaaaacgtagttggaagactga

SEQ ID NO: 4 - DNA, 25 bp, Mus musculus
  source 1..25: mol_type = genomic DNA
gtgtgcctagaaaccttaacatctg

SEQ ID NO: 5 - DNA, 24 bp, Mus musculus
  source 1..24: mol_type = genomic DNA
aatgtttgtgatagcgtggcaaag